SEQUENCE LISTING (169 sequences)

SEQ ID NO: 1 - protein, 321 aa, Homo sapiens
QERRFQLEGPESLTVQEGLCVLVPCRLPTTLPASYYGYGYWFLEGADVPVATNDPDEEVQEETRGRFHLLWDPRRKNCSLSIRDARRRDNAAYFFRLKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGV

SEQ ID NO: 2 - protein, 121 aa, Homo sapiens
QERRFQLEGPESLTVQEGLCVLVPCRLPTTLPASYYGYGYWFLEGADVPVATNDPDEEVQEETRGRFHLLWDPRRKNCSLSIRDARRRDNAAYFFRLKSKWMKYGYTSSKLSVRVMALTHR

SEQ ID NO: 3 - protein, 90 aa, Homo sapiens
PNISIPGTLESGHPSNLTCSVPWVCEQGTPPIFSWMSAAPTSLGPRTTQSSVLTITPRPQDHSTNLTCQVTFPGAGVTMERTIQLNVSYA

SEQ ID NO: 4 - protein, 110 aa, Homo sapiens
PQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGV

SEQ ID NO: 5 - protein, 10 aa, synthetic construct
GFSLTSYGVS

SEQ ID NO: 6 - protein, 16 aa, synthetic construct
VIWHDGSTSYHSTLIS

SEQ ID NO: 7 - protein, 9 aa, synthetic construct
DGYSGTFAY

SEQ ID NO: 8 - protein, 12 aa, synthetic construct
TASSSVSSSYLH

SEQ ID NO: 9 - protein, 7 aa, synthetic construct
STSILAS

SEQ ID NO: 10 - protein, 9 aa, synthetic construct
HQYHRSPYT

SEQ ID NO: 11 - protein, 10 aa, synthetic construct
GYIFTSYWMY

SEQ ID NO: 12 - protein, 17 aa, synthetic construct
NVNPYSGDTNYNEKFKT

SEQ ID NO: 13 - protein, 11 aa, synthetic construct
GDGLYYYAMDY

SEQ ID NO: 14 - protein, 11 aa, synthetic construct
KASQDINSYLT

SEQ ID NO: 15 - protein, 7 aa, synthetic construct
RASRLVD

SEQ ID NO: 16 - protein, 9 aa, synthetic construct
LQYDEFPLT

SEQ ID NO: 17 - protein, 10 aa, synthetic construct
GYTFTNYYIH

SEQ ID NO: 18 - protein, 17 aa, synthetic construct
MIHPISGSTNYNEKLKI

SEQ ID NO: 19 - protein, 12 aa, synthetic construct
RYYGSSYWYFDV

SEQ ID NO: 20 - protein, 10 aa, synthetic construct
RASSSVSYMN

SEQ ID NO: 21 - protein, 7 aa, synthetic construct
ATSNLAS

SEQ ID NO: 22 - protein, 9 aa, synthetic construct
QQWSSHPPT

SEQ ID NO: 23 - protein, 10 aa, synthetic construct
GYTFTTYGMS

SEQ ID NO: 24 - protein, 17 aa, synthetic construct
WINTYSGVPTYADDFKG

SEQ ID NO: 25 - protein, 10 aa, synthetic construct
LAYFGYYFDF

SEQ ID NO: 26 - protein, 11 aa, synthetic construct
KASQNVGSAVA

SEQ ID NO: 27 - protein, 7 aa, synthetic construct
SASNRYT

SEQ ID NO: 28 - protein, 9 aa, synthetic construct
QQYSSYPYT

SEQ ID NO: 29 - protein, 10 aa, synthetic construct
GFSFSTYVMS

SEQ ID NO: 30 - protein, 17 aa, synthetic construct
SISGGGSNTYYPDSVKG

SEQ ID NO: 31 - protein, 11 aa, synthetic construct
REVVVYWYFDV

SEQ ID NO: 32 - protein, 11 aa, synthetic construct
RASENIYSNLA

SEQ ID NO: 33 - protein, 7 aa, synthetic construct
SAKNLAD

SEQ ID NO: 34 - protein, 9 aa, synthetic construct
QHFWGTPLT

SEQ ID NO: 35 - protein, 10 aa, synthetic construct
GFSLTSYGVS

SEQ ID NO: 36 - protein, 16 aa, synthetic construct
IIWGDGSTNYHSALIS

SEQ ID NO: 37 - protein, 9 aa, synthetic construct
GLSDWYFDV

SEQ ID NO: 38 - protein, 10 aa, synthetic construct
RASSSVSYMH

SEQ ID NO: 39 - protein, 7 aa, synthetic construct
ATSNLAS

SEQ ID NO: 40 - protein, 9 aa, synthetic construct
QQWSSNPYT

SEQ ID NO: 41 - protein, 10 aa, synthetic construct
GYTFTTYGMS

SEQ ID NO: 42 - protein, 17 aa, synthetic construct
WINTYSGVPTYADDFKG

SEQ ID NO: 43 - protein, 10 aa, synthetic construct
LAYFGYYFDF

SEQ ID NO: 44 - protein, 11 aa, synthetic construct
KASQNVGSAVT

SEQ ID NO: 45 - protein, 7 aa, synthetic construct
SASNRYT

SEQ ID NO: 46 - protein, 9 aa, synthetic construct
QQYSSYPYT

SEQ ID NO: 47 - protein, 10 aa, synthetic construct
GFSLTSYGVH

SEQ ID NO: 48 - protein, 16 aa, synthetic construct
VIWGGGSTDYNAGFIS

SEQ ID NO: 49 - protein, 9 aa, synthetic construct
ALSDWYFDV

SEQ ID NO: 50 - protein, 10 aa, synthetic construct
RASSSVSYMH

SEQ ID NO: 51 - protein, 7 aa, synthetic construct
ATSNLAS

SEQ ID NO: 52 - protein, 9 aa, synthetic construct
QQWSSNPLT

SEQ ID NO: 53 - protein, 10 aa, synthetic construct
GYTFTDYYMN

SEQ ID NO: 54 - protein, 17 aa, synthetic construct
DINPKIGDTIYNQKFKG

SEQ ID NO: 55 - protein, 11 aa, synthetic construct
WDYYGSSLFDY

SEQ ID NO: 56 - protein, 11 aa, synthetic construct
KASQDVTTAVA

SEQ ID NO: 57 - protein, 7 aa, synthetic construct
WASTRHT

SEQ ID NO: 58 - protein, 9 aa, synthetic construct
QQHYSTPYT

SEQ ID NO: 59 - protein, 10 aa, synthetic construct
GYTLTTYGMS

SEQ ID NO: 60 - protein, 17 aa, synthetic construct
WINTYSGVPTYADEFKG

SEQ ID NO: 61 - protein, 10 aa, synthetic construct
LGYYGYYFDY

SEQ ID NO: 62 - protein, 11 aa, synthetic construct
RASQSIRNNLH

SEQ ID NO: 63 - protein, 7 aa, synthetic construct
YIFQSIS

SEQ ID NO: 64 - protein, 10 aa, synthetic construct
QQSDSWPHVT

SEQ ID NO: 65 - protein, 10 aa, synthetic construct
GFSLTSYGVN

SEQ ID NO: 66 - protein, 16 aa, synthetic construct
VIWGDGSTNYHSALIS

SEQ ID NO: 67 - protein, 9 aa, synthetic construct
ALSDWYFDV

SEQ ID NO: 68 - protein, 10 aa, synthetic construct
RASSSVSYMH

SEQ ID NO: 69 - protein, 7 aa, synthetic construct
ATSNLAS

SEQ ID NO: 70 - protein, 9 aa, synthetic construct
QQWSSNPYT

SEQ ID NO: 71 - protein, 10 aa, synthetic construct
GYTFTDYKMH

SEQ ID NO: 72 - protein, 17 aa, synthetic construct
NINPNNGGTSYNQKFKG

SEQ ID NO: 73 - protein, 12 aa, synthetic construct
RYYGSSSSYFDY

SEQ ID NO: 74 - protein, 10 aa, synthetic construct
SVSSSVSYMH

SEQ ID NO: 75 - protein, 7 aa, synthetic construct
DTSKLAS

SEQ ID NO: 76 - protein, 9 aa, synthetic construct
QQWSSNPPT

SEQ ID NO: 77 - protein, 10 aa, synthetic construct
GFTFSDYGIH

SEQ ID NO: 78 - protein, 17 aa, synthetic construct
YITSVSSTIYYADTVKG

SEQ ID NO: 79 - protein, 7 aa, synthetic construct
LDWYFDV

SEQ ID NO: 80 - protein, 10 aa, synthetic construct
SANSSVSFMH

SEQ ID NO: 81 - protein, 7 aa, synthetic construct
DTSKLAS

SEQ ID NO: 82 - protein, 9 aa, synthetic construct
QQWSSNPLT

SEQ ID NO: 83 - protein, 10 aa, synthetic construct
GYTFTSYWMH

SEQ ID NO: 84 - protein, 17 aa, synthetic construct
TIYPGNSDSTYNQKFKG

SEQ ID NO: 85 - protein, 13 aa, synthetic construct
EGTVVANYYGLDY

SEQ ID NO: 86 - protein, 10 aa, synthetic construct
RASSSVSYIH

SEQ ID NO: 87 - protein, 7 aa, synthetic construct
ATSNLAS

SEQ ID NO: 88 - protein, 9 aa, synthetic construct
QQWSSKPPT

SEQ ID NO: 89 - protein, 10 aa, synthetic construct
GFSLTSYSVH

SEQ ID NO: 90 - protein, 16 aa, synthetic construct
VIWSGGSTDYNAVFIS

SEQ ID NO: 91 - protein, 8 aa, synthetic construct
YSYYTMDY

SEQ ID NO: 92 - protein, 16 aa, synthetic construct
RSSQSLVLDNGNTYLH

SEQ ID NO: 93 - protein, 7 aa, synthetic construct
KVSNRFS

SEQ ID NO: 94 - protein, 9 aa, synthetic construct
SQSTHVPFT

SEQ ID NO: 95 - protein, 10 aa, synthetic construct
GYAFTSYWMH

SEQ ID NO: 96 - protein, 17 aa, synthetic construct
EIYPTNGGTTYNEKFKR

SEQ ID NO: 97 - protein, 8 aa, synthetic construct
EDFYAMDY

SEQ ID NO: 98 - protein, 16 aa, synthetic construct
KSSQSLLDSDGKTCLN

SEQ ID NO: 99 - protein, 7 aa, synthetic construct
LVSKLDS

SEQ ID NO: 100 - protein, 9 aa, synthetic construct
WQGTHFPYT

SEQ ID NO: 101 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 104 - protein, 107 aa, synthetic construct [sequence omitted]

SEQ ID NO: 105 - protein, 117 aa, synthetic construct
QVQLKESGPGLVAPSQSLSITCTVSGFSLTSYGVSWVRQPPGKGLEWLGIIWGDGSTNYHSALISRLSISKDKSKSQVFLKLNSLQADDTATYYCAKGLSDWYFDVWGTGTTVTVSS

SEQ ID NO: 106 - protein, 106 aa, synthetic construct
QIVLSQSPAILSASPGEKVTMTCRASSSVSYMHWYQQKPGSSPKPWIYATSNLASGVPARFSGSGSGTSYSLTISRVEAEDAATYYCQQWSSNPYTFGGGTKLEIK

SEQ ID NO: 107 - protein, 119 aa, synthetic construct
QIQLVQSGPELKKPGETVKISCKASGYTFTTYGMSWVKQAPGKGLKWMGWINTYSGVPTYADDFKGRFAFSLETSATTAYLQINNLKNEDTATYFCARLAYFGYYFDFWGQGTTLTVSS

SEQ ID NO: 108 - protein, 107 aa, synthetic construct
DIVMTQSQKFMSTTVGDRVSFTCKASQNVGSAVAWYQQKPGQSPKLLIYSASNRYTGVPDRFTGSGSGTDFTLTISNMQSEDLADYFCQQYSSYPYTFGGGTKLEIK

SEQ ID NO: 109 - protein, 119 aa, synthetic construct; identical to SEQ ID NO: 107

SEQ ID NO: 110 - protein, 107 aa, synthetic construct
DIVMTQSQKFMSTTVGDRVSFTCKASQNVGSAVTWYQQKPGQSPKLLIYSASNRYTGVPDRFTGSGSGTDFTLTISNMQSEDLADYFCQQYSSYPYTFGGGTKLEIK

SEQ ID NO: 111 - protein, 117 aa, synthetic construct
QVQLKQSGPGLVQPSQSLSITCTVSGFSLTSYGVHWVRQSPGKGLEWLGVIWGGGSTDYNAGFISRLSISKDNSKSQVFFKMNSLQADDTAIYYCARALSDWYFDVWGTGTTVTVSS

SEQ ID NO: 112 - protein, 106 aa, synthetic construct
QIVLSQSPAILSASPGEKVTMTCRASSSVSYMHWYQQKPGSSPKPWIYATSNLASGVPARFSGSGSGTSYSLTISRVEAEDAATYYCQQWSSNPLTFGAGTKLELK

SEQ ID NO: 113 - protein, 120 aa, synthetic construct
EVMLVESGGGLVKPGGSLKLSCAASGFSFSTYVMSWVRQTPEKRLEWVASISGGGSNTYYPDSVKGRFTISRDNAKNTLYLQMSSLRSEDTALYYCARREVVVYWYFDVWGTGTTVTVSS

SEQ ID NO: 114 - protein, 107 aa, synthetic construct
DIQMTQSPASLSVSVGETVTITCRASENIYSNLAWYQQKQGKSPQLLVFSAKNLADGVPSRFGGSGSGTQYSLKINSLQSEDFGTYYCQHFWGTPLTFGAGTKLELK

SEQ ID NO: 115 - protein, 120 aa, synthetic construct
EVQLQQSGPELMKPGASVKISCKASGYTFTDYYMNWVKQSHGKSLEWIGDINPKIGDTIYNQKFKGKATLTVDKSSSTAYMELRSLTSEDSAVYYCARWDYYGSSLFDYWGQGTTLTVSS

SEQ ID NO: 116 - protein, 107 aa, synthetic construct
DIVMTQSHKFMSTSVGDRVSITCKASQDVTTAVAWYQQKPGQSPKLLIYWASTRHTGVPDRFTGTGSGTDYTLTISNVQAEDLALYYCQQHYSTPYTFGGGTKLEIK

SEQ ID NO: 117 - protein, 119 aa, synthetic construct
QIQLVQSGPELKKPGETVKISCKASGYTLTTYGMSWVKQAPGKGLKWMGWINTYSGVPTYADEFKGRFAFSLETSASTAYLQINNLKNEDTATYFCARLGYYGYYFDYWGQGTTLTVSS

SEQ ID NO: 118 - protein, 108 aa, synthetic construct
DVVLTQSPATLSVTPGDSVSLSCRASQSIRNNLHWYQQKSHESPRLLIKYIFQSISGIPSRFSGSGSGTEFTLSINSVETEDFGMYFCQQSDSWPHVTFGAGTKLELI

SEQ ID NO: 119 - protein, 117 aa, synthetic construct
QVQLKESGPGLVAPSQSLSITCTVSGFSLTSYGVNWIRQPPGKGLEWLGVIWGDGSTNYHSALISRLSISKDNSKSQVFLKLNSLQTDDTATYYCAKALSDWYFDVWGTGTTVTVSS

SEQ ID NO: 120 - protein, 106 aa, synthetic construct; identical to SEQ ID NO: 106

SEQ ID NO: 121 - protein, 121 aa, synthetic construct
EVQLQQSGPELVKPGASVKMSCKASGYTFTDYKMHWVKQSHGKSLEWIGNINPNNGGTSYNQKFKGKATLTVNKSSSTAYMELRSLTSEDSAVYYCARRYYGSSSSYFDYWGQGTTLTVSS

SEQ ID NO: 122 - protein, 106 aa, synthetic construct
QIVLTQSPAIMSASPGEKVTMTCSVSSSVSYMHWYQQKSGTSPKRWIYDTSKLASGVPARFSGSGSGTSYSLTISSMEAEDAATYYCQQWSSNPPTFGAGTKLELK

SEQ ID NO: 123 - protein, 116 aa, synthetic construct
EVPLVESGGGLVKPGGSLKLSCAASGFTFSDYGIHWVRQAPEKGLEWVAYITSVSSTIYYADTVKGRFTISRDNAKNTLFLQMTSLRSEDTAMYYCAILDWYFDVWGTGTTVTVSS

SEQ ID NO: 124 - protein, 106 aa, synthetic construct
QIVLTQSPAIMSASPGEKVTMTCSANSSVSFMHWFQQKSGTSPKRWIYDTSKLASGVPARFSASGSGTSYSLTISSMEAEDAATYYCQQWSSNPLTFGAGTKVELK

SEQ ID NO: 125 - protein, 120 aa, synthetic construct
QVQLQQPGTELVKPGASVQLSCKASGYIFTSYWMYWVKQRPGQGLEWIGNVNPYSGDTNYNEKFKTKATLTVDKASSTAFIQLSGLTSEDSAVYFCARGDGLYYYAMDYWGQGTSVTVSS

SEQ ID NO: 126 - protein, 107 aa, synthetic construct
DIKMTQSPSSMYASLGERVTITCKASQDINSYLTWFQQKPGKSPKTLIYRASRLVDGVPSRFSGSGSGQDYSLTISSLEYEDMGIYYCLQYDEFPLTFGAGAKLELI

SEQ ID NO: 127 - protein, 117 aa, synthetic construct
QVQLKESGPGLVAPSQSLSITCTVSGFSLTSYGVSWVRKPPGKGLEWLGVIWHDGSTSYHSTLISRLNISRDNSKSQVFLKLNSLQTDDTATYYCASDGYSGTFAYWGQGTLVTVSA

SEQ ID NO: 128 - protein, 108 aa, synthetic construct
QIVVTQSPAIMSASLGERVTMTCTASSSVSSSYLHWYQQKPGSSPKLWIYSTSILASGVPARFSGSGSGTSYSLTVSSMEAEDAATYYCHQYHRSPYTFGGGSKLEIR

SEQ ID NO: 129 - protein, 122 aa, synthetic construct
EVQLQQSGTVLARPGASVKMSCKTSGYTFTSYWMHWVKQRPGQGLEWIGTIYPGNSDSTYNQKFKGKAKLTAVTSASTAYMEVSSLTNEDSAVYYCSREGTVVANYYGLDYWGQGTSVTVSS

SEQ ID NO: 130 - protein, 106 aa, synthetic construct
QIVLSQSPAILSASPGEKVTMTCRASSSVSYIHWYQQKPGSSPKPWIYATSNLASGVPARFSGSGSGTSYSLTISRVEAEDAATYYCQQWSSKPPTFGAGTKLELK

SEQ ID NO: 131 - protein, 121 aa, synthetic construct
QVQLQQPGAELVKPGASVKLSCKASGYTFTNYYIHWVKQRPGQGLEWIGMIHPISGSTNYNEKLKIKATLTVDKSSSTAYMQLSSLTSEDSAVYYCARRYYGSSYWYFDVWGTGTTVTVSS

SEQ ID NO: 132 - protein, 106 aa, synthetic construct
QIVLSQSPAILSASPGEKVTMTCRASSSVSYMNWYQQKPGSSPKPWIYATSNLASGVPARFSGSGSGTPYSLTISRVEAEDAATYYCQQWSSHPPTFGSGTKLEIK

SEQ ID NO: 133 - protein, 116 aa, synthetic construct
QVQLKQSGPGLVQPSQSLSITCTVSGFSLTSYSVHWVRQSPGKGLEWLGVIWSGGSTDYNAVFISRLSISRDNSKSQVFFKMNSLQADDSAIYYCASYSYYTMDYWGQGTSVTVSS

SEQ ID NO: 134 - protein, 112 aa, synthetic construct
DVVMTQTPLSLPVSLGDQASISCRSSQSLVLDNGNTYLHWYLQKPGQSPKLLIYKVSNRFSGVPDRFTGSGSGTDFTLKINRVEAEDLGVYFCSQSTHVPFTFGSGTKLEIK

SEQ ID NO: 135 - protein, 10 aa, synthetic construct
GFNIKDDYMH

SEQ ID NO: 136 - protein, 17 aa, synthetic construct
WIDLENGDTEYASKFQG

SEQ ID NO: 137 - protein, 12 aa, synthetic construct
PIGTVVAEGFAY

SEQ ID NO: 138 - protein, 11 aa, synthetic construct
RASENIYSNLA

SEQ ID NO: 139 - protein, 7 aa, synthetic construct
AATNLAD

SEQ ID NO: 140 - protein, 9 aa, synthetic construct
QHFWGTPYT

SEQ ID NO: 141 - protein, 10 aa, synthetic construct
GYTFTDYKMH

SEQ ID NO: 142 - protein, 17 aa, synthetic construct
NINPNNGGTSYNQKFKG

SEQ ID NO: 143 - protein, 12 aa, synthetic construct
RYYGSSSSYFDY

SEQ ID NO: 144 - protein, 11 aa, synthetic construct
RASQSIGTSIH

SEQ ID NO: 145 - protein, 7 aa, synthetic construct
YASESIS

SEQ ID NO: 146 - protein, 9 aa, synthetic construct
QQSNSWPTT

SEQ ID NO: 147 - protein, 10 aa, synthetic construct
GFTFSDYGMH

SEQ ID NO: 148 - protein, 17 aa, synthetic construct
YISSDSSTIYYADTVKG

SEQ ID NO: 149 - protein, 10 aa, synthetic construct
TIYYGNYFDY

SEQ ID NO: 150 - protein, 12 aa, synthetic construct
SASSSVSSSSLY

SEQ ID NO: 151 - protein, 7 aa, synthetic construct
SMSNLAS

SEQ ID NO: 152 - protein, 9 aa, synthetic construct
QQWSSNPLT

SEQ ID NO: 153 - protein, 121 aa, synthetic construct
EVQLQQSGAELVRPGASVKLSCSASGFNIKDDYMHWVKQRPGQGLEWIGWIDLENGDTEYASKFQGKATITADTSSNTAYLQLSSLTSEDTAVYYCTTPIGTVVAEGFAYWGQGTLVTVSA

SEQ ID NO: 154 - protein, 107 aa, synthetic construct
DIQMTQSPASLSVSVGETVTITCRASENIYSNLAWYQQKPGKSPQLLVYAATNLADGVPSRFSGSGSGTQYSLKINSLQSEDFGSYYCQHFWGTPYTFGGGTKLEIK

SEQ ID NO: 155 - protein, 121 aa, synthetic construct; identical to SEQ ID NO: 121

SEQ ID NO: 156 - protein, 107 aa, synthetic construct
DILLTQSPAILSVSPGERVSFSCRASQSIGTSIHWYQQRTNGSPRLLIKYASESISGIPSRFSGSGSGTDFTLSINSVESEDIADYYCQQSNSWPTTFGGGTKLEIK

SEQ ID NO: 157 - protein, 119 aa, synthetic construct
EELLVESGGGLVKPGGSLKLSCAASGFTFSDYGMHWVRQAPEKGLEWVAYISSDSSTIYYADTVKGRFTISRDNAKNTLFLQMTSLRSEDTAMYYCARTIYYGNYFDYWGQGTTLTVSS

SEQ ID NO: 158 - protein, 108 aa, synthetic construct
QIVLTQSPAIMSASPGERVTMTCSASSSVSSSSLYWYQQKSGSSPKLWIYSMSNLASGVPARFSGSGSGTSYSLTINSMEAEDAATYYCQQWSSNPLTFGAGTKLELK

SEQ ID NO: 159 - protein, 117 aa, synthetic construct
QVQLQESGPGLVKPSETLSLTCTVSGFSLTSYGVSWIRQPPGKGLEWIGVIWHDGSTSYHSTLISRVTISRDTSKNQVSLKLSSVTAADTAVYYCASDGYSGTFAYWGQGTLVTVSS

SEQ ID NO: 160 - protein, 108 aa, synthetic construct
QIVVTQSPATLSLSPGERATLSCTASSSVSSSYLHWYQQKPGQAPRLLIYSTSILASGIPARFSGSGSGTDFTLTISSLQPEDFAVYYCHQYHRSPYTFGQGTKLEIK

SEQ ID NO: 161 - protein, 117 aa, synthetic construct
QVQLQESGPGLVKPSETLSLTCTVSGFSLTSYGVSWIRQPPGKGLEWIGVIWHDGSTSYHPSLKSRVTISRDTSKNQVSLKLSSVTAADTAVYYCASDGYSGTFAYWGQGTLVTVSS

SEQ ID NO: 162 - protein, 108 aa, synthetic construct; identical to SEQ ID NO: 160

SEQ ID NO: 163 - protein, 117 aa, synthetic construct
QVQLQESGPGLVKPSETLSLTCTVSGFSLSSYGVSWIRQPPGKGLEWIGVIWHDGSTNYNPSLKSRVTISRDTSKNQVSLKLSSVTAADTAVYYCASDGYSGTFAYWGQGTLVTVSS

SEQ ID NO: 164 - protein, 108 aa, synthetic construct; identical to SEQ ID NO: 160

SEQ ID NO: 165 - protein, 446 aa, synthetic construct
QVQLQESGPGLVKPSETLSLTCTVSGFSLTSYGVSWIRQPPGKGLEWIGVIWHDGSTSYHPSLKSRVTISRDTSKNQVSLKLSSVTAADTAVYYCASDGYSGTFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 166 - protein, 447 aa, synthetic construct
QVQLQESGPGLVKPSETLSLTCTVSGFSLTSYGVSWIRQPPGKGLEWIGVIWHDGSTSYHPSLKSRVTISRDTSKNQVSLKLSSVTAADTAVYYCASDGYSGTFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 215 aa, synthetic construct
QIVVTQSPATLSLSPGERATLSCTASSSVSSSYLHWYQQKPGQAPRLLIYSTSILASGIPARFSGSGSGTDFTLTISSLQPEDFAVYYCHQYHRSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 7 aa, synthetic construct
GFSLTSY

SEQ ID NO: 169 - protein, 5 aa, synthetic construct
WHDGS

(19) United States
(12) Patent Application Publication
(10) Pub. No.: US 2024/0084005 A1
(43) Pub. Date: Mar. 14, 2024

(54) ANTI-SIGLEC-6 ANTIBODIES AND METHODS OF USE THEREOF

(71) Applicant: Allakos Inc., San Carlos, CA (US)

(72) Inventors: Bradford A. YOUNGBLOOD, Burlingame, CA (US); Julia SCHANIN, Emerald Hill, CA (US); John LEUNG, Redwood City, CA (US); Wouter KORVER, Mountain View, CA (US); Thuy LUU, San Carlos, CA (US); Emily C. BROCK, San Carlos, CA (US)

(21) Appl. No.: 18/346,013

(22) Filed: Jun. 30, 2023

Related U.S. Application Data
(63) Continuation-in-part of application No. PCT/US2022/076497, filed on Sep. 15, 2022.
(60) Provisional application No. 63/352,964, filed on Jun. 16, 2022.
(60) Provisional application No. 63/310,012, filed on Feb. 14, 2022.
(60) Provisional application No. 63/245,164, filed on Sep. 16, 2021.

(51) Int. Cl. C07K 16/28; A61P 37/06

(52) U.S. Cl. CPC C07K 16/2803; A61P 37/06; C07K 2317/24; C07K 2317/31; C07K 2317/56

(57) ABSTRACT

The present disclosure provides anti-Siglec-6 antibodies and their use in treating and preventing mast cell-mediated disorders, as well as compositions and kits comprising the anti-Siglec-6 antibodies.

### CROSS-REFERENCE TO RELATED APPLICATIONS

This application is a continuation-in-part of International Application No. PCT/US2022/076497, filed internationally on Sep. 15, 2022, which claims the benefit of U.S. Provisional Application Nos. 63/245,164, filed Sep. 16, 2021, 63/310,012, filed Feb. 14, 2022, and 63/352,964, filed Jun. 16, 2022, each of which is hereby incorporated by reference in its entirety.

### REFERENCE TO AN ELECTRONIC SEQUENCE LISTING

The contents of the electronic sequence listing (701712001520SEQLIST.xml; Size: 154,645 bytes; and Date of Creation: Jun. 29, 2023) are incorporated by reference herein in their entirety.

### FIELD OF THE INVENTION

This invention relates to anti-human Siglec-6 antibodies and methods of treating or preventing a disease mediated by cells expressing Siglec-6.

### BACKGROUND

Siglecs (sialic acid-binding immunoglobulin-like lectins) are single-pass transmembrane cell surface proteins found predominantly on leukocytes and that are characterized by their specificity for sialic acids attached to cell-surface glycoconjugates. The Siglec family contains at least 15 members that are found in mammals (Pillai et al., Annu Rev Immunol., 2012, 30:357-392). These members include sialoadhesion (Siglec-1), CD22 (Siglec-2), CD33 (Siglec-3), myelin associated glycoprotein (Siglec-4), Siglec-5, OBBP1 (Siglec-6), AIRM1 (Siglec-7), SAF-2 (Siglec-8), and CD329 (Siglec-9).

Siglec-6 (also known as CD327) is an inhibitory receptor that is selectively expressed on mast cells, e.g., human tissue mast cells, HMC-1 cells, and CD34+ derived human mast cells. Mast cells are considered pathogenic drivers of numerous autoimmune and inflammatory diseases, including but not limited to food allergy, mast cell activation syndrome, mastocytosis, IPF, COPD, and others. See, e.g., Yu, Y. et al. (2018) Front. Immunol. 9:2138; Yokoi, H. et al. (2006) Allergy 61:769-76; WO2005124358; and US PG Pub. Nos. US20060269556A1 and US20080267973A1. Engagement of Siglec-6 with antibody is thought to inhibit IgE-mediated mast cell activation.

There remains a need for antibodies that bind to human Siglec-6 with high affinity and show potent mast cell inhibitory activity. There is also a need for antibodies that bind to human Siglec-6 having different characteristics with regard to modulating Siglec-6 surface expression and mast cell activation/inhibition, which can affect their in vivo properties.

All references cited herein, including patent applications, patent publications, and scientific literature, are herein incorporated by reference in their entirety, as if each individual reference were specifically and individually indicated to be incorporated by reference.

### BRIEF SUMMARY

To meet this and other needs, the present disclosure provides, inter alia, antibodies that bind to human Siglec-6, as well as compositions, uses, and methods related thereto.

Accordingly, certain aspects of the present disclosure relate to humanized antibodies that bind to human Siglec-6, wherein the antibody binds to Domain 1 of an extracellular domain of human Siglec-6. In some embodiments, Domain 1 of the extracellular domain of human Siglec-6 comprises the amino acid sequence of SEQ ID NO:2.

Other aspects of the present disclosure relate to antibodies that bind to human Siglec-6, wherein the antibody binds to Domain 2 of an extracellular domain of human Siglec-6. In some embodiments, Domain 2 of the extracellular domain of human Siglec-6 comprises the amino acid sequence of SEQ ID NO:3. In some embodiments, the antibody is a humanized or human antibody.

Other aspects of the present disclosure relate to antibodies that bind to human Siglec-6, wherein the antibody binds to Domain 3 of an extracellular domain of human Siglec-6. In some embodiments, Domain 3 of the extracellular domain of human Siglec-6 comprises the amino acid sequence of SEQ ID NO:4. In some embodiments, the antibody is a humanized or human antibody.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:168, an HVR-H2 comprising the amino acid sequence of SEQ ID NO:169, and an HVR-H3 comprising the amino acid sequence of SEQ ID NO:7; and wherein the VL region comprises an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO:10. In some embodiments, the antibody is a humanized antibody.

Other aspects of the present disclosure relate to antibodies that bind to human Siglec-6, wherein the antibody comprises a heavy chain variable (VH) region and a light chain variable (VL) region; wherein the VH region comprises the amino acid sequence of SEQ ID NO:159; and the VL region comprises the amino acid sequence of SEQ ID NO:160.

Other aspects of the present disclosure relate to antibodies that bind to human Siglec-6, wherein the antibody comprises a heavy chain variable (VH) region and a light chain variable (VL) region; wherein the VH region comprises the amino acid sequence of SEQ ID NO:161; and the VL region comprises the amino acid sequence of SEQ ID NO:162.

Other aspects of the present disclosure relate to antibodies that bind to human Siglec-6, wherein the antibody comprises a heavy chain variable (VH) region and a light chain variable (VL) region; wherein the VH region comprises the amino acid sequence of SEQ ID NO:163; and the VL region comprises the amino acid sequence of SEQ ID NO:164.

Other aspects of the present disclosure relate to antibodies that bind to human Siglec-6, wherein the antibody comprises a heavy chain comprising the amino acid sequence of SEQ ID NO:165 or 166 and a light chain comprising the amino acid sequence of SEQ ID NO:167.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:89, an HVR-H2 comprising the amino acid sequence of SEQ ID NO:90, and an HVR-H3 comprising the amino acid sequence of SEQ ID NO:91; and wherein the VL region comprises an HVR-L1 comprising the amino acid sequence of SEQ ID NO:92, an HVR-L2 comprising the amino acid sequence of SEQ ID NO:93, and an HVR-L3 comprising the amino acid sequence of SEQ ID NO:94.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:5, an HVR-H2 comprising the amino acid sequence of SEQ ID NO:6, and an HVR-H3 comprising the amino acid sequence of SEQ ID NO:7; and wherein the VL region comprises an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO:10.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:83, an HVR-H2 comprising the amino acid sequence of SEQ ID NO:84, and an HVR-H3 comprising the amino acid sequence of SEQ ID NO:85; and wherein the VL region comprises an HVR-L1 comprising the amino acid sequence of SEQ ID NO:86, an HVR-L2 comprising the amino acid sequence of SEQ ID NO:87, and an HVR-L3 comprising the amino acid sequence of SEQ ID NO:88.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:11, an HVR-H2 comprising the amino acid sequence of SEQ ID NO:12, and an HVR-H3 comprising the amino acid sequence of SEQ ID NO:13; and wherein the VL region comprises an HVR-L1 comprising the amino acid sequence of SEQ ID NO:14, an HVR-L2 comprising the amino acid sequence of SEQ ID NO:15, and an HVR-L3 comprising the amino acid sequence of SEQ ID NO:16.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:77, an HVR-H2 comprising the amino acid sequence of SEQ ID NO:78, and an HVR-H3 comprising the amino acid sequence of SEQ ID NO:79; and wherein the VL region comprises an HVR-L1 comprising the amino acid sequence of SEQ ID NO:80, an HVR-L2 comprising the amino acid sequence of SEQ ID NO:81, and an HVR-L3 comprising the amino acid sequence of SEQ ID NO:82.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:17, an HVR-H2 comprising the amino acid sequence of SEQ ID NO:18, and an HVR-H3 comprising the amino acid sequence of SEQ ID NO:19; and wherein the VL region comprises an HVR-L1 comprising the amino acid sequence of SEQ ID NO:20, an HVR-L2 comprising the amino acid sequence of SEQ ID NO:21, and an HVR-L3 comprising the amino acid sequence of SEQ ID NO:22.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:71, an HVR-H2 comprising the amino acid sequence of SEQ ID NO:72, and an HVR-H3 comprising the amino acid sequence of SEQ ID NO:73; and wherein the VL region comprises an HVR-L1 comprising the amino acid sequence of SEQ ID NO:74, an HVR-L2 comprising the amino acid sequence of SEQ ID NO:75, and an HVR-L3 comprising the amino acid sequence of SEQ ID NO:76.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:53, an HVR-H2 comprising the amino acid sequence of SEQ ID NO:54, and an HVR-H3 comprising the amino acid sequence of SEQ ID NO:55; and wherein the VL region comprises an HVR-L1 comprising the amino acid sequence of SEQ ID NO:56, an HVR-L2 comprising the amino acid sequence of SEQ ID NO:57, and an HVR-L3 comprising the amino acid sequence of SEQ ID NO:58.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:29, an HVR-H2 comprising the amino acid sequence of SEQ ID NO:30, and an HVR-H3 comprising the amino acid sequence of SEQ ID NO:31; and wherein the VL region comprises an HVR-L1 comprising the amino acid sequence of SEQ ID NO:32, an HVR-L2 comprising the amino acid sequence of SEQ ID NO:33, and an HVR-L3 comprising the amino acid sequence of SEQ ID NO:34.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:23, an HVR-H2 comprising the amino acid sequence of SEQ ID NO:24, and an HVR-H3 comprising the amino acid sequence of SEQ ID NO:25; and wherein the VL region comprises an HVR-L1 comprising the amino acid sequence of SEQ ID NO:26, an HVR-L2 comprising the amino acid sequence of SEQ ID NO:27, and an HVR-L3 comprising the amino acid sequence of SEQ ID NO:28.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:35, an HVR-H2 comprising the amino acid sequence of SEQ ID NO:36, and an HVR-H3 comprising the amino acid sequence of SEQ ID NO:37; and wherein the VL region comprises an HVR-L1 comprising the amino acid sequence of SEQ ID NO:38, an HVR-L2 comprising the amino acid sequence of SEQ ID NO:39, and an HVR-L3 comprising the amino acid sequence of SEQ ID NO:40.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:41, an HVR-H2 comprising the amino acid sequence of SEQ ID NO:42, and an HVR-H3 comprising the amino acid sequence of SEQ ID NO:43; and wherein the VL region comprises an HVR-L1 comprising the amino acid sequence of SEQ ID NO:44, an HVR-L2 comprising the amino acid sequence of SEQ ID NO:45, and an HVR-L3 comprising the amino acid sequence of SEQ ID NO:46.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:47, an HVR-H2 comprising the amino acid sequence of SEQ ID NO:48, and an HVR-H3 comprising the amino acid sequence of SEQ ID NO:49; and wherein the VL region comprises an HVR-L1 comprising the amino acid sequence of SEQ ID NO:50, an HVR-L2 comprising the amino acid sequence of SEQ ID NO:51, and an HVR-L3 comprising the amino acid sequence of SEQ ID NO:52.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:59, an HVR-H2 comprising the amino acid sequence of SEQ ID NO:60, and an HVR-H3 comprising the amino acid sequence of SEQ ID NO:61; and wherein the VL region comprises an HVR-L1 comprising the amino acid sequence of SEQ ID NO:62, an HVR-L2 comprising the amino acid sequence of SEQ ID NO:63, and an HVR-L3 comprising the amino acid sequence of SEQ ID NO:64.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:65, an HVR-H2 comprising the amino acid sequence of SEQ ID NO:66, and an HVR-H3 comprising the amino acid sequence of SEQ ID NO:67; and wherein the VL region comprises an HVR-L1 comprising the amino acid sequence of SEQ ID NO:68, an HVR-L2 comprising the amino acid sequence of SEQ ID NO:69, and an HVR-L3 comprising the amino acid sequence of SEQ ID NO:70.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:135, an HVR-H2 comprising the amino acid sequence of SEQ ID NO:136, and an HVR-H3 comprising the amino acid sequence of SEQ ID NO:137; and wherein the VL region comprises an HVR-L1 comprising the amino acid sequence of SEQ ID NO:138, an HVR-L2 comprising the amino acid sequence of SEQ ID NO:139, and an HVR-L3 comprising the amino acid sequence of SEQ ID NO:140.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:141, an HVR-H2 comprising the amino acid sequence of SEQ ID NO:142, and an HVR-H3 comprising the amino acid sequence of SEQ ID NO:143; and wherein the VL region comprises an HVR-L1 comprising the amino acid sequence of SEQ ID NO:144, an HVR-L2 comprising the amino acid sequence of SEQ ID NO:145, and an HVR-L3 comprising the amino acid sequence of SEQ ID NO:146.

Other aspects of the present disclosure relate to antibodies that bind to human Siglec-6, wherein the antibody comprises a heavy chain variable (VH) region and a light chain variable (VL) region; wherein the VH region comprises an HVR-H1 comprising the amino acid sequence of SEQ ID NO:147, an HVR-H2 comprising the amino acid sequence of SEQ ID NO:148, and an HVR-H3 comprising the amino acid sequence of SEQ ID NO:149; and wherein the VL region comprises an HVR-L1 comprising the amino acid sequence of SEQ ID NO:150, an HVR-L2 comprising the amino acid sequence of SEQ ID NO:151, and an HVR-L3 comprising the amino acid sequence of SEQ ID NO:152.

In some embodiments according to any of the embodiments described herein, the antibody binds to the extracellular domain of human Siglec-6 when expressed on a surface of a human mast cell. In some embodiments, binding of the antibody to the extracellular domain of human Siglec-6 when expressed on a surface of a human mast cell leads to a reduced level of Siglec-6 on the cell surface. In some embodiments, the antibody comprises an Fc region, and binding of the antibody to the extracellular domain of human Siglec-6 when expressed on a surface of a human mast cell leads to a reduced level of Siglec-6 on the human mast cell surface in the presence a cell expressing an Fc receptor. In some embodiments, binding of the antibody to the extracellular domain of human Siglec-6 when expressed on a surface of a human mast cell induces dimerization and internalization of Siglec-6. In some embodiments, binding of the antibody to the extracellular domain of human Siglec-6 when expressed on a surface of a human mast cell does not lead to a reduced level of Siglec-6 on the cell surface. In some embodiments, binding of the antibody to the extracellular domain of human Siglec-6 inhibits activation of the mast cell. In some embodiments, binding of the antibody to the extracellular domain of human Siglec-6 inhibits expression of CD63 by the mast cell. In some embodiments, binding of the antibody to the extracellular domain of human Siglec-6 inhibits release of IL-13 by the mast cell. In some embodiments, binding of the antibody to the extracellular domain of human Siglec-6 inhibits release of IL-8 by the mast cell. In some embodiments, binding of the antibody to the extracellular domain of human Siglec-6 inhibits release of CCL4 by the mast cell. In some embodiments, binding of the antibody to the extracellular domain of human Siglec-6 inhibits release of CCL2 by the mast cell. In some embodiments, binding of the antibody to the extracellular domain of human Siglec-6 inhibits release of active tryptase by the mast cell. In some embodiments, binding of the antibody to the extracellular domain of human Siglec-6 inhibits release of IL-6 by the mast cell. In some embodiments, binding of the antibody to the extracellular domain of human Siglec-6 inhibits release of histamine by the mast cell. In some embodiments, binding of the antibody to the extracellular domain of human Siglec-6 inhibits release of chymase by the mast cell. In some embodiments, binding of the antibody to the extracellular domain of human Siglec-6 depletes mast cells expressing human Siglec-6 in vivo or in vitro. In some embodiments, binding of the antibody to the extracellular domain of human Siglec-6 induces antibody-dependent cell-mediated cytotoxicity (ADCC) and/or antibody-dependent cell-mediated phagocytosis (ADCP) targeting the cell that expresses the extracellular domain of human Siglec-6 (e.g., on its cell surface) in vivo or in vitro, e.g., in the presence of effector cells.

In some embodiments according to any of the embodiments described herein, the antibody binds to the extracellular domain of human Siglec-6 with an equilibrium dissociation constant (K_{D}) of about 250 pM or less, about 100 pM or less, about 10 pM or less, or about 1 pM. In some embodiments, the antibody comprises a heavy chain variable (VH) region and a light chain variable (VL) region; wherein the VH region comprises an HVR-H1 comprising the amino acid sequence of SEQ ID NO:89, an HVR-H2 comprising the amino acid sequence of SEQ ID NO:90, and an HVR-H3 comprising the amino acid sequence of SEQ ID NO:91; and wherein the VL region comprises an HVR-L1 comprising the amino acid sequence of SEQ ID NO:92, an HVR-L2 comprising the amino acid sequence of SEQ ID NO:93, and an HVR-L3 comprising the amino acid sequence of SEQ ID NO:94. In some embodiments, the antibody comprises a heavy chain variable (VH) region and a light chain variable (VL) region; wherein the VH region comprises an HVR-H1 comprising the amino acid sequence of SEQ ID NO:5, an HVR-H2 comprising the amino acid sequence of SEQ ID NO:6, and an HVR-H3 comprising the amino acid sequence of SEQ ID NO:7; and wherein the VL region comprises an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO:10. In some embodiments, the antibody comprises a heavy chain variable (VH) region and a light chain variable (VL) region; wherein the VH region comprises an HVR-H1 comprising the amino acid sequence of SEQ ID NO:83, an HVR-H2 comprising the amino acid sequence of SEQ ID NO:84, and an HVR-H3 comprising the amino acid sequence of SEQ ID NO:85; and wherein the VL region comprises an HVR-L1 comprising the amino acid sequence of SEQ ID NO:86, an HVR-L2 comprising the amino acid sequence of SEQ ID NO:87, and an HVR-L3 comprising the amino acid sequence of SEQ ID NO:88. In some embodiments, the antibody comprises a heavy chain variable (VH) region and a light chain variable (VL) region; wherein the VH region comprises an HVR-H1 comprising the amino acid sequence of SEQ ID NO:11, an HVR-H2 comprising the amino acid sequence of SEQ ID NO:12, and an HVR-H3 comprising the amino acid sequence of SEQ ID NO:13; and wherein the VL region comprises an HVR-L1 comprising the amino acid sequence of SEQ ID NO:14, an HVR-L2 comprising the amino acid sequence of SEQ ID NO:15, and an HVR-L3 comprising the amino acid sequence of SEQ ID NO:16. In some embodiments, the antibody comprises a heavy chain variable (VH) region and a light chain variable (VL) region; wherein the VH region comprises an HVR-H1 comprising the amino acid sequence of SEQ ID NO:77, an HVR-H2 comprising the amino acid sequence of SEQ ID NO:78, and an HVR-H3 comprising the amino acid sequence of SEQ ID NO:79; and wherein the VL region comprises an HVR-L1 comprising the amino acid sequence of SEQ ID NO:80, an HVR-L2 comprising the amino acid sequence of SEQ ID NO:81, and an HVR-L3 comprising the amino acid sequence of SEQ ID NO:82. In some embodiments, the antibody comprises a heavy chain variable (VH) region and a light chain variable (VL) region; wherein the VH region comprises an HVR-H1 comprising the amino acid sequence of SEQ ID NO:17, an HVR-H2 comprising the amino acid sequence of SEQ ID NO:18, and an HVR-H3 comprising the amino acid sequence of SEQ ID NO:19; and wherein the VL region comprises an HVR-L1 comprising the amino acid sequence of SEQ ID NO:20, an HVR-L2 comprising the amino acid sequence of SEQ ID NO:21, and an HVR-L3 comprising the amino acid sequence of SEQ ID NO:22. In some embodiments, the antibody comprises a heavy chain variable (VH) region and a light chain variable (VL) region; wherein the VH region comprises an HVR-H1 comprising the amino acid sequence of SEQ ID NO:71, an HVR-H2 comprising the amino acid sequence of SEQ ID NO:72, and an HVR-H3 comprising the amino acid sequence of SEQ ID NO:73; and wherein the VL region comprises an HVR-L1 comprising the amino acid sequence of SEQ ID NO:74, an HVR-L2 comprising the amino acid sequence of SEQ ID NO:75, and an HVR-L3 comprising the amino acid sequence of SEQ ID NO:76. In some embodiments, the antibody comprises a heavy chain variable (VH) region and a light chain variable (VL) region; wherein the VH region comprises an HVR-H1 comprising the amino acid sequence of SEQ ID NO:53, an HVR-H2 comprising the amino acid sequence of SEQ ID NO:54, and an HVR-H3 comprising the amino acid sequence of SEQ ID NO:55; and wherein the VL region comprises an HVR-L1 comprising the amino acid sequence of SEQ ID NO:56, an HVR-L2 comprising the amino acid sequence of SEQ ID NO:57, and an HVR-L3 comprising the amino acid sequence of SEQ ID NO:58. In some embodiments, the antibody comprises a heavy chain variable (VH) region and a light chain variable (VL) region; wherein the VH region comprises an HVR-H1 comprising the amino acid sequence of SEQ ID NO:29, an HVR-H2 comprising the amino acid sequence of SEQ ID NO:30, and an HVR-H3 comprising the amino acid sequence of SEQ ID NO:31; and wherein the VL region comprises an HVR-L1 comprising the amino acid sequence of SEQ ID NO:32, an HVR-L2 comprising the amino acid sequence of SEQ ID NO:33, and an HVR-L3 comprising the amino acid sequence of SEQ ID NO:34. In some embodiments, the antibody comprises a heavy chain variable (VH) region and a light chain variable (VL) region; wherein the VH region comprises an HVR-H1 comprising the amino acid sequence of SEQ ID NO:23, an HVR-H2 comprising the amino acid sequence of SEQ ID NO:24, and an HVR-H3 comprising the amino acid sequence of SEQ ID NO:25; and wherein the VL region comprises an HVR-L1 comprising the amino acid sequence of SEQ ID NO:26, an HVR-L2 comprising the amino acid sequence of SEQ ID NO:27, and an HVR-L3 comprising the amino acid sequence of SEQ ID NO:28. In some embodiments, the antibody comprises a heavy chain variable (VH) region and a light chain variable (VL) region; wherein the VH region comprises an HVR-H1 comprising the amino acid sequence of SEQ ID NO:35, an HVR-H2 comprising the amino acid sequence of SEQ ID NO:36, and an HVR-H3 comprising the amino acid sequence of SEQ ID NO:37; and wherein the VL region comprises an HVR-L1 comprising the amino acid sequence of SEQ ID NO:38, an HVR-L2 comprising the amino acid sequence of SEQ ID NO:39, and an HVR-L3 comprising the amino acid sequence of SEQ ID NO:40. In some embodiments, the antibody comprises a heavy chain variable (VH) region and a light chain variable (VL) region; wherein the VH region comprises an HVR-H1 comprising the amino acid sequence of SEQ ID NO:41, an HVR-H2 comprising the amino acid sequence of SEQ ID NO:42, and an HVR-H3 comprising the amino acid sequence of SEQ ID NO:43; and wherein the VL region comprises an HVR-L1 comprising the amino acid sequence of SEQ ID NO:44, an HVR-L2 comprising the amino acid sequence of SEQ ID NO:45, and an HVR-L3 comprising the amino acid sequence of SEQ ID NO:46. In some embodiments, the antibody comprises a heavy chain variable (VH) region and a light chain variable (VL) region; wherein the VH region comprises an HVR-H1 comprising the amino acid sequence of SEQ ID NO:47, an HVR-H2 comprising the amino acid sequence of SEQ ID NO:48, and an HVR-H3 comprising the amino acid sequence of SEQ ID NO:49; and wherein the VL region comprises an HVR-L1 comprising the amino acid sequence of SEQ ID NO:50, an HVR-L2 comprising the amino acid sequence of SEQ ID NO:51, and an HVR-L3 comprising the amino acid sequence of SEQ ID NO:52. In some embodiments, the antibody comprises a heavy chain variable (VH) region and a light chain variable (VL) region; wherein the VH region comprises an HVR-H1 comprising the amino acid sequence of SEQ ID NO:59, an HVR-H2 comprising the amino acid sequence of SEQ ID NO:60, and an HVR-H3 comprising the amino acid sequence of SEQ ID NO:61; and wherein the VL region comprises an HVR-L1 comprising the amino acid sequence of SEQ ID NO:62, an HVR-L2 comprising the amino acid sequence of SEQ ID NO:63, and an HVR-L3 comprising the amino acid sequence of SEQ ID NO:64. In some embodiments, the antibody comprises a heavy chain variable (VH) region and a light chain variable (VL) region; wherein the VH region comprises an HVR-H1 comprising the amino acid sequence of SEQ ID NO:65, an HVR-H2 comprising the amino acid sequence of SEQ ID NO:66, and an HVR-H3 comprising the amino acid sequence of SEQ ID NO:67; and wherein the VL region comprises an HVR-L1 comprising the amino acid sequence of SEQ ID NO:68, an HVR-L2 comprising the amino acid sequence of SEQ ID NO:69, and an HVR-L3 comprising the amino acid sequence of SEQ ID NO:70. In some embodiments, the antibody comprises a heavy chain variable (VH) region and a light chain variable (VL) region; wherein the VH region comprises an HVR-H1 comprising the amino acid sequence of SEQ ID NO:135, an HVR-H2 comprising the amino acid sequence of SEQ ID NO:136, and an HVR-H3 comprising the amino acid sequence of SEQ ID NO:137; and wherein the VL region comprises an HVR-L1 comprising the amino acid sequence of SEQ ID NO:138, an HVR-L2 comprising the amino acid sequence of SEQ ID NO:139, and an HVR-L3 comprising the amino acid sequence of SEQ ID NO:140. In some embodiments, the antibody comprises a heavy chain variable (VH) region and a light chain variable (VL) region; wherein the VH region comprises an HVR-H1 comprising the amino acid sequence of SEQ ID NO:141, an HVR-H2 comprising the amino acid sequence of SEQ ID NO:142, and an HVR-H3 comprising the amino acid sequence of SEQ ID NO:143; and wherein the VL region comprises an HVR-L1 comprising the amino acid sequence of SEQ ID NO:144, an HVR-L2 comprising the amino acid sequence of SEQ ID NO:145, and an HVR-L3 comprising the amino acid sequence of SEQ ID NO:146. In some embodiments, the antibody comprises a heavy chain variable (VH) region and a light chain variable (VL) region; wherein the VH region comprises an HVR-H1 comprising the amino acid sequence of SEQ ID NO:147, an HVR-H2 comprising the amino acid sequence of SEQ ID NO:148, and an HVR-H3 comprising the amino acid sequence of SEQ ID NO:149; and wherein the VL region comprises an HVR-L1 comprising the amino acid sequence of SEQ ID NO:150, an HVR-L2 comprising the amino acid sequence of SEQ ID NO:151, and an HVR-L3 comprising the amino acid sequence of SEQ ID NO:152. In some embodiments, the antibody competes for binding to human Siglec-6 with a reference antibody that comprises a VH region comprising an HVR-H1 comprising the amino acid sequence of SEQ ID NO:5, an HVR-H2 comprising the amino acid sequence of SEQ ID NO:6, and an HVR-H3 comprising the amino acid sequence of SEQ ID NO:7 and a VL region comprising an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO:10. In some embodiments, the antibody competes for binding to human Siglec-6 with a reference antibody that comprises a VH region comprising an HVR-H1 comprising the amino acid sequence of SEQ ID NO:11, an HVR-H2 comprising the amino acid sequence of SEQ ID NO:12, and an HVR-H3 comprising the amino acid sequence of SEQ ID NO:13 and a VL region comprising an HVR-L1 comprising the amino acid sequence of SEQ ID NO:14, an HVR-L2 comprising the amino acid sequence of SEQ ID NO:15, and an HVR-L3 comprising the amino acid sequence of SEQ ID NO:16. In some embodiments, the antibody competes for binding to human Siglec-6 with a reference antibody that comprises a VH region comprising an HVR-H1 comprising the amino acid sequence of SEQ ID NO:17, an HVR-H2 comprising the amino acid sequence of SEQ ID NO:18, and an HVR-H3 comprising the amino acid sequence of SEQ ID NO:19 and a VL region comprising an HVR-L1 comprising the amino acid sequence of SEQ ID NO:20, an HVR-L2 comprising the amino acid sequence of SEQ ID NO:21, and an HVR-L3 comprising the amino acid sequence of SEQ ID NO:22. In some embodiments, the antibody competes for binding to human Siglec-6 with a reference antibody that comprises a VH region comprising an HVR-H1 comprising the amino acid sequence of SEQ ID NO:29, an HVR-H2 comprising the amino acid sequence of SEQ ID NO:30, and an HVR-H3 comprising the amino acid sequence of SEQ ID NO:31 and a VL region comprising an HVR-L1 comprising the amino acid sequence of SEQ ID NO:32, an HVR-L2 comprising the amino acid sequence of SEQ ID NO:33, and an HVR-L3 comprising the amino acid sequence of SEQ ID NO:34. In some embodiments, the antibody competes for binding to human Siglec-6 with a reference antibody that comprises a VH region comprising an HVR-H1 comprising the amino acid sequence of SEQ ID NO:23, an HVR-H2 comprising the amino acid sequence of SEQ ID NO:24, and an HVR-H3 comprising the amino acid sequence of SEQ ID NO:25 and a VL region comprising an HVR-L1 comprising the amino acid sequence of SEQ ID NO:26, an HVR-L2 comprising the amino acid sequence of SEQ ID NO:27, and an HVR-L3 comprising the amino acid sequence of SEQ ID NO:28. In some embodiments, the antibody competes for binding to human Siglec-6 with a reference antibody that comprises a VH region comprising an HVR-H1 comprising the amino acid sequence of SEQ ID NO:135, an HVR-H2 comprising the amino acid sequence of SEQ ID NO:136, and an HVR-H3 comprising the amino acid sequence of SEQ ID NO:137, and a VL region comprising an HVR-L1 comprising the amino acid sequence of SEQ ID NO:138, an HVR-L2 comprising the amino acid sequence of SEQ ID NO:139, and an HVR-L3 comprising the amino acid sequence of SEQ ID NO:140. In some embodiments, the antibody competes for binding to human Siglec-6 with a reference antibody that comprises a VH region comprising an HVR-H1 comprising the amino acid sequence of SEQ ID NO:141, an HVR-H2 comprising the amino acid sequence of SEQ ID NO:142, and an HVR-H3 comprising the amino acid sequence of SEQ ID NO:143, and a VL region comprising an HVR-L1 comprising the amino acid sequence of SEQ ID NO:144, an HVR-L2 comprising the amino acid sequence of SEQ ID NO:145, and an HVR-L3 comprising the amino acid sequence of SEQ ID NO:146. In some embodiments, the antibody competes for binding to human Siglec-6 with a reference antibody that comprises a VH region comprising an HVR-H1 comprising the amino acid sequence of SEQ ID NO:147, an HVR-H2 comprising the amino acid sequence of SEQ ID NO:148, and an HVR-H3 comprising the amino acid sequence of SEQ ID NO:149, and a VL region comprising an HVR-L1 comprising the amino acid sequence of SEQ ID NO:150, an HVR-L2 comprising the amino acid sequence of SEQ ID NO:151, and an HVR-L3 comprising the amino acid sequence of SEQ ID NO:152. In some embodiments, an anti-Siglec-6 antibody of the present disclosure comprises a VH domain comprising 1, 2, or all 3 CDR or HVR sequences present in the amino acid sequence of SEQ ID NO:105 and/or a VL domain comprising 1, 2, or all 3 CDR or HVR sequences present in the amino acid sequence of SEQ ID NO:106. In some embodiments, an anti-Siglec-6 antibody of the present disclosure comprises a VH domain comprising 1, 2, or all 3 CDR or HVR sequences present in the VH domain sequence of AK15 as described herein (see, e.g., Table 5) and/or a VL domain comprising 1, 2, or all 3 CDR or HVR sequences present in the VL domain sequence of AK15 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:107 and/or a VL domain comprising 1, 2, or all 3 CDR or HVR sequences present in the amino acid sequence of SEQ ID NO:108. In some embodiments, an anti-Siglec-6 antibody of the present disclosure comprises a VH domain comprising 1, 2, or all 3 CDR or HVR sequences present in the VH domain sequence of AK14 as described herein (see, e.g., Table 5) and/or a VL domain comprising 1, 2, or all 3 CDR or HVR sequences present in the VL domain sequence of AK14 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:109 and/or a VL domain comprising 1, 2, or all 3 CDR or HVR sequences present in the amino acid sequence of SEQ ID NO:110. In some embodiments, an anti-Siglec-6 antibody of the present disclosure comprises a VH domain comprising 1, 2, or all 3 CDR or HVR sequences present in the VH domain sequence of AK13 as described herein (see, e.g., Table 5) and/or a VL domain comprising 1, 2, or all 3 CDR or HVR sequences present in the VL domain sequence of AK13 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:111 and/or a VL domain comprising 1, 2, or all 3 CDR or HVR sequences present in the amino acid sequence of SEQ ID NO:112. In some embodiments, an anti-Siglec-6 antibody of the present disclosure comprises a VH domain comprising 1, 2, or all 3 CDR or HVR sequences present in the VH domain sequence of AK12 as described herein (see, e.g., Table 5) and/or a VL domain comprising 1, 2, or all 3 CDR or HVR sequences present in the VL domain sequence of AK12 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:113 and/or a VL domain comprising 1, 2, or all 3 CDR or HVR sequences present in the amino acid sequence of SEQ ID NO:114. In some embodiments, an anti-Siglec-6 antibody of the present disclosure comprises a VH domain comprising 1, 2, or all 3 CDR or HVR sequences present in the VH domain sequence of AK11 as described herein (see, e.g., Table 5) and/or a VL domain comprising 1, 2, or all 3 CDR or HVR sequences present in the VL domain sequence of AK11 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:115 and/or a VL domain comprising 1, 2, or all 3 CDR or HVR sequences present in the amino acid sequence of SEQ ID NO:116. In some embodiments, an anti-Siglec-6 antibody of the present disclosure comprises a VH domain comprising 1, 2, or all 3 CDR or HVR sequences present in the VH domain sequence of AK10 as described herein (see, e.g., Table 5) and/or a VL domain comprising 1, 2, or all 3 CDR or HVR sequences present in the VL domain sequence of AK10 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:117 and/or a VL domain comprising 1, 2, or all 3 CDR or HVR sequences present in the amino acid sequence of SEQ ID NO:118. In some embodiments, an anti-Siglec-6 antibody of the present disclosure comprises a VH domain comprising 1, 2, or all 3 CDR or HVR sequences present in the VH domain sequence of AK09 as described herein (see, e.g., Table 5) and/or a VL domain comprising 1, 2, or all 3 CDR or HVR sequences present in the VL domain sequence of AK09 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:119 and/or a VL domain comprising 1, 2, or all 3 CDR or HVR sequences present in the amino acid sequence of SEQ ID NO:120. In some embodiments, an anti-Siglec-6 antibody of the present disclosure comprises a VH domain comprising 1, 2, or all 3 CDR or HVR sequences present in the VH domain sequence of AK08 as described herein (see, e.g., Table 5) and/or a VL domain comprising 1, 2, or all 3 CDR or HVR sequences present in the VL domain sequence of AK08 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:121 and/or a VL domain comprising 1, 2, or all 3 CDR or HVR sequences present in the amino acid sequence of SEQ ID NO:122. In some embodiments, an anti-Siglec-6 antibody of the present disclosure comprises a VH domain comprising 1, 2, or all 3 CDR or HVR sequences present in the VH domain sequence of AK07 as described herein (see, e.g., Table 5) and/or a VL domain comprising 1, 2, or all 3 CDR or HVR sequences present in the VL domain sequence of AK07 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:123 and/or a VL domain comprising 1, 2, or all 3 CDR or HVR sequences present in the amino acid sequence of SEQ ID NO:124. In some embodiments, an anti-Siglec-6 antibody of the present disclosure comprises a VH domain comprising 1, 2, or all 3 CDR or HVR sequences present in the VH domain sequence of AK06 as described herein (see, e.g., Table 5) and/or a VL domain comprising 1, 2, or all 3 CDR or HVR sequences present in the VL domain sequence of AK06 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:125 and/or a VL domain comprising 1, 2, or all 3 CDR or HVR sequences present in the amino acid sequence of SEQ ID NO:126. In some embodiments, an anti-Siglec-6 antibody of the present disclosure comprises a VH domain comprising 1, 2, or all 3 CDR or HVR sequences present in the VH domain sequence of AK05 as described herein (see, e.g., Table 5) and/or a VL domain comprising 1, 2, or all 3 CDR or HVR sequences present in the VL domain sequence of AK05 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:127 and/or a VL domain comprising 1, 2, or all 3 CDR or HVR sequences present in the amino acid sequence of SEQ ID NO:128. In some embodiments, an anti-Siglec-6 antibody of the present disclosure comprises a VH domain comprising 1, 2, or all 3 CDR or HVR sequences present in the VH domain sequence of AK04 as described herein (see, e.g., Table 5) and/or a VL domain comprising 1, 2, or all 3 CDR or HVR sequences present in the VL domain sequence of AK04 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:129 and/or a VL domain comprising 1, 2, or all 3 CDR or HVR sequences present in the amino acid sequence of SEQ ID NO:130. In some embodiments, an anti-Siglec-6 antibody of the present disclosure comprises a VH domain comprising 1, 2, or all 3 CDR or HVR sequences present in the VH domain sequence of AK03 as described herein (see, e.g., Table 5) and/or a VL domain comprising 1, 2, or all 3 CDR or HVR sequences present in the VL domain sequence of AK03 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:131 and/or a VL domain comprising 1, 2, or all 3 CDR or HVR sequences present in the amino acid sequence of SEQ ID NO:132. In some embodiments, an anti-Siglec-6 antibody of the present disclosure comprises a VH domain comprising 1, 2, or all 3 CDR or HVR sequences present in the VH domain sequence of AK02 as described herein (see, e.g., Table 5) and/or a VL domain comprising 1, 2, or all 3 CDR or HVR sequences present in the VL domain sequence of AK02 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:133 and/or a VL domain comprising 1, 2, or all 3 CDR or HVR sequences present in the amino acid sequence of SEQ ID NO:134. In some embodiments, an anti-Siglec-6 antibody of the present disclosure comprises a VH domain comprising 1, 2, or all 3 CDR or HVR sequences present in the VH domain sequence of AK01 as described herein (see, e.g., Table 5) and/or a VL domain comprising 1, 2, or all 3 CDR or HVR sequences present in the VL domain sequence of AK01 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:153 and/or a VL domain comprising 1, 2, or all 3 CDR or HVR sequences present in the amino acid sequence of SEQ ID NO:154. In some embodiments, an anti-Siglec-6 antibody of the present disclosure comprises a VH domain comprising 1, 2, or all 3 CDR or HVR sequences present in the VH domain sequence of AK16 as described herein (see, e.g., Table 5) and/or a VL domain comprising 1, 2, or all 3 CDR or HVR sequences present in the VL domain sequence of AK16 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:155 and/or a VL domain comprising 1, 2, or all 3 CDR or HVR sequences present in the amino acid sequence of SEQ ID NO:156. In some embodiments, an anti-Siglec-6 antibody of the present disclosure comprises a VH domain comprising 1, 2, or all 3 CDR or HVR sequences present in the VH domain sequence of AK17 as described herein (see, e.g., Table 5) and/or a VL domain comprising 1, 2, or all 3 CDR or HVR sequences present in the VL domain sequence of AK17 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:157 and/or a VL domain comprising 1, 2, or all 3 CDR or HVR sequences present in the amino acid sequence of SEQ ID NO:158. In some embodiments, an anti-Siglec-6 antibody of the present disclosure comprises a VH domain comprising 1, 2, or all 3 CDR or HVR sequences present in the VH domain sequence of AK18 as described herein (see, e.g., Table 5) and/or a VL domain comprising 1, 2, or all 3 CDR or HVR sequences present in the VL domain sequence of AK18 as described herein (see, e.g., Table 5).

In some embodiments according to any of the embodiments described herein, the antibody comprises an Fc region. In some embodiments, the Fc region is a human Fc region. In some embodiments, the Fc region is a human IgG1, human IgG2, or human IgG4 Fc region. In some embodiments, the Fc region is a human IgG4 Fc region comprising the amino acid substitution S228P, numbering according to EU index. In some embodiments, the antibody comprises a heavy chain constant region comprising the amino acid sequence of SEQ ID NO:103. In some embodiments, the antibody comprises a heavy chain constant region comprising the amino acid sequence of SEQ ID NO:101 or 102. In some embodiments, the Fe region comprises one or more mutation(s) that reduce effector function. In some embodiments, the antibody comprises a human IgG1 Fc region with a substitution or deletion at one or more of the following position(s), numbering based on EU index: (a) L234 and/or L235; (b) A327, A330, and/or P331; (c) E233, L234, L235, and/or G236; (d) E233, L234, and/or L235; (e) E233, L234, L235, G236, A327, A330, and/or P331; (f) E233, L234, L235, A327, A330, and/or P331; (g) N297; (h) L242, N297, and/or K334; (i) A287, N297, and/or L306; (j) R292, N297, and/or V302; (k) N297, V323, and/or I332; (l) V259, N297, and/or L306; (m) L234, L235, K322, M252, S254, and/or T256; (n) L234, L235, and/or P329; or (o) L234, L235, and/or K322. In some embodiments, the antibody comprises a human IgG1 Fc region with one or more of the following mutation(s), numbering based on EU index: (a) L234A and/or L235A; (b) A327G, A330S, and/or P331S; (c) E233P, L234V, L235A, and/or G236del; (d) E233P, L234V, and/or L235A; (e) E233P, L234V, L235A, G236del, A327G, A330S, and/or P331S; (f) E233P, L234V, L235A, A327G, A330S, and/or P331S; (g) N297A; (h) N297G; (i) N297Q; (j) L242C, N297C, and/or K334C; (k) A287C, N297G, and/or L306C; (l) R292C, N297G, and/or V302C; (m) N297G, V323C, and/or I332C; (n) V259C, N297G, and/or L306C; (o) L234F, L235Q, K322Q, M252Y, S254T, and/or T256E; (p) L234A, L235A, and/or P329G; or (q) L234A, L235Q, and/or K322Q. In some embodiments, the antibody comprises a human IgG2 Fc region with a substitution or deletion at one or more of the following position(s), numbering based on EU index: (a) A330 and/or P331; (b) V234, G237, P238, H268, V309, A330, and/or P331; or (c) V234, G237, H268, V309, A330, P331, C232, C233, S267, L328, M252, S254, and/or T256. In some embodiments, the antibody comprises a human IgG2 Fc region with one or more of the following mutation(s), numbering based on EU index: (a) A330S and/or P331S; (b) V234A, G237A, P238S, H268A, V309L, A330S, and/or P331S; or (c) V234A, G237A, H268Q, V309L, A330S, P331S, C232S, C233S, S267E, L328F, M252Y, S254T, and/or T256E. In some embodiments, the antibody comprises a human IgG4 Fc region with a substitution or deletion at one or more of the following position(s), numbering based on EU index: (a) E233, F234, L235, and/or G236; (b) E233, F234, and/or L235; or (c) S228 and/or L235. In some embodiments, the antibody comprises a human IgG4 Fc region with one or more of the following mutation(s), numbering based on EU index: (a) E233P, F234V, L235A, and/or G236del; (b) E233P, F234V, and/or L235A; (c) S228P and/or L235E; or (d) S228P and/or L235A. In some embodiments, the Fc region comprises one or more mutation(s) that enhance effector function. In some embodiments, the antibody comprises a human IgG1 Fe region with a substitution or deletion at one or more of the following position(s), numbering based on EU index: (a) F243, R292, Y300, V305, and/or P396; (b) S239 and/or I332; (c) S239, I332, and/or A330; (d) S298, E333, and/or K334; (e) G236, S239, and/or I332; (f) K326 and/or E333; (g) S267, H268, and/or S324; or (h) E345, E430, and/or S440. In some embodiments, the antibody comprises a human IgG1 Fe region with one or more of the following mutation(s), numbering based on EU index: (a) F243L, R292P, Y300L, V305I, and/or P396L; (b) S239D and/or I332E; (c) S239D, I332E, and/or A330L; (d) S298A, E333A, and/or K334A; (e) G236A, S239D, and/or I332E; (f) K326W and/or E333S; (g) S267E, H268F, and/or S324T; or (h) E345R, E430G, and/or S440Y.

In some embodiments according to any of the embodiments described herein, at least one or two of the heavy chains of the antibody is non-fucosylated. In some embodiments, the antibody is produced in a cell line having an alpha-1,6-fucosyltransferase (Fut8) knockout. In some embodiments, the antibody is produced in a cell line overexpressing β1,4-N-acetylglucosaminyltransferase III (GnT-III). In some embodiments, the antibody is produced in a cell line overexpressing β1,4-N-acetylglucosaminyltransferase III (GnT-III) that additionally overexpresses Golgi μ-mannosidase II (ManII).

In some embodiments according to any of the embodiments described herein, the antibody is an antibody fragment selected from the group consisting of a Fab, F(ab′)2, Fab′-SH, Fv, and scFv fragment. In some embodiments, the antibody comprises a light chain constant (CL) domain. In some embodiments, the CL domain is a human kappa CL domain. In some embodiments, the light chain comprises the amino acid sequence of SEQ ID NO:104. In some embodiments, the antibody is a monoclonal antibody. In some embodiments, the antibody is a multispecific antibody. In some embodiments, the antibody is a bispecific antibody. In some embodiments, the antibody is conjugated to an agent. In some embodiments, the agent is a cytotoxic agent or label.

Other aspects of the present disclosure relate to compositions comprising the antibody according to any one of the above embodiments. In some embodiments, the antibody comprises a Fc region and N-glycoside-linked carbohydrate chains linked to the Fc region, wherein less than 50% of the N-glycoside-linked carbohydrate chains contain a fucose residue. In some embodiments, substantially none of the N-glycoside-linked carbohydrate chains contain a fucose residue.

Other aspects of the present disclosure relate to polynucleotides encoding the antibody according to any one of the above embodiments. Other aspects of the present disclosure relate to vectors comprising one or more polynucleotides encoding the antibody according to any one of the above embodiments. Other aspects of the present disclosure relate to host cells comprising the polynucleotide(s) and/or vector(s) according to any one of the above embodiments. In some embodiments, the host cell is a mammalian or insect cell. In some embodiments, the host cell is Chinese hamster ovary (CHO) cell. In some embodiments, the host cell comprises a Fut8 knockout. In some embodiments, the host cell overexpresses GnT-III. In some embodiments, the host cell overexpresses GnT-III and additionally overexpresses ManII. Other aspects of the present disclosure relate to methods of producing an antibody, comprising culturing the host cell according to any one of the above embodiments under a condition that produces the antibody. In some embodiments, the methods further comprise recovering the antibody produced by the host cell. Other aspects of the present disclosure relate to antibodies produced by the method according to any one of the above embodiments. Other aspects of the present disclosure relate to pharmaceutical compositions comprising the antibody according to any one of the above embodiments and a pharmaceutically acceptable carrier. Other aspects of the present disclosure relate to kits or articles of manufacture comprising a medicament comprising a composition or antibody according to any one of the above embodiments. In some embodiments, the kits or articles of manufacture further comprise a package insert comprising instructions for administration of the medicament in an individual in need thereof, e.g., according to any one of the methods disclosed herein.

Other aspects of the present disclosure relate to methods of treating a disease or condition characterized by increased activity and/or number of mast cells expressing Siglec-6 in a subject, comprising administering to the subject an effective amount of the antibody or composition according to any one of the above embodiments. Other aspects of the present disclosure relate to methods of inhibiting activation of mast cells expressing Siglec-6 in a subject in need thereof, comprising administering to the subject an effective amount of the antibody or composition according to any one of the above embodiments. Other aspects of the present disclosure relate to methods of depleting mast cells expressing Siglec-6 in a subject in need thereof, comprising administering to the subject an effective amount of the antibody or composition according to any one of the above embodiments. Other aspects of the present disclosure relate to uses of the antibody or composition according to any one of the above embodiments in the manufacture of a medicament, e.g., for treating a disease or condition characterized by increased activity and/or number of mast cells expressing Siglec-6, inhibiting activation of mast cells expressing Siglec-6, and/or depleting mast cells expressing Siglec-6, e.g., in a subject in need thereof. Other aspects of the present disclosure relate to the antibody or composition according to any one of the above embodiments for use as a medicament. Other aspects of the present disclosure relate to the antibody or composition according to any one of the above embodiments for use in treating a disease or condition characterized by increased activity and/or number of mast cells expressing Siglec-6, inhibiting activation of mast cells expressing Siglec-6, and/or depleting mast cells expressing Siglec-6, e.g., in a subject in need thereof.

In some embodiments according to any of the embodiments described herein, administration of the antibody or composition results in a decreased level of active tryptase in a sample obtained from the individual, as compared to a level of active tryptase in a sample obtained from the individual prior to the administration. In some embodiments, administration of the antibody or composition results in a decreased level of CCL2 in a sample obtained from the individual, as compared to a level of CCL2 in a sample obtained from the individual prior to the administration. In some embodiments, administration of the antibody or composition results in a decreased level of IL-13 in a sample obtained from the individual, as compared to a level of IL-13 in a sample obtained from the individual prior to the administration. In some embodiments, administration of the antibody or composition results in a decreased level of IL-8 in a sample obtained from the individual, as compared to a level of IL-8 in a sample obtained from the individual prior to the administration. In some embodiments, administration of the antibody or composition results in a decreased level of CCL4 in a sample obtained from the individual, as compared to a level of CCL4 in a sample obtained from the individual prior to the administration. In some embodiments, administration of the antibody or composition results in a decreased level of IL-6 in a sample obtained from the individual, as compared to a level of IL-6 in a sample obtained from the individual prior to the administration. In some embodiments, administration of the antibody or composition results in a decreased level of histamine in a sample obtained from the individual, as compared to a level of histamine in a sample obtained from the individual prior to the administration. In some embodiments, administration of the antibody or composition results in a decreased level of chymase in a sample obtained from the individual, as compared to a level of chymase in a sample obtained from the individual prior to the administration. In some embodiments, administration of the antibody or composition results in a decreased level of CPA3 in a sample obtained from the individual, as compared to a level of CPA3 in a sample obtained from the individual prior to the administration. In some embodiments, administration of the antibody or composition results in a decreased level of a prostaglandin or leukotriene in a sample obtained from the individual, as compared to a level of the prostaglandin or leukotriene in a sample obtained from the individual prior to the administration. In some embodiments, administration of the antibody or composition results in a decreased level of CD63, CD107a, CD203c, IgE, or MRGPRX2 in a sample (e.g., a biopsy sample) obtained from the individual, as compared to a level of the CD63, CD107a, CD203c, IgE, or MRGPRX2 in a sample obtained from the individual prior to the administration. In some embodiments, the sample is a biopsy sample. In some embodiments, the sample is a serum, plasma, or urine sample. In some embodiments, administration of the antibody or composition results in a decrease in one or more symptoms in the individual, as compared to the one or more symptoms in the individual prior to the administration. In some embodiments, the one or more symptoms are selected from the group consisting of nausea, cramping, constipation, abdominal pain, bloating, vomiting, diarrhea, fatigue, eye pain, light sensitivity, redness, discharge, runny nose, headache, dizziness, brain fog, itching, flushing, sweating, hives, hypotension, shortness of breath, bone pain, joint pain, weight loss, osteoporosis, angioedema, chest pain, anxiety, depression, rapid heartbeat, bronchoconstriction, and general pain. In some embodiments, the disease or condition is mastocytosis, mast cell leukemia, mast cell activation syndrome, gastroparesis, osteoporosis, osteopenia, renal osteodystrophy, bone fracture, Alzheimer's disease, chronic neuropathic pain, hyperalgesia, nonalcoholic fatty liver disease (NAFLD), nonalcoholic steatohepatitis (NASH), graft vs. host disease (GVH), colitis, hereditary alpha tryptasemia, neurofibroma, Kounis syndrome, urticaria, atopic dermatitis, contact dermatitis, angioedema, pruigo nodularis, cholangitis, psoriasis, irritable bowel syndrome (IBS), functional dyspepsia, asthma, allergy, keloid, chronic rhinosinusitis, aspirin exacerbated respiratory disease (AERD), chronic obstructive pulmonary disease (COPD), bullous pemphigoid, idiopathic pulmonary fibrosis, systemic sclerosis, interstitital cystitis, hidradenitis suppurativa, alopecia areata, vitiligo, mast cell gastrointestinal disease, Crohn's disease, rheumatoid arthritis, gastroesophageal reflux disease, viral infection, achalasia, postural tachycardia syndrome, amyotrophic lateral sclerosis (ALS), multiple sclerosis (MS), complex regional pain syndrome, or Ehlers-Danlos syndrome. In some embodiments, the individual has or has been diagnosed with mastocytosis, mast cell leukemia, mast cell activation syndrome, gastroparesis, osteoporosis, osteopenia, renal osteodystrophy, bone fracture, Alzheimer's disease, chronic neuropathic pain, hyperalgesia, nonalcoholic fatty liver disease (NAFLD), nonalcoholic steatohepatitis (NASH), graft vs. host disease (GVH), colitis, hereditary alpha tryptasemia, neurofibroma, Kounis syndrome, urticaria, atopic dermatitis, contact dermatitis, angioedema, pruigo nodularis, cholangitis, psoriasis, irritable bowel syndrome (IBS), functional dyspepsia, asthma, allergy, keloid, chronic rhinosinusitis, aspirin exacerbated respiratory disease (AERD), chronic obstructive pulmonary disease (COPD), bullous pemphigoid, idiopathic pulmonary fibrosis, systemic sclerosis, interstitital cystitis, hidradenitis suppurativa, alopecia areata, vitiligo, mast cell gastrointestinal disease, Crohn's disease, rheumatoid arthritis, gastroesophageal reflux disease, viral infection, achalasia, postural tachycardia syndrome, amyotrophic lateral sclerosis (ALS), multiple sclerosis (MS), complex regional pain syndrome, or Ehlers-Danlos syndrome.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

FIG. 1A shows that Siglec-6 is selectively expressed on human primary tissue mast cells, and not on other immune cells. MFI, median fluorescence intensity; NK, natural killer cells; DCs, dendritic cells.

FIG. 1B provides a schematic diagram of a human Siglec-6 protein.

FIG. 2 shows that anti-Siglec-6 monoclonal antibodies (mAbs) inhibit activation of human mast cells. Activation was assessed by flow cytometry using the activation marker CD63.

FIGS. 3A-3C show that anti-Siglec-6 mAbs inhibit production of cytokines, chemokines, and proteases from human mast cells. Shown are production of IL-8 (FIG. 3A), CCL4 (FIG. 3B), and active tryptase (FIG. 3C).

FIG. 4A shows that Siglec-6 mAb inhibition is dependent on epitope location and provides binning information for anti-Siglec-6 mAbs.

FIG. 4B shows inhibition of human mast cell activation by anti-Siglec-6 mAbs representing each bin shown in FIG. 4A.

FIGS. 5A & 5B show that certain anti-Siglec-6 mAbs, but not all anti-Siglec-6 mAbs, promote internalization of Siglec-6 on human mast cells.

FIG. 6 shows that the anti-Siglec-6 mAb AK04 requires Fc interaction to induce Siglec-6 internalization on human mast cells.

FIGS. 7A & 7B show that Siglec-6 mAbs AK04 and AK02 inhibit IL-13 production from activated human mast cells.

FIGS. 8A-8F show that Siglec-6 mAb inhibits systemic anaphylaxis and mast cell mediator production in vivo in humanized mice. Data are plotted as means+/−SD; n=6-7 mice/group.

FIG. 9 shows that Siglec-6 mAb AK04 displays antibody-dependent cellular phagocytosis (ADCP) activity against human tissue mast cells.

FIGS. 10A-10C show the results of epitope mapping for anti-Siglec-6 antibodies AK05 (FIG. 10A), AK04 (FIG. 10B), and MAB2859 (FIG. 10C). In FIGS. 10A-10C, the numbering of the amino acids determined to interact with the indicated anti-Siglec-6 antibodies is according to the Siglec-6 ECD sequence as shown in SEQ ID NO:1.

FIGS. 11A & 11B show that anti-Siglec-6 mAb treatment inhibited KIT (CD117)-mediated mast cell activation in vivo. CD63 expression on peritoneal mast cells was evaluated by flow cytometry (FIG. 11A, left panel). Cytokine and chemokine levels, including IL-6 (FIG. 11A, center panel), CCL2/MCP-1 (FIG. 11A, right panel), TNF (FIG. 11B, left panel), and CXCL1/GRO-α (FIG. 11B, right panel), were measured in peritoneal lavage by Meso Scale Discovery (MSD). * p<0.01; n=7-8 mice/group. For all panels, data from PBS treatment is shown at left, data from isotype mAb+mouse stem cell factor (SCF) treatment is shown at center, and data from anti-Siglec-6 mAb+SCF treatment is shown at right.

FIG. 11C shows that Siglec-6 interacts and colocalizes with CD117/KIT in mast cells (MCs). This interaction was evidenced by co-immunoprecipitation/Western blotting for Siglec-6 and CD117 (left), as well as colocalization by confocal microscopy using primary MCs (right).

FIG. 11D shows association between inhibitory phosphatase Shp-1 and Siglec-6 in mast cells using confocal microscopy with anti-Siglec-6 and anti-SHP-1 antibodies. The non-receptor inhibitory phosphatase Shp-1 was associated with Siglec-6 immunoreceptor tyrosine-based inhibitory motifs (ITIMs) upon phosphorylation. Arrows indicate colocalization between inhibitory phosphatase Shp-1 and Siglec-6.

FIG. 11E shows a model for mast cell inhibition through antibody-mediated engagement of Siglec-6.

FIGS. 12A & 12B show that anti-Siglec-6 mAb treatment inhibited skin inflammation in a model of hapten-induced contact dermatitis. Percent change in ear swelling was determined 24 hours after dinitrochlorobenzene (DNFB) challenge (FIG. 12A, left panel). Cytokine and chemokine levels (FIG. 12A, center and right panels, as indicated) were measured in ears cultured ex vivo for 3 hours by Meso Scale Discovery (MSD). Skin inflammation, as measured by counts of mast cells (FIG. 12B, left panel) and CD8+ T cells (FIG. 12B, right panel), was also measured. * p<0.01; n=6-7 mice/group. For all panels, data from sham treatment is shown at left, data from isotype mAb+DNFB (hapten) treatment is shown at center, and data from anti-Siglec-6 mAb+DNFB (hapten) treatment is shown at right.

FIG. 13 shows that anti-Siglec-6 mAb potently inhibited IgE-mediated human tissue mast cell activation (as measured by CD63 expression via flow cytometry).

FIG. 14 shows that reduction of human tissue mast cells in vivo was dependent on the Fc region of the anti-Siglec-6 mAb. Mast cells from the peritoneal cavity (left panel) and lung (right panel) of humanized mice (SGM3-BLT) were analyzed using flow cytometry. n=5-6 mice/group. For all panels, data from isotype control is shown at left, data from Siglec-6 mAb is shown at center, and data from anti-Siglec-6 F(ab′)2 is shown at right.

FIG. 15A provides a schematic timeline for testing activity of anti-Siglec-6 mAb in a mouse model of acute, IL-33-driven skin inflammation. ISO, isotype; S6, anti-Siglec-6 mAb.

FIGS. 15B-15D show that anti-Siglec-6 mAb treatment inhibited IL-33-mediated inflammation, as demonstrated by a significant reduction in the number of ear-infiltrating mast cells, neutrophils, and monocytes. Mast cell (FIG. 15B), neutrophil (FIG. 15C), and monocyte (FIG. 15D) numbers were reduced in the ears of the mice that were treated with anti-Siglec-6 mAb and had received intradermal injections of IL-33, but not PBS. * p<0.01; n=6-7 mice/group. For all panels, data from PBS control is shown at left, data from IL-33-treated isotype control is shown at center, and data from IL-33-treated anti-Siglec-6 mAb is shown at right.

FIG. 16A provides a schematic timeline for testing activity of anti-Siglec-6 mAb a mouse model of antigen-induced allergic gastrointestinal disease. OVA, ovalbumin; ISO, isotype; S6, anti-Siglec-6 mAb.

FIG. 16B shows that anti-Siglec-6 mAb inhibited allergic gastrointestinal disease in mice that were sensitized and subsequently challenged with ovalbumin peptide compared to isotype control. Stomach-infiltrating mast cells and eosinophils were reduced in antigen-stimulated mice given anti-Siglec-6 mAb compared to mice given isotype control, as was the level of CD63 expression on mast cells as a marker of activation. Mast cells from the stomach cavity of OVA-treated mice were analyzed for CD63 expression using flow cytometry. * p<0.01; n=6-7 mice/group. For all panels, data from PBS unstimulated control is shown at left, data from stimulated isotype control is shown at center, and data from stimulated anti-Siglec-6 mAb is shown at right. MC, mast cell; Eos, eosinophil.

FIG. 17 shows significantly reduced levels of cytokines and chemokines (IL-4, IL-13, MIP-2, and CCL4) in the serum of allergic mice treated with anti-Siglec-6 mAb compared to isotype control. * p<0.01; n=6-7 mice/group. For all panels, data from PBS unstimulated control is shown at left, data from stimulated isotype control is shown at center, and data from stimulated anti-Siglec-6 mAb is shown at right.

FIGS. 18A-18C show that anti-Siglec-6 mAb inhibits inflammation driven by the MRGPRX2 agonist LL37. FIG. 18A shows skin of mice treated with PBS alone (“PBS”), LL37 and isotype control antibody (“Isotype”), and LL37 and anti-Siglec-6 mAb (“Siglec-6”). Arrows indicate skin inflammation. FIG. 18B shows counts of eosinophils (upper left), monocytes (upper right), and mast cells (lower left) obtained from lesional skin in mice treated with PBS alone (left bar in all graphs), LL37 and isotype control antibody (middle bar in all graphs), or LL37 and anti-Siglec-6 mAb (right bar in all graphs). FIG. 18C shows counts of mast cells obtained from non-lesional skin in mice treated with PBS alone (left bar), LL37 and isotype control antibody (middle bar), or LL37 and anti-Siglec-6 mAb (right bar).

FIG. 19 shows the effect of Fc effector function on the ability of anti-Siglec-6 mAbs to inhibit mast cells. Mice were intravenously administered a single dose of humanized anti-Siglec-6 mAb (comprising a VH domain comprising the sequence of SEQ ID NO:161 and a VL domain comprising the sequence of SEQ ID NO:162) having an active mIgG2a Fc or inert mIgG2a Fc (LALA-PG mutations; L234A/L235A/P329G) at 1 mg/kg, 3 mg/kg, 10 mg/kg, or formulation buffer. On day 7, mice were sacrificed, and peritoneal mast cells were isolated followed by assessment of Siglec-6 receptor occupancy and mast cell activation. Siglec-6 receptor occupancy is shown in left panel. To assess mast cell inhibition, peritoneal mast cells were activated through the FcεRI pathway. Mast cell activation was assessed by quantifying the degranulation marker CD63 by flow cytometry. Mast cell activation is shown in right panel. In both panels, for each condition (Fc active vs. Fc inert), data are presented in the following order: formulation, 1 mg/kg, 3 mg/kg, 10 mg/kg (left to right).

### DETAILED DESCRIPTION

### I. Definitions

It is to be understood that the present disclosure is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising,” “consisting,” and “consisting essentially of” aspects and embodiments.

The term “antibody” includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules), as well as antibody fragments (e.g., Fab, F(ab′)₂, and Fv). The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, C T, 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the CH sequence and function, e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in the present disclosure. Common allotypic variants in human populations are those designated by the letters a, f, n, z.

An “isolated” antibody is one that has been identified, separated and/or recovered from a component of its production environment (e.g., naturally or recombinantly). In some embodiments, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In some embodiments, the polypeptide is purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or silver stain. Isolated antibody includes the antibody in situ within recombinant cells since at least one component of the antibody's natural environment will not be present. Ordinarily, however, an isolated polypeptide or antibody is prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. In some embodiments, monoclonal antibodies have a C-terminal cleavage at the heavy chain and/or light chain. For example, 1, 2, 3, 4, or 5 amino acid residues are cleaved at the C-terminus of heavy chain and/or light chain. In some embodiments, the C-terminal cleavage removes a C-terminal lysine from the heavy chain. In some embodiments, monoclonal antibodies have an N-terminal cleavage at the heavy chain and/or light chain. For example, 1, 2, 3, 4, or 5 amino acid residues are cleaved at the N-terminus of heavy chain and/or light chain. In some embodiments, monoclonal antibodies are highly specific, being directed against a single antigenic site. In some embodiments, monoclonal antibodies are highly specific, being directed against multiple antigenic sites (such as a bispecific antibody or a multispecific antibody). The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including, for example, the hybridoma method, recombinant DNA methods, phage-display technologies, and technologies for producing human or human-like antibodies in animals that have parts or all of the human immunoglobulin loci or genes encoding human immunoglobulin sequences.

The term “naked antibody” refers to an antibody that is not conjugated to a cytotoxic moiety or radiolabel.

The terms “full-length antibody,” “intact antibody” or “whole antibody” are used interchangeably to refer to an antibody in its substantially intact form, as opposed to an antibody fragment. Specifically whole antibodies include those with heavy and light chains including an Fe region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments.

Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. In some embodiments, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies of the present disclosure comprise a portion of an intact antibody, generally including the antigen binding or variable region of the intact antibody or the Fv region of an antibody which retains or has modified FcR binding capability. Examples of antibody fragments include linear antibody, single-chain antibody molecules and multispecific antibodies formed from antibody fragments.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used as a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. In some embodiments, the number of these amino acid substitutions in the FR are no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409. In some embodiments, humanized antibodies are directed against a single antigenic site. In some embodiments, humanized antibodies are directed against multiple antigenic sites. An alternative humanization method is described in U.S. Pat. No. 7,981,843 and U.S. Patent Application Publication No. 2006/0134098.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. Immunity 13:37-45 (2000); Johnson and Wu in Methods in Molecular Biology 248:1-25 (Lo, ed., Human Press, Totowa, N J, 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., Sequences of Proteins of Immunological Interest, 5^{th} Ed. Public Health Service, National Institute of Health, Bethesda, MD (1991)). Chothia HVRs refer instead to the location of the structural loops (Chothia and Lesk J. Mol. Biol. 196:901-917 (1987)). The “contact” HVRs are based on an analysis of the available complex crystal structures. The residues from each of these HVRs are noted below.

| Loop | Kabat | Chothia | Contact |
|---|---|---|---|
| L1 | L24-L34 | L26-L34 | L30-L36 |
| L2 | L50-L56 | L50-L56 | L46-L55 |
| L3 | L89-L97 | L91-L96 | L89-L96 |
| H1 | H31-H35B | H26-H32 | H30-H35B (Kabat Numbering) |
| H1 | H31-H35 | H26-H32 | H30-H35 (Chothia Numbering) |
| H2 | H50-H65 | H53-H56 | H47-H58 |
| H3 | H95-H102 | H95-H102 | H93-H101 |

Unless otherwise indicated, the variable-domain residues (HVR residues and framework region residues) are numbered according to Kabat et al., supra.

“Framework” or “FR” residues are those variable-domain residues other than the HVR residues as herein defined.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

An “acceptor human framework” for the purposes herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

An antibody that “binds to”, “specifically binds to” or is “specific for” a particular a polypeptide or an epitope on a particular polypeptide is one that binds to that particular polypeptide or epitope on a particular polypeptide without substantially binding to any other polypeptide or polypeptide epitope. In some embodiments, binding of an anti-Siglec-6 antibody described herein (e.g., an antibody that binds to human Siglec-6) to an unrelated non-Siglec-6 polypeptide is less than about 10% of the antibody binding to Siglec-6 as measured by methods known in the art (e.g., enzyme-linked immunosorbent assay (ELISA)). In some embodiments, an antibody that binds to a Siglec-6 (e.g., an antibody that binds to human Siglec-6) has a dissociation constant (Kd) of ≤1 μM, ≤100 nM, ≤10 nM, ≤2 nM, ≤1 nM, ≤0.7 nM, ≤0.6 nM, ≤0.5 nM, ≤0.1 nM, ≤0.01 nM, or ≤0.001 nM (e.g. 10⁻⁸M or less, e.g. from 10⁻⁸M to 10⁻¹³M, e.g., from 10⁻⁹ M to 10⁻¹³ M), about 250 pM or less, about 100 pM or less, about 10 pM or less, or about 1 pM.

The term “anti-Siglec-6 antibody” or “an antibody that binds to human Siglec-6” refers to an antibody that binds to a polypeptide or an epitope of human Siglec-6 without substantially binding to any other polypeptide or epitope of an unrelated non-Siglec-6 polypeptide.

The term “Siglec-6” as used herein refers to a human Siglec-6 protein. The term also includes naturally occurring variants of Siglec-6, including splice variants or allelic variants. Siglec-6, the sialic acid binding Ig-like lectin 6, is also known as CD327, CD33L, OBBP1, CD33L1, CD33L2, and CDW327. In some embodiments, a human Siglec-6 protein is any protein or polypeptide expressed by a human SIGLEC6 gene. An exemplary human SIGLEC6 gene is described by NCBI Ref. Seq. Gene ID No. 946. Amino acid sequences of exemplary human Siglec-6 proteins and domains thereof are described herein. For example, in some embodiments, a human Siglec-6 protein comprises an extracellular domain (ECD) comprising the amino acid sequence

(SEQ ID NO: 1)
QERRFQLEGPESLTVQEGLCVLVPCRLPTTLPASYYGYGYWFLEGADVP
VATNDPDEEVQEETRGRFHLLWDPRRKNCSLSIRDARRRDNAAYFFRLK
SKWMKYGYTSSKLSVRVMALTHRPNISIPGTLESGHPSNLTCSVPWVCE
QGTPPIFSWMSAAPTSLGPRTTQSSVLTITPRPQDHSTNLTCQVTFPGA
GVTMERTIQLNVSYAPQKVAISIFQGNSAAFKILQNTSSLPVLEGQALR
LLCDADGNPPAHLSWFQGFPALNATPISNTGVLELPQVGSAEEGDFTCR
AQHPLGSLQISLSLFVHWKPEGRAGGV.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptors); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991). In some embodiments, an anti-Siglec-6 antibody (e.g., an antibody that binds to human Siglec-6) described herein enhances ADCC. To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998). Other Fc variants that alter ADCC activity and other antibody properties include those disclosed by Ghetie et al., Nat Biotech. 15:637-40, 1997; Duncan et al, Nature 332:563-564, 1988; Lund et al., J. Immunol 147:2657-2662, 1991; Lund et al, Mol Immunol 29:53-59, 1992; Alegre et al, Transplantation 57:1537-1543, 1994; Hutchins et al., Proc Natl. Acad Sci USA 92:11980-11984, 1995; Jefferis et al, Immunol Lett. 44:111-117, 1995; Lund et al., FASEB J 9:115-119, 1995; Jefferis et al, Immunol Lett 54:101-104, 1996; Lund et al, J Immunol 157:4963-4969, 1996; Armour et al., Eur J Immunol 29:2613-2624, 1999; Idusogie et al, J Immunol 164:4178-4184, 200; Reddy et al, J Immunol 164:1925-1933, 2000; Xu et al., Cell Immunol 200:16-26, 2000; Idusogie et al, J Immunol 166:2571-2575, 2001; Shields et al., J Biol Chem 276:6591-6604, 2001; Jefferis et al, Immunol Lett 82:57-65. 2002; Presta et al., Biochem Soc Trans 30:487-490, 2002; Lazar et al., Proc. Natl. Acad. Sci. USA 103:4005-4010, 2006; U.S. Pat. Nos. 5,624,821; 5,885,573; 5,677,425; 6,165,745; 6,277,375; 5,869,046; 6,121,022; 5,624,821; 5,648,260; 6,194,551; 6,737,056; 6,821,505; 6,277,375; 7,335,742; and 7,317,091.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fe regions for use in the antibodies of the present disclosure include human IgG1, IgG2, IgG3 and IgG4. A single amino acid substitution (S228P according to Kabat numbering; designated IgG4Pro) may be introduced to abolish the heterogeneity observed in recombinant IgG4 antibody. See Angal, S. et al. (1993) Mol Immunol 30, 105-108.

“Non-fucosylated” or “fucose-deficient” antibody refers to a glycosylation antibody variant comprising an Fc region wherein a carbohydrate structure attached to the Fc region has reduced fucose or lacks fucose. In some embodiments, an antibody with reduced fucose or lacking fucose has improved ADCC function. Non-fucosylated or fucose-deficient antibodies have reduced fucose relative to the amount of fucose on the same antibody produced in a cell line. In some embodiments, a non-fucosylated or fucose-deficient antibody composition contemplated herein is a composition wherein less than about 50% of the N-linked glycans attached to the Fc region of the antibodies in the composition comprise fucose.

The terms “fucosylation” or “fucosylated” refers to the presence of fucose residues within the oligosaccharides attached to the peptide backbone of an antibody. Specifically, a fucosylated antibody comprises α (1,6)-linked fucose at the innermost N-acetylglucosamine (GlcNAc) residue in one or both of the N-linked oligosaccharides attached to the antibody Fc region, e.g. at position Asn 297 of the human IgG1 Fc domain (EU numbering of Fc region residues). Asn297 may also be located about +3 amino acids upstream or downstream of position 297, i.e. between positions 294 and 300, due to minor sequence variations in immunoglobulins.

The “degree of fucosylation” is the percentage of fucosylated oligosaccharides relative to all oligosaccharides identified by methods known in the art e.g., in an N-glycosidase F treated antibody composition assessed by matrix-assisted laser desorption-ionization time-of-flight mass spectrometry (MALDI-TOF MS). In a composition of a “fully fucosylated antibody” essentially all oligosaccharides comprise fucose residues, i.e. are fucosylated. In some embodiments, a composition of a fully fucosylated antibody has a degree of fucosylation of at least about 90%. Accordingly, an individual antibody in such a composition typically comprises fucose residues in each of the two N-linked oligosaccharides in the Fc region. Conversely, in a composition of a “fully non-fucosylated” antibody essentially none of the oligosaccharides are fucosylated, and an individual antibody in such a composition does not contain fucose residues in either of the two N-linked oligosaccharides in the Fc region. In some embodiments, a composition of a fully non-fucosylated antibody has a degree of fucosylation of less than about 10%. In a composition of a “partially fucosylated antibody” only part of the oligosaccharides comprise fucose. An individual antibody in such a composition can comprise fucose residues in none, one or both of the N-linked oligosaccharides in the Fc region, provided that the composition does not comprise essentially all individual antibodies that lack fucose residues in the N-linked oligosaccharides in the Fc region, nor essentially all individual antibodies that contain fucose residues in both of the N-linked oligosaccharides in the Fc region. In one embodiment, a composition of a partially fucosylated antibody has a degree of fucosylation of about 10% to about 80% (e.g., about 50% to about 80%, about 60% to about 80%, or about 70% to about 80%).

“Binding affinity” as used herein refers to the strength of the non-covalent interactions between a single binding site of a molecule (e.g., an antibody) and its binding partner (e.g., an antigen). In some embodiments, the binding affinity of an antibody for a Siglec-6 polypeptide or sub-domain thereof (e.g., the ECD, Domain 1, Domain 2, or Domain 3, e.g., as described herein) can generally be represented by a dissociation constant (Kd). Affinity can be measured by common methods known in the art, including those described herein.

“Binding avidity” as used herein refers to the binding strength of multiple binding sites of a molecule (e.g., an antibody) and its binding partner (e.g., an antigen).

An “isolated” nucleic acid molecule encoding the antibodies herein is a nucleic acid molecule that is identified and separated from at least one contaminant nucleic acid molecule with which it is ordinarily associated in the environment in which it was produced. In some embodiments, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “pharmaceutical formulation” refers to a preparation that is in such form as to permit the biological activity of the active ingredient to be effective, and that contains no additional components that are unacceptably toxic to an individual to which the formulation would be administered. Such formulations are sterile.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

As used herein, the term “treatment” or “treating” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disease (e.g., viral infection) are mitigated or eliminated. For example, an individual is successfully “treated” if treatment results in increasing the quality of life of those suffering from a disease, decreasing the dose of other medications required for treating the disease, reducing the frequency of recurrence of the disease, lessening severity of the disease, delaying the development or progression of the disease, and/or prolonging survival of individuals.

As used herein, “in conjunction with” or “in combination with” refers to administration of one treatment modality in addition to another treatment modality. As such, “in conjunction with” or “in combination with” refers to administration of one treatment modality before, during or after administration of the other treatment modality to the individual.

As used herein, the term “prevention” or “preventing” includes providing prophylaxis with respect to occurrence or recurrence of a disease in an individual. An individual may be predisposed to a disease, susceptible to a disease, or at risk of developing a disease, but has not yet been diagnosed with the disease.

An “effective amount” refers to at least an amount effective, at dosages and for periods of time necessary, to achieve the desired or indicated effect, including a therapeutic or prophylactic result. An effective amount can be provided in one or more administrations. A “therapeutically effective amount” is at least the minimum concentration required to effect a measurable improvement of a particular disease. A therapeutically effective amount herein may vary according to factors such as the disease state, age, sex, and weight of the patient, and the ability of the antibody to elicit a desired response in the individual. A therapeutically effective amount may also be one in which any toxic or detrimental effects of the antibody are outweighed by the therapeutically beneficial effects. A “prophylactically effective amount” refers to an amount effective, at the dosages and for periods of time necessary, to achieve the desired prophylactic result. Typically but not necessarily, since a prophylactic dose is used in individuals prior to or at the earlier stage of disease, the prophylactically effective amount can be less than the therapeutically effective amount.

“Chronic” administration refers to administration of the medicament(s) in a continuous as opposed to acute mode, so as to maintain the initial therapeutic effect (activity) for an extended period of time. “Intermittent” administration is treatment that is not consecutively done without interruption, but rather is cyclic in nature.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, an “individual” or a “subject” is a mammal. A “mammal” for purposes of treatment includes humans, domestic and farm animals, and zoo, sports, or pet animals, such as dogs, horses, rabbits, cattle, pigs, hamsters, gerbils, mice, ferrets, rats, cats, etc. In some embodiments, the individual or subject is a human.

### II. Anti-Siglec-6 Antibodies and Compositions

Certain aspects of the present disclosure relate to antibodies that bind to human Siglec-6, i.e., anti-Siglec-6 antibodies.

In some embodiments, the anti-Siglec-6 antibody is a humanized antibody that binds to Domain 1 of an extracellular domain of human Siglec-6, e.g., comprising the amino acid sequence

(SEQ ID NO: 2)
QERRFQLEGPESLTVQEGLCVLVPCRLPTTLPASYYGYGYWFLEGADVP
VATNDPDEEVQEETRGRFHLLWDPRRKNCSLSIRDARRRDNAAYFFRLK
SKWMKYGYTSSKLSVRVMALTHR.

In some embodiments, the anti-Siglec-6 antibody binds to Domain 2 of an extracellular domain of human Siglec-6, e.g., comprising the amino acid sequence PNISIPGTLESGHPSNLTCSVPWVCEQGTPPIFSWMSAAPTSLGPRTTQSSVLTITPRPQDH STNLTCQVTFPGAGVTMERTIQLNVSYA (SEQ ID NO:3). In some embodiments, the antibody is a humanized or human antibody.

In some embodiments, the anti-Siglec-6 antibody binds to Domain 3 of an extracellular domain of human Siglec-6, e.g., comprising the amino acid sequence PQKVAISIFQGNSAAFKILQNTSSLPVLEGQALRLLCDADGNPPAHLSWFQGFPALNATP ISNTGVLELPQVGSAEEGDFTCRAQHPLGSLQISLSLFVHWKPEGRAGGV (SEQ ID NO:4). In some embodiments, the antibody is a humanized or human antibody.

In some embodiments, the anti-Siglec-6 antibody comprises 1, 2, 3, 4, 5, or all 6 HVR sequences of a single anti-Siglec-6 antibody as set forth in Table 2. In some embodiments, the anti-Siglec-6 antibody comprises a VH region comprising 1, 2, or all 3 HVR sequences of a VH region of a single anti-Siglec-6 antibody as set forth in Table 2. In some embodiments, the anti-Siglec-6 antibody comprises a VL region comprising 1, 2, or all 3 HVR sequences of a VL region of a single anti-Siglec-6 antibody as set forth in Table 2.

TABLE 2
Anti-Siglec-6 antibody HVR sequences (HVRs shown
according to Kabat definition unless otherwise
specified).

| SEQ ID | Antibody | Sequence | |
|---|---|---|---|
| NO | Name | Description | Sequence |
| 5 | AK04 | HVR-H1 | GFSLTSYGVS |
| 6 | | HVR-H2 | VIWHDGSTSYHSTLIS |
| 7 | | HVR-H3 | DGYSGTFAY |
| 8 | | HVR-L1 | TASSSVSSSYLH |
| 9 | | HVR-L2 | STSILAS |
| 10 | | HVR-L3 | HQYHRSPYT |
| 11 | AK05 | HVR-H1 | GYIFTSYWMY |
| 12 | | HVR-H2 | NVNPYSGDTNYNEKFKT |
| 13 | | HVR-H3 | GDGLYYYAMDY |
| 14 | | HVR-L1 | KASQDINSYLT |
| 15 | | HVR-L2 | RASRLVD |
| 16 | | HVR-L3 | LQYDEFPLT |
| 17 | AK02 | HVR-H1 | GYTFTNYYIH |
| 18 | | HVR-H2 | MIHPISGSTNYNEKLKI |
| 19 | | HVR-H3 | RYYGSSYWYFDV |
| 20 | | HVR-L1 | RASSSVSYMN |
| 21 | | HVR-L2 | ATSNLAS |
| 22 | | HVR-L3 | QQWSSHPPT |
| 23 | AK14 | HVR-H1 | GYTFTTYGMS |
| 24 | | HVR-H2 | WINTYSGVPTYADDFKG |
| 25 | | HVR-H3 | LAYFGYYFDF |
| 26 | | HVR-L1 | KASQNVGSAVA |
| 27 | | HVR-L2 | SASNRYT |
| 28 | | HVR-L3 | QQYSSYPYT |
| 29 | AK11 | HVR-H1 | GFSFSTYVMS |
| 30 | | HVR-H2 | SISGGGSNTYYPDSVKG |
| 31 | | HVR-H3 | REVVVYWYFDV |
| 32 | | HVR-L1 | RASENIYSNLA |
| 33 | | HVR-L2 | SAKNLAD |
| 34 | | HVR-L3 | QHFWGTPLT |
| 35 | AK15 | HVR-H1 | GFSLTSYGVS |
| 36 | | HVR-H2 | IIWGDGSTNYHSALIS |
| 37 | | HVR-H3 | GLSDWYFDV |
| 38 | | HVR-L1 | RASSSVSYMH |
| 39 | | HVR-L2 | ATSNLAS |
| 40 | | HVR-L3 | QQWSSNPYT |
| 41 | AK13 | HVR-H1 | GYTFTTYGMS |
| 42 | | HVR-H2 | WINTYSGVPTYADDFKG |
| 43 | | HVR-H3 | LAYFGYYFDF |
| 44 | | HVR-L1 | KASQNVGSAVT |
| 45 | | HVR-L2 | SASNRYT |
| 46 | | HVR-L3 | QQYSSYPYT |
| 47 | AK12 | HVR-H1 | GFSLTSYGVH |
| 48 | | HVR-H2 | VIWGGGSTDYNAGFIS |
| 49 | | HVR-H3 | ALSDWYFDV |
| 50 | | HVR-L1 | RASSSVSYMH |
| 51 | | HVR-L2 | ATSNLAS |
| 52 | | HVR-L3 | QQWSSNPLT |
| 53 | AK10 | HVR-H1 | GYTFTDYYMN |
| 54 | | HVR-H2 | DINPKIGDTIYNQKFKG |
| 55 | | HVR-H3 | WDYYGSSLFDY |
| 56 | | HVR-L1 | KASQDVTTAVA |
| 57 | | HVR-L2 | WASTRHT |
| 58 | | HVR-L3 | QQHYSTPYT |
| 59 | AK09 | HVR-H1 | GYTLTTYGMS |
| 60 | | HVR-H2 | WINTYSGVPTYADEFKG |
| 61 | | HVR-H3 | LGYYGYYFDY |
| 62 | | HVR-L1 | RASQSIRNNLH |
| 63 | | HVR-L2 | YIFQSIS |
| 64 | | HVR-L3 | QQSDSWPHVT |
| 65 | AK08 | HVR-H1 | GFSLTSYGVN |
| 66 | | HVR-H2 | VIWGDGSTNYHSALIS |
| 67 | | HVR-H3 | ALSDWYFDV |
| 68 | | HVR-L1 | RASSSVSYMH |
| 69 | | HVR-L2 | ATSNLAS |
| 70 | | HVR-L3 | QQWSSNPYT |
| 71 | AK07 | HVR-H1 | GYTFTDYKMH |
| 72 | | HVR-H2 | NINPNNGGTSYNQKFKG |
| 73 | | HVR-H3 | RYYGSSSSYFDY |
| 74 | | HVR-L1 | SVSSSVSYMH |
| 75 | | HVR-L2 | DTSKLAS |
| 76 | | HVR-L3 | QQWSSNPPT |
| 77 | AK06 | HVR-H1 | GFTFSDYGIH |
| 78 | | HVR-H2 | YITSVSSTIYYADTVKG |
| 79 | | HVR-H3 | LDWYFDV |
| 80 | | HVR-L1 | SANSSVSFMH |
| 81 | | HVR-L2 | DTSKLAS |
| 82 | | HVR-L3 | QQWSSNPLT |
| 83 | AK03 | HVR-H1 | GYTFTSYWMH |
| 84 | | HVR-H2 | TIYPGNSDSTYNQKFKG |
| 85 | | HVR-H3 | EGTVVANYYGLDY |
| 86 | | HVR-L1 | RASSSVSYIH |
| 87 | | HVR-L2 | ATSNLAS |
| 88 | | HVR-L3 | QQWSSKPPT |
| 89 | AK01 | HVR-H1 | GFSLTSYSVH |
| 90 | | HVR-H2 | VIWSGGSTDYNA VFIS |
| 91 | | HVR-H3 | YSYYTMDY |
| 92 | | HVR-L1 | RSSQSLVLDNGNTYLH |
| 93 | | HVR-L2 | KVSNRFS |
| 94 | | HVR-L3 | SQSTHVPFT |
| 135 | AK16 | HVR-H1 | GFNIKDDYMH |
| 136 | | HVR-H2 | WIDLENGDTEYASKFQG |
| 137 | | HVR-H3 | PIGTVVAEGFAY |
| 138 | | HVR-L1 | RASENIYSNLA |
| 139 | | HVR-L2 | AATNLAD |
| 140 | | HVR-L3 | QHFWGTPYT |
| 141 | AK17 | HVR-H1 | GYTFTDYKMH |
| 142 | | HVR-H2 | NINPNNGGTSYNQKFKG |
| 143 | | HVR-H3 | RYYGSSSSYFDY |
| 144 | | HVR-L1 | RASQSIGTSIH |
| 145 | | HVR-L2 | YASESIS |
| 146 | | HVR-L3 | QQSNSWPTT |
| 147 | AK18 | HVR-H1 | GFTFSDYGMH |
| 148 | | HVR-H2 | YISSDSSTIYYADTVKG |
| 149 | | HVR-H3 | TIYYGNYFDY |
| 150 | | HVR-L1 | SASSSVSSSSLY |
| 151 | | HVR-L2 | SMSNLAS |
| 152 | | HVR-L3 | QQWSSNPLT |
| 168 | AK19/ | HVR-H1 | GFSLTSY |
| 169 | AK20/ | HVR-H2 | WHDGS |
| 7 | AK21 | HVR-H3 | DGYSGTFAY |
| 8 | (Chothia) | HVR-L1 | TASSSVSSSYLH |
| 9 | | HVR-L2 | STSILAS |
| 10 | | HVR-L3 | HQYHRSPYT |

In some embodiments, the antibody comprises a heavy chain variable (VH) region and a light chain variable (VL) region; wherein the VH region comprises an HVR-H1 comprising the amino acid sequence of SEQ ID NO:89, an HVR-H2 comprising the amino acid sequence of SEQ ID NO:90, and an HVR-H3 comprising the amino acid sequence of SEQ ID NO:91; and wherein the VL region comprises an HVR-L1 comprising the amino acid sequence of SEQ ID NO:92, an HVR-L2 comprising the amino acid sequence of SEQ ID NO:93, and an HVR-L3 comprising the amino acid sequence of SEQ ID NO:94.

In some embodiments, the antibody comprises a heavy chain variable (VH) region and a light chain variable (VL) region; wherein the VH region comprises an HVR-H1 comprising the amino acid sequence of SEQ ID NO:5, an HVR-H2 comprising the amino acid sequence of SEQ ID NO:6, and an HVR-H3 comprising the amino acid sequence of SEQ ID NO:7; and wherein the VL region comprises an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO:10.

In some embodiments, the antibody comprises a heavy chain variable (VH) region and a light chain variable (VL) region; wherein the VH region comprises an HVR-H1 comprising the amino acid sequence of SEQ ID NO:83, an HVR-H2 comprising the amino acid sequence of SEQ ID NO:84, and an HVR-H3 comprising the amino acid sequence of SEQ ID NO:85; and wherein the VL region comprises an HVR-L1 comprising the amino acid sequence of SEQ ID NO:86, an HVR-L2 comprising the amino acid sequence of SEQ ID NO:87, and an HVR-L3 comprising the amino acid sequence of SEQ ID NO:88.

In some embodiments, the antibody comprises a heavy chain variable (VH) region and a light chain variable (VL) region; wherein the VH region comprises an HVR-H1 comprising the amino acid sequence of SEQ ID NO:11, an HVR-H2 comprising the amino acid sequence of SEQ ID NO:12, and an HVR-H3 comprising the amino acid sequence of SEQ ID NO:13; and wherein the VL region comprises an HVR-L1 comprising the amino acid sequence of SEQ ID NO:14, an HVR-L2 comprising the amino acid sequence of SEQ ID NO:15, and an HVR-L3 comprising the amino acid sequence of SEQ ID NO:16.

In some embodiments, the antibody comprises a heavy chain variable (VH) region and a light chain variable (VL) region; wherein the VH region comprises an HVR-H1 comprising the amino acid sequence of SEQ ID NO:77, an HVR-H2 comprising the amino acid sequence of SEQ ID NO:78, and an HVR-H3 comprising the amino acid sequence of SEQ ID NO:79; and wherein the VL region comprises an HVR-L1 comprising the amino acid sequence of SEQ ID NO:80, an HVR-L2 comprising the amino acid sequence of SEQ ID NO:81, and an HVR-L3 comprising the amino acid sequence of SEQ ID NO:82.

In some embodiments, the antibody comprises a heavy chain variable (VH) region and a light chain variable (VL) region; wherein the VH region comprises an HVR-H1 comprising the amino acid sequence of SEQ ID NO:17, an HVR-H2 comprising the amino acid sequence of SEQ ID NO:18, and an HVR-H3 comprising the amino acid sequence of SEQ ID NO:19; and wherein the VL region comprises an HVR-L1 comprising the amino acid sequence of SEQ ID NO:20, an HVR-L2 comprising the amino acid sequence of SEQ ID NO:21, and an HVR-L3 comprising the amino acid sequence of SEQ ID NO:22.

In some embodiments, the antibody comprises a heavy chain variable (VH) region and a light chain variable (VL) region; wherein the VH region comprises an HVR-H1 comprising the amino acid sequence of SEQ ID NO:71, an HVR-H2 comprising the amino acid sequence of SEQ ID NO:72, and an HVR-H3 comprising the amino acid sequence of SEQ ID NO:73; and wherein the VL region comprises an HVR-L1 comprising the amino acid sequence of SEQ ID NO:74, an HVR-L2 comprising the amino acid sequence of SEQ ID NO:75, and an HVR-L3 comprising the amino acid sequence of SEQ ID NO:76.

In some embodiments, the antibody comprises a heavy chain variable (VH) region and a light chain variable (VL) region; wherein the VH region comprises an HVR-H1 comprising the amino acid sequence of SEQ ID NO:53, an HVR-H2 comprising the amino acid sequence of SEQ ID NO:54, and an HVR-H3 comprising the amino acid sequence of SEQ ID NO:55; and wherein the VL region comprises an HVR-L1 comprising the amino acid sequence of SEQ ID NO:56, an HVR-L2 comprising the amino acid sequence of SEQ ID NO:57, and an HVR-L3 comprising the amino acid sequence of SEQ ID NO:58.

In some embodiments, the antibody comprises a heavy chain variable (VH) region and a light chain variable (VL) region; wherein the VH region comprises an HVR-H1 comprising the amino acid sequence of SEQ ID NO:29, an HVR-H2 comprising the amino acid sequence of SEQ ID NO:30, and an HVR-H3 comprising the amino acid sequence of SEQ ID NO:31; and wherein the VL region comprises an HVR-L1 comprising the amino acid sequence of SEQ ID NO:32, an HVR-L2 comprising the amino acid sequence of SEQ ID NO:33, and an HVR-L3 comprising the amino acid sequence of SEQ ID NO:34.

In some embodiments, the antibody comprises a heavy chain variable (VH) region and a light chain variable (VL) region; wherein the VH region comprises an HVR-H1 comprising the amino acid sequence of SEQ ID NO:23, an HVR-H2 comprising the amino acid sequence of SEQ ID NO:24, and an HVR-H3 comprising the amino acid sequence of SEQ ID NO:25; and wherein the VL region comprises an HVR-L1 comprising the amino acid sequence of SEQ ID NO:26, an HVR-L2 comprising the amino acid sequence of SEQ ID NO:27, and an HVR-L3 comprising the amino acid sequence of SEQ ID NO:28.

In some embodiments, the antibody comprises a heavy chain variable (VH) region and a light chain variable (VL) region; wherein the VH region comprises an HVR-H1 comprising the amino acid sequence of SEQ ID NO:35, an HVR-H2 comprising the amino acid sequence of SEQ ID NO:36, and an HVR-H3 comprising the amino acid sequence of SEQ ID NO:37; and wherein the VL region comprises an HVR-L1 comprising the amino acid sequence of SEQ ID NO:38, an HVR-L2 comprising the amino acid sequence of SEQ ID NO:39, and an HVR-L3 comprising the amino acid sequence of SEQ ID NO:40.

In some embodiments, the antibody comprises a heavy chain variable (VH) region and a light chain variable (VL) region; wherein the VH region comprises an HVR-H1 comprising the amino acid sequence of SEQ ID NO:41, an HVR-H2 comprising the amino acid sequence of SEQ ID NO:42, and an HVR-H3 comprising the amino acid sequence of SEQ ID NO:43; and wherein the VL region comprises an HVR-L1 comprising the amino acid sequence of SEQ ID NO:44, an HVR-L2 comprising the amino acid sequence of SEQ ID NO:45, and an HVR-L3 comprising the amino acid sequence of SEQ ID NO:46.

In some embodiments, the antibody comprises a heavy chain variable (VH) region and a light chain variable (VL) region; wherein the VH region comprises an HVR-H1 comprising the amino acid sequence of SEQ ID NO:47, an HVR-H2 comprising the amino acid sequence of SEQ ID NO:48, and an HVR-H3 comprising the amino acid sequence of SEQ ID NO:49; and wherein the VL region comprises an HVR-L1 comprising the amino acid sequence of SEQ ID NO:50, an HVR-L2 comprising the amino acid sequence of SEQ ID NO:51, and an HVR-L3 comprising the amino acid sequence of SEQ ID NO:52.

In some embodiments, the antibody comprises a heavy chain variable (VH) region and a light chain variable (VL) region; wherein the VH region comprises an HVR-H1 comprising the amino acid sequence of SEQ ID NO:59, an HVR-H2 comprising the amino acid sequence of SEQ ID NO:60, and an HVR-H3 comprising the amino acid sequence of SEQ ID NO:61; and wherein the VL region comprises an HVR-L1 comprising the amino acid sequence of SEQ ID NO:62, an HVR-L2 comprising the amino acid sequence of SEQ ID NO:63, and an HVR-L3 comprising the amino acid sequence of SEQ ID NO:64.

In some embodiments, the antibody comprises a heavy chain variable (VH) region and a light chain variable (VL) region; wherein the VH region comprises an HVR-H1 comprising the amino acid sequence of SEQ ID NO:65, an HVR-H2 comprising the amino acid sequence of SEQ ID NO:66, and an HVR-H3 comprising the amino acid sequence of SEQ ID NO:67; and wherein the VL region comprises an HVR-L1 comprising the amino acid sequence of SEQ ID NO:68, an HVR-L2 comprising the amino acid sequence of SEQ ID NO:69, and an HVR-L3 comprising the amino acid sequence of SEQ ID NO:70.

In some embodiments, the antibody comprises a heavy chain variable (VH) region and a light chain variable (VL) region; wherein the VH region comprises an HVR-H1 comprising the amino acid sequence of SEQ ID NO:135, an HVR-H2 comprising the amino acid sequence of SEQ ID NO:136, and an HVR-H3 comprising the amino acid sequence of SEQ ID NO:137; and wherein the VL region comprises an HVR-L1 comprising the amino acid sequence of SEQ ID NO:138, an HVR-L2 comprising the amino acid sequence of SEQ ID NO:139, and an HVR-L3 comprising the amino acid sequence of SEQ ID NO:140.

In some embodiments, the antibody comprises a heavy chain variable (VH) region and a light chain variable (VL) region; wherein the VH region comprises an HVR-H1 comprising the amino acid sequence of SEQ ID NO:141, an HVR-H2 comprising the amino acid sequence of SEQ ID NO:142, and an HVR-H3 comprising the amino acid sequence of SEQ ID NO:143; and wherein the VL region comprises an HVR-L1 comprising the amino acid sequence of SEQ ID NO:144, an HVR-L2 comprising the amino acid sequence of SEQ ID NO:145, and an HVR-L3 comprising the amino acid sequence of SEQ ID NO:146.

In some embodiments, the antibody comprises a heavy chain variable (VH) region and a light chain variable (VL) region; wherein the VH region comprises an HVR-H1 comprising the amino acid sequence of SEQ ID NO:147, an HVR-H2 comprising the amino acid sequence of SEQ ID NO:148, and an HVR-H3 comprising the amino acid sequence of SEQ ID NO:149; and wherein the VL region comprises an HVR-L1 comprising the amino acid sequence of SEQ ID NO:150, an HVR-L2 comprising the amino acid sequence of SEQ ID NO:151, and an HVR-L3 comprising the amino acid sequence of SEQ ID NO:152.

In some embodiments, an anti-Siglec-6 antibody provided herein competes for binding to human Siglec-6 (e.g., an ECD or sub-domain thereof of a human Siglec-6 protein) with a reference antibody, e.g., an anti-Siglec-6 antibody of the present disclosure. In some embodiments, an anti-Siglec-6 antibody provided herein competes for binding to human Siglec-6 (e.g., an ECD or sub-domain thereof of a human Siglec-6 protein) with one or more of the following anti-Siglec-6 antibodies described herein: AK04, AK05, AK02, AK14, AK11, AK15, AK13, AK12, AK10, AK09, AK08, AK07, AK06, AK03, AK01, AK16, AK17, and AK18. In some embodiments, an anti-Siglec-6 antibody provided herein competes for binding to Domain 1 of human Siglec-6 with one or more of the following anti-Siglec-6 antibodies described herein: AK04, AK05, AK02, AK07, AK06, AK03, and AK01. In some embodiments, an anti-Siglec-6 antibody provided herein competes for binding to Domain 2 of human Siglec-6 with one or more of the following anti-Siglec-6 antibodies described herein: AK10 and AK11. In some embodiments, an anti-Siglec-6 antibody provided herein competes for binding to Domain 3 of human Siglec-6 with one or more of the following anti-Siglec-6 antibodies described herein: AK09, AK08, AK12, AK13, AK14, and AK15. In some embodiments, the antibody competes for binding to human Siglec-6 with a reference antibody that comprises a VH region comprising an HVR-H1 comprising the amino acid sequence of SEQ ID NO:5, an HVR-H2 comprising the amino acid sequence of SEQ ID NO:6, and an HVR-H3 comprising the amino acid sequence of SEQ ID NO:7 and a VL region comprising an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO:10. In some embodiments, the antibody competes for binding to human Siglec-6 with a reference antibody that comprises a VH region comprising an HVR-H1 comprising the amino acid sequence of SEQ ID NO:11, an HVR-H2 comprising the amino acid sequence of SEQ ID NO:12, and an HVR-H3 comprising the amino acid sequence of SEQ ID NO:13 and a VL region comprising an HVR-L1 comprising the amino acid sequence of SEQ ID NO:14, an HVR-L2 comprising the amino acid sequence of SEQ ID NO:15, and an HVR-L3 comprising the amino acid sequence of SEQ ID NO:16. In some embodiments, the antibody competes for binding to human Siglec-6 with a reference antibody that comprises a VH region comprising an HVR-H1 comprising the amino acid sequence of SEQ ID NO:17, an HVR-H2 comprising the amino acid sequence of SEQ ID NO:18, and an HVR-H3 comprising the amino acid sequence of SEQ ID NO:19 and a VL region comprising an HVR-L1 comprising the amino acid sequence of SEQ ID NO:20, an HVR-L2 comprising the amino acid sequence of SEQ ID NO:21, and an HVR-L3 comprising the amino acid sequence of SEQ ID NO:22. In some embodiments, the antibody competes for binding to human Siglec-6 with a reference antibody that comprises a VH region comprising an HVR-H1 comprising the amino acid sequence of SEQ ID NO:29, an HVR-H2 comprising the amino acid sequence of SEQ ID NO:30, and an HVR-H3 comprising the amino acid sequence of SEQ ID NO:31 and a VL region comprising an HVR-L1 comprising the amino acid sequence of SEQ ID NO:32, an HVR-L2 comprising the amino acid sequence of SEQ ID NO:33, and an HVR-L3 comprising the amino acid sequence of SEQ ID NO:34. In some embodiments, the antibody competes for binding to human Siglec-6 with a reference antibody that comprises a VH region comprising an HVR-H1 comprising the amino acid sequence of SEQ ID NO:23, an HVR-H2 comprising the amino acid sequence of SEQ ID NO:24, and an HVR-H3 comprising the amino acid sequence of SEQ ID NO:25 and a VL region comprising an HVR-L1 comprising the amino acid sequence of SEQ ID NO:26, an HVR-L2 comprising the amino acid sequence of SEQ ID NO:27, and an HVR-L3 comprising the amino acid sequence of SEQ ID NO:28. In some embodiments, the antibody competes for binding to human Siglec-6 with a reference antibody that comprises a VH region comprising an HVR-H1 comprising the amino acid sequence of SEQ ID NO:135, an HVR-H2 comprising the amino acid sequence of SEQ ID NO:136, and an HVR-H3 comprising the amino acid sequence of SEQ ID NO:137, and a VL region comprising an HVR-L1 comprising the amino acid sequence of SEQ ID NO:138, an HVR-L2 comprising the amino acid sequence of SEQ ID NO:139, and an HVR-L3 comprising the amino acid sequence of SEQ ID NO:140. In some embodiments, the antibody competes for binding to human Siglec-6 with a reference antibody that comprises a VH region comprising an HVR-H1 comprising the amino acid sequence of SEQ ID NO:141, an HVR-H2 comprising the amino acid sequence of SEQ ID NO:142, and an HVR-H3 comprising the amino acid sequence of SEQ ID NO:143, and a VL region comprising an HVR-L1 comprising the amino acid sequence of SEQ ID NO:144, an HVR-L2 comprising the amino acid sequence of SEQ ID NO:145, and an HVR-L3 comprising the amino acid sequence of SEQ ID NO:146. In some embodiments, the antibody competes for binding to human Siglec-6 with a reference antibody that comprises a VH region comprising an HVR-H1 comprising the amino acid sequence of SEQ ID NO:147, an HVR-H2 comprising the amino acid sequence of SEQ ID NO:148, and an HVR-H3 comprising the amino acid sequence of SEQ ID NO:149, and a VL region comprising an HVR-L1 comprising the amino acid sequence of SEQ ID NO:150, an HVR-L2 comprising the amino acid sequence of SEQ ID NO:151, and an HVR-L3 comprising the amino acid sequence of SEQ ID NO:152.

In some embodiments, an anti-Siglec-6 antibody described herein binds to an extracellular domain (ECD) of a human Siglec-6 protein. In some embodiments, the Siglec-6 ECD comprises the amino acid sequence

(SEQ ID NO: 1)
QERRFQLEGPESLTVQEGLCVLVPCRLPTTLPASYYGYGYWFLEGADVP
VATNDPDEEVQEETRGRFHLLWDPRRKNCSLSIRDARRRDNAAYFFRLK
SKWMKYGYTSSKLSVRVMALTHRPNISIPGTLESGHPSNLTCSVPWVCE
QGTPPIFSWMSAAPTSLGPRTTQSSVLTITPRPQDHSTNLTCQVTFPGA
GVTMERTIQLNVSYAPQKVAISIFQGNSAAFKILQNTSSLPVLEGQALR
LLCDADGNPPAHLSWFQGFPALNATPISNTGVLELPQVGSAEEGDFTCR
AQHPLGSLQISLSLFVHWKPEGRAGGV.

In some embodiments, an anti-Siglec-6 antibody described herein binds to Domain 1, Domain 2, or Domain 3 of a human Siglec-6 protein (e.g., an ECD of a human Siglec-6 protein). In some embodiments, an anti-Siglec-6 antibody described herein binds to Domain 1 of a human Siglec-6 protein (e.g., an ECD of a human Siglec-6 protein). In some embodiments, Domain 1 comprises the amino acid sequence QERRFQLEGPESLTVQEGLCVLVPCRLPTTLPASYYGYGYWFLEGADVPVATNDPDEE VQEETRGRFHLLWDPRRKNCSLSIRDARRRDNAAYFFRLKSKWMKYGYTSSKLSVRV MALTHR (SEQ ID NO:2). In some embodiments, an anti-Siglec-6 antibody described herein binds to Domain 2 of a human Siglec-6 protein (e.g., an ECD of a human Siglec-6 protein). In some embodiments, Domain 2 comprises the amino acid sequence PNISIPGTLESGHPSNLTCSVPWVCEQGTPPIFSWMSAAPTSLGPRTTQSSVLTITPRPQDH STNLTCQVTFPGAGVTMERTIQLNVSYA (SEQ ID NO:3). In some embodiments, an anti-Siglec-6 antibody described herein binds to Domain 3 of a human Siglec-6 protein (e.g., an ECD of a human Siglec-6 protein). In some embodiments, Domain 3 comprises the amino acid sequence

(SEQ ID NO: 4)
PQKVAISIFQGNSAAFKILQNTSSLPVLEGQALRLLCDADGNPPAHLSW
FQGFPALNATPISNTGVLELPQVGSAEEGDFTCRAQHPLGSLQISLSLF
VHWKPEGRAGGV.

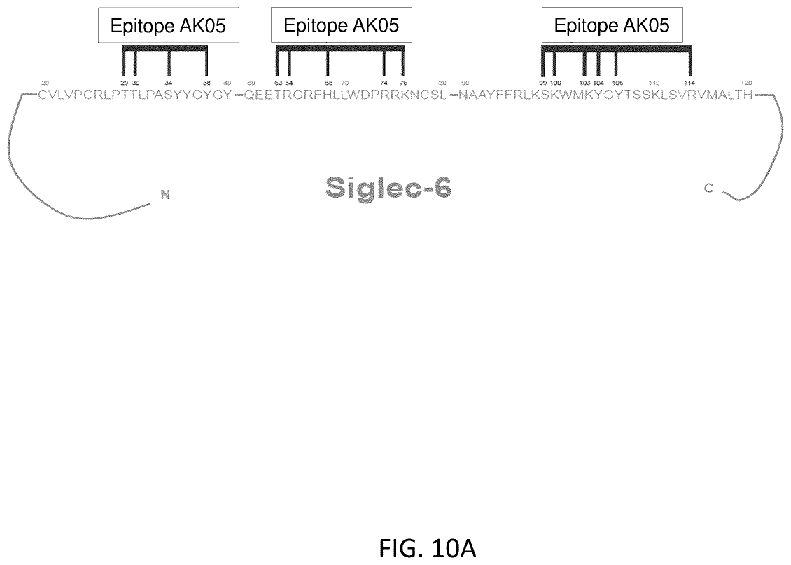

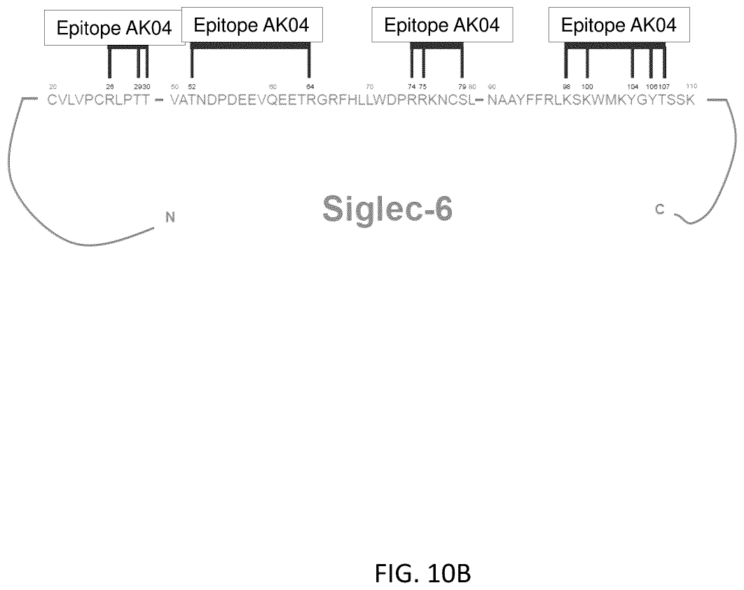

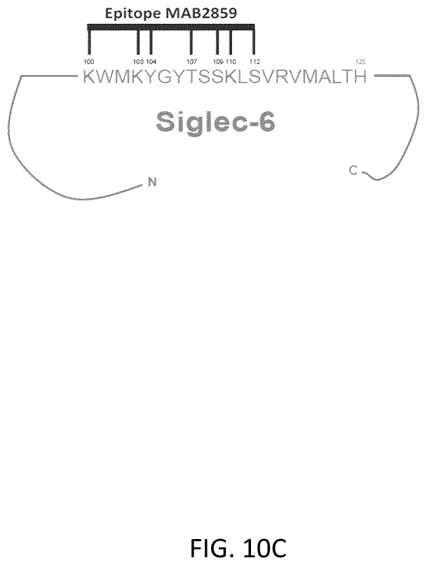

In some embodiments, an anti-Siglec-6 antibody provided herein binds the same epitope on human Siglec-6 (e.g., an ECD or sub-domain thereof of a human Siglec-6 protein) as an anti-Siglec-6 antibody of the present disclosure. In some embodiments, an anti-Siglec-6 antibody provided herein binds the same epitope on human Siglec-6 Domain 1, 2, or 3 as an anti-Siglec-6 antibody of the present disclosure. In some embodiments, an anti-Siglec-6 antibody provided herein binds the same epitope as AK05 (see, e.g., FIG. 10A). In some embodiments, an anti-Siglec-6 antibody provided herein binds the following amino acids on Siglec-6: 29, 30, 34, 38, 63, 64, 68, 74, 76, 99, 100, 103, 104, 106, and 114 (numbering according to the Siglec-6 ECD as shown in SEQ ID NO:1). In some embodiments, an anti-Siglec-6 antibody provided herein binds the same epitope as AK04 (see, e.g., FIG. 10B). In some embodiments, an anti-Siglec-6 antibody provided herein binds the following amino acids on Siglec-6: 26, 29, 30, 52, 64, 74, 75, 79, 98, 100, 104, 106, and 107 (numbering according to the Siglec-6 ECD as shown in SEQ ID NO: 1). Exemplary assays for epitope mapping are known in the art and exemplified herein. For example, epitope mapping can be performed using cross-linking mass spectrometry (XL-MS), e.g., as exemplified herein. Other assays include without limitation X-ray crystallography and alanine scanning mutagenesis.

TABLE 5
Variable domain sequences.

| SEQ ID | Antibody | Sequence | |
|---|---|---|---|
| NO | Name | Description | Sequence |
| 105 | AK15 | VH | QVQLKESGPGLVAPSQSLSITCTVSGFSLT |
| | | | SYGVSWVRQPPGKGLEWLGIIWGDGSTN |
| | | | YHSALISRLSISKDKSKSQVFLKLNSLQAD |
| | | | DTATYYCAKGLSDWYFDVWGTGTTVTV |
| | | | SS |
| 106 | | VL | QIVLSQSPAILSASPGEKVTMTCRASSSVS |
| | | | YMHWYQQKPGSSPKPWIYATSNLASGVP |
| | | | ARFSGSGSGTSYSLTISRVEAEDAATYYC |
| | | | QQWSSNPYTFGGGTKLEIK |
| 107 | AK14 | VH | QIQLVQSGPELKKPGETVKISCKASGYTFT |
| | | | TYGMSWVKQAPGKGLKWMGWINTYSG |
| | | | VPTYADDFKGRFAFSLETSATTAYLQINN |
| | | | LKNEDTATYFCARLAYFGYYFDFWGQGT |
| | | | TLTVSS |
| 108 | | VL | DIVMTQSQKFMSTTVGDRVSFTCKASQN |
| | | | VGSAVAWYQQKPGQSPKLLIYSASNRYT |
| | | | GVPDRFTGSGSGTDFTLTISNMQSEDLAD |
| | | | YFCQQYSSYPYTFGGGTKLEIK |
| 109 | AK13 | VH | QIQLVQSGPELKKPGETVKISCKASGYTFT |
| | | | TYGMSWVKQAPGKGLKWMGWINTYSG |
| | | | VPTYADDFKGRFAFSLETSATTAYLQINN |
| | | | LKNEDTATYFCARLAYFGYYFDFWGQGT |
| | | | TLTVSS |
| 110 | | VL | DIVMTQSQKFMSTTVGDRVSFTCKASQN |
| | | | VGSAVTWYQQKPGQSPKLLIYSASNRYT |
| | | | GVPDRFTGSGSGTDFTLTISNMQSEDLAD |
| | | | YFCQQYSSYPYTFGGGTKLEIK |
| 111 | AK12 | VH | QVQLKQSGPGLVQPSQSLSITCTVSGFSLT |
| | | | SYGVHWVRQSPGKGLEWLGVIWGGGST |
| | | | DYNAGFISRLSISKDNSKSQVFFKMNSLQ |
| | | | ADDTAIYYCARALSDWYFDVWGTGTTV |
| | | | TVSS |
| 112 | | VL | QIVLSQSPAILSASPGEKVTMTCRASSSVS |
| | | | YMHWYQQKPGSSPKPWIYATSNLASGVP |
| | | | ARFSGSGSGTSYSLTISRVEAEDAATYYC |
| | | | QQWSSNPLTFGAGTKLELK |
| 113 | AK11 | VH | EVMLVESGGGLVKPGGSLKLSCAASGFSF |
| | | | STYVMSWVRQTPEKRLEWVASISGGGSN |
| | | | TYYPDSVKGRFTISRDNAKNTLYLQMSSL |
| | | | RSEDTALYYCARREVVVYWYFDVWGTG |
| | | | TTVTVSS |
| 114 | | VL | DIQMTQSPASLSVSVGETVTITCRASENIY |
| | | | SNLAWYQQKQGKSPQLLVESAKNLADG |
| | | | VPSRFGGSGSGTQYSLKINSLQSEDFGTY |
| | | | YCQHFWGTPLTFGAGTKLELK |
| 115 | AK10 | VH | EVQLQQSGPELMKPGASVKISCKASGYTF |
| | | | TDYYMNWVKQSHGKSLEWIGDINPKIGD |
| | | | TIYNQKFKGKATLTVDKSSSTAYMELRSL |
| | | | TSEDSAVYYCARWDYYGSSLFDYWGQG |
| | | | TTLTVSS |
| 116 | | VL | DIVMTQSHKFMSTSVGDRVSITCKASQDV |
| | | | TTAVAWYQQKPGQSPKLLIYWASTRHTG |
| | | | VPDRFTGTGSGTDYTLTISNVQAEDLALY |
| | | | YCQQHYSTPYTFGGGTKLEIK |
| 117 | AK09 | VH | QIQLVQSGPELKKPGETVKISCKASGYTLT |
| | | | TYGMSWVKQAPGKGLKWMGWINTYSG |
| | | | VPTYADEFKGRFAFSLETSASTAYLQINN |
| | | | LKNEDTATYFCARLGYYGYYFDYWGQG |
| | | | TTLTVSS |
| 118 | | VL | DVVLTQSPATLSVTPGDSVSLSCRASQSIR |
| | | | NNLHWYQQKSHESPRLLIKYIFQSISGIPS |
| | | | RFSGSGSGTEFTLSINSVETEDFGMYFCQQ |
| | | | SDSWPHVTFGAGTKLELI |
| 119 | AK08 | VH | QVQLKESGPGLVAPSQSLSITCTVSGFSLT |
| | | | SYGVNWIRQPPGKGLEWLGVIWGDGSTN |
| | | | YHSALISRLSISKDNSKSQVFLKLNSLQTD |
| | | | DTATYYCAKALSDWYFDVWGTGTTVTV |
| | | | SS |
| 120 | | VL | QIVLSQSPAILSASPGEKVTMTCRASSSVS |
| | | | YMHWYQQKPGSSPKPWIYATSNLASGVP |
| | | | ARFSGSGSGTSYSLTISRVEAEDAATYYC |
| | | | QQWSSNPYTFGGGTKLEIK |
| 121 | AK07 | VH | EVQLQQSGPELVKPGASVKMSCKASGYT |
| | | | FTDYKMHWVKQSHGKSLEWIGNINPNNG |
| | | | GTSYNQKFKGKATLTVNKSSSTAYMELR |
| | | | SLTSEDSAVYYCARRYYGSSSSYFDYWG |
| | | | QGTTLTVSS |
| 122 | | VL | QIVLTQSPAIMSASPGEKVTMTCSVSSSVS |
| | | | YMHWYQQKSGTSPKRWIYDTSKLASGVP |
| | | | ARFSGSGSGTSYSLTISSMEAEDAATYYC |
| | | | QQWSSNPPTFGAGTKLELK |
| 123 | AK06 | VH | EVPLVESGGGLVKPGGSLKLSCAASGFTF |
| | | | SDYGIHWVRQAPEKGLEWVAYITSVSSTI |
| | | | YYADTVKGRFTISRDNAKNTLFLQMTSLR |
| | | | SEDTAMYYCAILDWYFDVWGTGTTVTVS |
| | | | S |
| 124 | | VL | QIVLTQSPAIMSASPGEKVTMTCSANSSVS |
| | | | FMHWFQQKSGTSPKRWIYDTSKLASGVP |
| | | | ARFSASGSGTSYSLTISSMEAEDAATYYC |
| | | | QQWSSNPLTFGAGTKVELK |
| 125 | AK05 | VH | QVQLQQPGTELVKPGASVQLSCKASGYIF |
| | | | TSYWMYWVKQRPGQGLEWIGNVNPYSG |
| | | | DTNYNEKFKTKATLTVDKASSTAFIQLSG |
| | | | LTSEDSAVYFCARGDGLYYYAMDYWGQ |
| | | | GTSVTVSS |
| 126 | | VL | DIKMTQSPSSMYASLGERVTITCKASQDI |
| | | | NSYLTWFQQKPGKSPKTLIYRASRLVDGV |
| | | | PSRFSGSGSGQDYSLTISSLEYEDMGIYYC |
| | | | LQYDEFPLTFGAGAKLELI |
| 127 | AK04 | VH | QVQLKESGPGLVAPSQSLSITCTVSGFSLT |
| | | | SYGVSWVRKPPGKGLEWLGVIWHDGSTS |
| | | | YHSTLISRLNISRDNSKSQVFLKLNSLQTD |
| | | | DTATYYCASDGYSGTFAYWGQGTLVTVS |
| | | | A |
| 128 | | VL | QIVVTQSPAIMSASLGERVTMTCTASSSVS |
| | | | SSYLHWYQQKPGSSPKLWIYSTSILASGV |
| | | | PARFSGSGSGTSYSLTVSSMEAEDAATYY |
| | | | CHQYHRSPYTFGGGSKLEIR |
| 129 | AK03 | VH | EVQLQQSGTVLARPGASVKMSCKTSGYT |
| | | | FTSYWMHWVKQRPGQGLEWIGTIYPGNS |
| | | | DSTYNQKFKGKAKLTAVTSASTAYMEVS |
| | | | SLTNEDSAVYYCSREGTVVANYYGLDY |
| | | | WGQGTSVTVSS |
| 130 | | VL | QIVLSQSPAILSASPGEKVTMTCRASSSVS |
| | | | YIHWYQQKPGSSPKPWIYATSNLASGVPA |
| | | | RFSGSGSGTSYSLTISRVEAEDAATYYCQ |
| | | | QWSSKPPTFGAGTKLELK |
| 131 | AK02 | VH | QVQLQQPGAELVKPGASVKLSCKASGYT |
| | | | FTNYYIHWVKQRPGQGLEWIGMIHPISGS |
| | | | TNYNEKLKIKATLTVDKSSSTAYMQLSSL |
| | | | TSEDSAVYYCARRYYGSSYWYFDVWGT |
| | | | GTTVTVSS |
| 132 | | VL | QIVLSQSPAILSASPGEKVTMTCRASSSVS |
| | | | YMNWYQQKPGSSPKPWIYATSNLASGVP |
| | | | ARFSGSGSGTPYSLTISRVEAEDAATYYC |
| | | | QQWSSHPPTFGSGTKLEIK |
| 133 | AK01 | VH | QVQLKQSGPGLVQPSQSLSITCTVSGFSLT |
| | | | SYSVHWVRQSPGKGLEWLGVIWSGGSTD |
| | | | YNAVFISRLSISRDNSKSQVFFKMNSLQA |
| | | | DDSAIYYCASYSYYTMDYWGQGTSVTVS |
| | | | S |
| 134 | | VL | DVVMTQTPLSLPVSLGDQASISCRSSQSL |
| | | | VLDNGNTYLHWYLQKPGQSPKLLIYKVS |
| | | | NRFSGVPDRFTGSGSGTDFTLKINRVEAE |
| | | | DLGVYFCSQSTHVPFTFGSGTKLEIK |
| 153 | AK16 | VH | EVQLQQSGAELVRPGASVKLSCSASGFNI |
| | | | KDDYMHWVKQRPGQGLEWIGWIDLENG |
| | | | DTEYASKFQGKATITADTSSNTAYLQLSS |
| | | | LTSEDTAVYYCTTPIGTVVAEGFAYWGQ |
| | | | GTLVTVSA |
| 154 | | VL | DIQMTQSPASLSVSVGETVTITCRASENIY |
| | | | SNLAWYQQKPGKSPQLLVYAATNLADG |
| | | | VPSRFSGSGSGTQYSLKINSLQSEDFGSYY |
| | | | CQHFWGTPYTFGGGTKLEIK |
| 155 | AK17 | VH | EVQLQQSGPELVKPGASVKMSCKASGYT |
| | | | FTDYKMHWVKQSHGKSLEWIGNINPNNG |
| | | | GTSYNQKFKGKATLTVNKSSSTAYMELR |
| | | | SLTSEDSAVYYCARRYYGSSSSYFDYWG |
| | | | QGTTLTVSS |
| 156 | | VL | DILLTQSPAILSVSPGERVSFSCRASQSIGT |
| | | | SIHWYQQRTNGSPRLLIKYASESISGIPSRF |
| | | | SGSGSGTDFTLSINSVESEDIADYYCQQSN |
| | | | SWPTTFGGGTKLEIK |
| 157 | AK18 | VH | EELLVESGGGLVKPGGSLKLSCAASGFTF |
| | | | SDYGMHWVRQAPEKGLEWVAYISSDSST |
| | | | IYYADTVKGRFTISRDNAKNTLFLQMTSL |
| | | | RSEDTAMYYCARTIYYGNYFDYWGQGT |
| | | | TLTVSS |
| 158 | | VL | QIVLTQSPAIMSASPGERVTMTCSASSSVS |
| | | | SSSLYWYQQKSGSSPKLWIYSMSNLASG |
| | | | VPARFSGSGSGTSYSLTINSMEAEDAATY |
| | | | YCQQWSSNPLTFGAGTKLELK |
| 159 | AK19 | VH | QVQLQESGPGLVKPSETLSLTCTVSGFSLT |
| | | | SYGVSWIRQPPGKGLEWIGVIWHDGSTSY |
| | | | HSTLISRVTISRDTSKNQVSLKLSSVTAAD |
| | | | TAVYYCASDGYSGTFAYWGQGTLVTVSS |
| 160 | | VL | QIVVTQSPATLSLSPGERATLSCTASSSVS |
| | | | SSYLHWYQQKPGQAPRLLIYSTSILASGIP |
| | | | ARFSGSGSGTDFTLTISSLQPEDFAVYYCH |
| | | | QYHRSPYTFGQGTKLEIK |
| 161 | AK20 | VH | QVQLQESGPGLVKPSETLSLTCTVSGFSLT |
| | | | SYGVSWIRQPPGKGLEWIGVIWHDGSTSY |
| | | | HPSLKSRVTISRDTSKNQVSLKLSSVTAA |
| | | | DTAVYYCASDGYSGTFAYWGQGTLVTV |
| | | | SS |
| 162 | | VL | QIVVTQSPATLSLSPGERATLSCTASSSVS |
| | | | SSYLHWYQQKPGQAPRLLIYSTSILASGIP |
| | | | ARFSGSGSGTDFTLTISSLQPEDFAVYYCH |
| | | | QYHRSPYTFGQGTKLEIK |
| 163 | AK21 | VH | QVQLQESGPGLVKPSETLSLTCTVSGFSLS |
| | | | SYGVSWIRQPPGKGLEWIGVIWHDGSTN |
| | | | YNPSLKSRVTISRDTSKNQVSLKLSSVTA |
| | | | ADTAVYYCASDGYSGTFAYWGQGTLVT |
| | | | VSS |
| 164 | | VL | QIVVTQSPATLSLSPGERATLSCTASSSVS |
| | | | SSYLHWYQQKPGQAPRLLIYSTSILASGIP |
| | | | ARFSGSGSGTDFTLTISSLQPEDFAVYYCH |
| | | | QYHRSPYTFGQGTKLEIK |

Many definitions for CDR or HVR sequences of an antibody variable domain are known in the art and may be used to describe an antibody of the present disclosure, e.g., by CDR/HVR sequences. In some embodiments, antibody CDR/HVR sequences are defined as in Kabat (see, e.g., Kabat et al., Sequences of Proteins of Immunological Interest, 5^{th} Ed. Public Health Service, National Institute of Health, Bethesda, MD (1991)). In some embodiments, antibody CDR/HVR sequences are defined as in Chothia (see, e.g., Chothia and Lesk J. Mol. Biol. 196:901-917 (1987)). For example, the humanized variable domain sequences of AK19, AK20, and AK21 listed in Table 5 above have their CDR sequences according to Chothia definition underlined. In some embodiments, an anti-Siglec-6 antibody of the present disclosure comprises a VH domain that comprises all 3 CDR sequences according to Chothia of AK19 as shown in Table 5 and a VL domain that comprises all 3 CDR sequences according to Chothia of AK19 as shown in Table 5. In some embodiments, an anti-Siglec-6 antibody of the present disclosure comprises a VH domain that comprises all 3 CDR sequences according to Chothia of AK20 as shown in Table 5 and a VL domain that comprises all 3 CDR sequences according to Chothia of AK20 as shown in Table 5. In some embodiments, an anti-Siglec-6 antibody of the present disclosure comprises a VH domain that comprises all 3 CDR sequences according to Chothia of AK21 as shown in Table 5 and a VL domain that comprises all 3 CDR sequences according to Chothia of AK21 as shown in Table 5.

Table 6 shows the CDR sequences of anti-Siglec-6 antibodies according to Kabat with the Chothia CDRs underlined. As shown in Table 6, the Kabat and Chothia definitions for CDRs CDR-H3, CDR-L1, CDR-L2, and CDR-L3 are identical, whereas the Chothia CDR-H1 is amino acids 1-7 of the corresponding Kabat CDR, and the Chothia CDR-H2 is amino acids 3-7 or 3-8 of the corresponding Kabat CDR. Therefore, in some embodiments, an anti-Siglec-6 antibody of the present disclosure comprises a VH domain that comprises an HVR-H1 comprising amino acids 1-7 of SEQ ID NO:5, an HVR-H2 comprising amino acids 3-7 of SEQ ID NO:6, an HVR-H3 comprising the amino acid sequence of SEQ ID NO:7; and a VL domain that comprises an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO:10. In some embodiments, an anti-Siglec-6 antibody of the present disclosure comprises a VH domain that comprises an HVR-H1 comprising amino acids 1-7 of SEQ ID NO:11, an HVR-H2 comprising amino acids 3-8 of SEQ ID NO:12, an HVR-H3 comprising the amino acid sequence of SEQ ID NO:13; and a VL domain that comprises an HVR-L1 comprising the amino acid sequence of SEQ ID NO:14, an HVR-L2 comprising the amino acid sequence of SEQ ID NO:15, and an HVR-L3 comprising the amino acid sequence of SEQ ID NO:16. In some embodiments, an anti-Siglec-6 antibody of the present disclosure comprises a VH domain that comprises an HVR-H1 comprising amino acids 1-7 of SEQ ID NO:17, an HVR-H2 comprising amino acids 3-8 of SEQ ID NO:18, an HVR-H3 comprising the amino acid sequence of SEQ ID NO:19; and a VL domain that comprises an HVR-L1 comprising the amino acid sequence of SEQ ID NO:20, an HVR-L2 comprising the amino acid sequence of SEQ ID NO:21, and an HVR-L3 comprising the amino acid sequence of SEQ ID NO:22. In some embodiments, an anti-Siglec-6 antibody of the present disclosure comprises a VH domain that comprises an HVR-H1 comprising amino acids 1-7 of SEQ ID NO:23, an HVR-H2 comprising amino acids 3-8 of SEQ ID NO:24, an HVR-H3 comprising the amino acid sequence of SEQ ID NO:25; and a VL domain that comprises an HVR-L1 comprising the amino acid sequence of SEQ ID NO:26, an HVR-L2 comprising the amino acid sequence of SEQ ID NO:27, and an HVR-L3 comprising the amino acid sequence of SEQ ID NO:28. In some embodiments, an anti-Siglec-6 antibody of the present disclosure comprises a VH domain that comprises an HVR-H1 comprising amino acids 1-7 of SEQ ID NO:29, an HVR-H2 comprising amino acids 3-8 of SEQ ID NO:30, an HVR-H3 comprising the amino acid sequence of SEQ ID NO:31; and a VL domain that comprises an HVR-L1 comprising the amino acid sequence of SEQ ID NO:32, an HVR-L2 comprising the amino acid sequence of SEQ ID NO:33, and an HVR-L3 comprising the amino acid sequence of SEQ ID NO:34. In some embodiments, an anti-Siglec-6 antibody of the present disclosure comprises a VH domain that comprises an HVR-H1 comprising amino acids 1-7 of SEQ ID NO:35, an HVR-H2 comprising amino acids 3-8 of SEQ ID NO:36, an HVR-H3 comprising the amino acid sequence of SEQ ID NO:37; and a VL domain that comprises an HVR-L1 comprising the amino acid sequence of SEQ ID NO:38, an HVR-L2 comprising the amino acid sequence of SEQ ID NO:39, and an HVR-L3 comprising the amino acid sequence of SEQ ID NO:40. In some embodiments, an anti-Siglec-6 antibody of the present disclosure comprises a VH domain that comprises an HVR-H1 comprising amino acids 1-7 of SEQ ID NO:41, an HVR-H2 comprising amino acids 3-8 of SEQ ID NO:42, an HVR-H3 comprising the amino acid sequence of SEQ ID NO:43; and a VL domain that comprises an HVR-L1 comprising the amino acid sequence of SEQ ID NO:44, an HVR-L2 comprising the amino acid sequence of SEQ ID NO:45, and an HVR-L3 comprising the amino acid sequence of SEQ ID NO:46. In some embodiments, an anti-Siglec-6 antibody of the present disclosure comprises a VH domain that comprises an HVR-H1 comprising amino acids 1-7 of SEQ ID NO:135, an HVR-H2 comprising amino acids 3-8 of SEQ ID NO:136, an HVR-H3 comprising the amino acid sequence of SEQ ID NO:137; and a VL domain that comprises an HVR-L1 comprising the amino acid sequence of SEQ ID NO:138, an HVR-L2 comprising the amino acid sequence of SEQ ID NO:139, and an HVR-L3 comprising the amino acid sequence of SEQ ID NO:140. In some embodiments, an anti-Siglec-6 antibody of the present disclosure comprises a VH domain that comprises an HVR-H1 comprising amino acids 1-7 of SEQ ID NO:47, an HVR-H2 comprising amino acids 3-8 of SEQ ID NO:48, an HVR-H3 comprising the amino acid sequence of SEQ ID NO:49; and a VL domain that comprises an HVR-L1 comprising the amino acid sequence of SEQ ID NO:50, an HVR-L2 comprising the amino acid sequence of SEQ ID NO:51, and an HVR-L3 comprising the amino acid sequence of SEQ ID NO:52. In some embodiments, an anti-Siglec-6 antibody of the present disclosure comprises a VH domain that comprises an HVR-H1 comprising amino acids 1-7 of SEQ ID NO:53, an HVR-H2 comprising amino acids 3-8 of SEQ ID NO:54, an HVR-H3 comprising the amino acid sequence of SEQ ID NO:55; and a VL domain that comprises an HVR-L1 comprising the amino acid sequence of SEQ ID NO:56, an HVR-L2 comprising the amino acid sequence of SEQ ID NO:57, and an HVR-L3 comprising the amino acid sequence of SEQ ID NO:58. In some embodiments, an anti-Siglec-6 antibody of the present disclosure comprises a VH domain that comprises an HVR-H1 comprising amino acids 1-7 of SEQ ID NO:59, an HVR-H2 comprising amino acids 3-8 of SEQ ID NO:60, an HVR-H3 comprising the amino acid sequence of SEQ ID NO:61; and a VL domain that comprises an HVR-L1 comprising the amino acid sequence of SEQ ID NO:62, an HVR-L2 comprising the amino acid sequence of SEQ ID NO:63, and an HVR-L3 comprising the amino acid sequence of SEQ ID NO:64. In some embodiments, an anti-Siglec-6 antibody of the present disclosure comprises a VH domain that comprises an HVR-H1 comprising amino acids 1-7 of SEQ ID NO:65, an HVR-H2 comprising amino acids 3-8 of SEQ ID NO:66, an HVR-H3 comprising the amino acid sequence of SEQ ID NO:67; and a VL domain that comprises an HVR-L1 comprising the amino acid sequence of SEQ ID NO:68, an HVR-L2 comprising the amino acid sequence of SEQ ID NO:69, and an HVR-L3 comprising the amino acid sequence of SEQ ID NO:70. In some embodiments, an anti-Siglec-6 antibody of the present disclosure comprises a VH domain that comprises an HVR-H1 comprising amino acids 1-7 of SEQ ID NO:141, an HVR-H2 comprising amino acids 3-8 of SEQ ID NO:142, an HVR-H3 comprising the amino acid sequence of SEQ ID NO:143; and a VL domain that comprises an HVR-L1 comprising the amino acid sequence of SEQ ID NO:144, an HVR-L2 comprising the amino acid sequence of SEQ ID NO:145, and an HVR-L3 comprising the amino acid sequence of SEQ ID NO:146. In some embodiments, an anti-Siglec-6 antibody of the present disclosure comprises a VH domain that comprises an HVR-H1 comprising amino acids 1-7 of SEQ ID NO:71, an HVR-H2 comprising amino acids 3-8 of SEQ ID NO:72, an HVR-H3 comprising the amino acid sequence of SEQ ID NO:73; and a VL domain that comprises an HVR-L1 comprising the amino acid sequence of SEQ ID NO:74, an HVR-L2 comprising the amino acid sequence of SEQ ID NO:75, and an HVR-L3 comprising the amino acid sequence of SEQ ID NO:76. In some embodiments, an anti-Siglec-6 antibody of the present disclosure comprises a VH domain that comprises an HVR-H1 comprising amino acids 1-7 of SEQ ID NO:77, an HVR-H2 comprising amino acids 3-8 of SEQ ID NO:78, an HVR-H3 comprising the amino acid sequence of SEQ ID NO:79; and a VL domain that comprises an HVR-L1 comprising the amino acid sequence of SEQ ID NO:80, an HVR-L2 comprising the amino acid sequence of SEQ ID NO:81, and an HVR-L3 comprising the amino acid sequence of SEQ ID NO:82. In some embodiments, an anti-Siglec-6 antibody of the present disclosure comprises a VH domain that comprises an HVR-H1 comprising amino acids 1-7 of SEQ ID NO:147, an HVR-H2 comprising amino acids 3-8 of SEQ ID NO:148, an HVR-H3 comprising the amino acid sequence of SEQ ID NO:149; and a VL domain that comprises an HVR-L1 comprising the amino acid sequence of SEQ ID NO:150, an HVR-L2 comprising the amino acid sequence of SEQ ID NO:151, and an HVR-L3 comprising the amino acid sequence of SEQ ID NO:152. In some embodiments, an anti-Siglec-6 antibody of the present disclosure comprises a VH domain that comprises an HVR-H1 comprising amino acids 1-7 of SEQ ID NO:83, an HVR-H2 comprising amino acids 3-8 of SEQ ID NO:84, an HVR-H3 comprising the amino acid sequence of SEQ ID NO:85; and a VL domain that comprises an HVR-L1 comprising the amino acid sequence of SEQ ID NO:86, an HVR-L2 comprising the amino acid sequence of SEQ ID NO:87, and an HVR-L3 comprising the amino acid sequence of SEQ ID NO:88. In some embodiments, an anti-Siglec-6 antibody of the present disclosure comprises a VH domain that comprises an HVR-H1 comprising amino acids 1-7 of SEQ ID NO:89, an HVR-H2 comprising amino acids 3-7 of SEQ ID NO:90, an HVR-H3 comprising the amino acid sequence of SEQ ID NO:91; and a VL domain that comprises an HVR-L1 comprising the amino acid sequence of SEQ ID NO:92, an HVR-L2 comprising the amino acid sequence of SEQ ID NO:93, and an HVR-L3 comprising the amino acid sequence of SEQ ID NO:94. In some embodiments, an anti-Siglec-6 antibody of the present disclosure comprises a VH domain that comprises all three CDR sequences of a single anti-Siglec-6 antibody according to Chothia as shown in Table 6 and a VL domain that comprises all three CDR sequences of the same anti-Siglec-6 antibody according to Chothia as shown in Table 6.

TABLE 6
CDRs as defined by Kabat, with Chothia CDRs underlined.

| Clone | CDR1 | CDR2 | CDR3 |
|---|---|---|---|
| AK04 | GFSLTSYGVS | VIWHDGSTSYHST | DGYSGTFAY |
| HC | (SEQ ID NO: 5) | LIS (SEQ ID NO: 6) | (SEQ ID NO: 7) |
| AK04 | TASSSVSSSYLH | STSILAS (SEQ ID | HQYHRSPYT |
| LC | (SEQ ID NO: 8) | NO: 9) | (SEQ ID |
| | | | NO: 10) |
| AK05 | GYIFTSYWMY | NVNPYSGDTNYN | GDGLYYYA |
| HC | (SEQ ID NO: 11) | EKFKT (SEQ ID | MDY (SEQ ID |
| | | NO: 12) | NO: 13) |
| AK05 | KASQDINSYLT | RASRLVD (SEQ ID | LQYDEFPLT |
| LC | (SEQ ID NO: 14) | NO: 15) | (SEQ ID |
| | | | NO: 16) |
| AK02 | GYTFTNYYIH | MIHPISGSTNYNEK | RYYGSSYWY |
| HC | (SEQ ID NO: 17) | LKI (SEQ ID NO: 18) | FDV (SEQ ID |
| | | | NO: 19) |
| AK02 | RASSSVSYMN | ATSNLAS (SEQ ID | QQWSSHPPT |
| LC | (SEQ ID NO: 20) | NO: 21) | (SEQ ID |
| | | | NO: 22) |
| AK14 | GYTFTTYGMS | WINTYSGVPTYAD | LAYFGYYFD |
| HC | (SEQ ID NO: 23) | DFKG (SEQ ID | F (SEQ ID |
| | | NO: 24) | NO: 25) |
| AK14 | KASQNVGSAVA | SASNRYT (SEQ ID | QQYSSYPYT |
| LC | (SEQ ID NO: 26) | NO: 27) | (SEQ ID |
| | | | NO: 28) |
| AK11 | GFSFSTYVMS | SISGGGSNTYYPDS | REVVVYWYF |
| HC | (SEQ ID NO: 29) | VKG (SEQ ID | DV (SEQ ID |
| | | NO: 30) | NO: 31) |
| AK11 | RASENIYSNLA | SAKNLAD (SEQ ID | QHFWGTPLT |
| LC | (SEQ ID NO: 32) | NO: 33) | (SEQ ID |
| | | | NO: 34) |
| AK15 | GFSLTSYGVS | IIWGDGSTNYHSA | GLSDWYFDV |
| HC | (SEQ ID NO: 35) | LIS (SEQ ID NO: 36) | (SEQ ID |
| | | | NO: 37) |
| AK15 | RASSSVSYMH | ATSNLAS (SEQ ID | QQWSSNPYT |
| LC | (SEQ ID NO: 38) | NO: 39) | (SEQ ID |
| | | | NO: 40) |
| AK13 | GYTFTTYGMS | WINTYSGVPTYAD | LAYFGYYFD |
| HC | (SEQ ID NO: 41) | DFKG (SEQ ID | F (SEQ ID |
| | | NO: 42) | NO: 43) |
| AK13 | KASQNVGSAVT | SASNRYT (SEQ ID | QQYSSYPYT |
| LC | (SEQ ID NO: 44) | NO: 45) | (SEQ ID |
| | | | NO: 46) |
| AK16 | GFNIKDDYMH | WIDLENGDTEYAS | PIGTVVAEGF |
| HC | (SEQ ID NO: 135) | KFQG (SEQ ID | AY (SEQ ID |
| | | NO: 136) | NO: 137) |
| AK16 | RASENIYSNLA | AATNLAD (SEQ ID | QHFWGTPYT |
| LC | (SEQ ID NO: 138) | NO: 139) | (SEQ ID |
| | | | NO: 140) |
| AK12 | GFSLTSYGVH | VIWGGGSTDYNA | ALSDWYFDV |
| HC | (SEQ ID NO: 47) | GFIS (SEQ ID | (SEQ ID |
| | | NO: 48) | NO: 49) |
| AK12 | RASSSVSYMH | ATSNLAS (SEQ ID | QQWSSNPLT |
| LC | (SEQ ID NO: 50) | NO: 51) | (SEQ ID |
| | | | NO: 52) |
| AK10 | GYTFTDYYMN | DINPKIGDTIYNQK | WDYYGSSLF |
| HC | (SEQ ID NO: 53) | FKG (SEQ ID | DY (SEQ ID |
| | | NO: 54) | NO: 55) |
| AK10 | KASQDVTTAVA | WASTRHT (SEQ ID | QQHYSTPYT |
| LC | (SEQ ID NO: 56) | NO: 57) | (SEQ ID |
| | | | NO: 58) |
| AK09 | GYTLTTYGMS | WINTYSGVPTYAD | LGYYGYYFD |
| HC | (SEQ ID NO: 59) | EFKG (SEQ ID | Y (SEQ ID |
| | | NO: 60) | NO: 61) |
| AK09 | RASQSIRNNLH | YIFQSIS (SEQ ID | QQSDSWPHV |
| LC | (SEQ ID NO: 62) | NO: 63) | T (SEQ ID |
| | | | NO: 64) |
| AK08 | GFSLTSYGVN | VIWGDGSTNYHSA | ALSDWYFDV |
| HC | (SEQ ID NO: 65) | LIS (SEQ ID NO: 66) | (SEQ ID |
| | | | NO: 67) |
| AK08 | RASSSVSYMH | ATSNLAS (SEQ ID | QQWSSNPYT |
| LC | (SEQ ID NO: 68) | NO: 69) | (SEQ ID |
| | | | NO: 70) |
| AK17 | GYTFTDYKMH | NINPNNGGTSYNQ | RYYGSSSSYF |
| HC | (SEQ ID NO: 141) | KFKG (SEQ ID | DY (SEQ ID |
| | | NO: 142) | NO: 143) |
| AK17 | RASQSIGTSIH | YASESIS (SEQ ID | QQSNSWPTT |
| LC | (SEQ ID NO: 144) | NO: 145) | (SEQ ID |
| | | | NO: 146) |
| AK07 | GYTFTDYKMH | NINPNNGGTSYNQ | RYYGSSSSYF |
| HC | (SEQ ID NO: 71) | KFKG (SEQ ID | DY (SEQ ID |
| | | NO: 72) | NO: 73) |
| AK07 | SVSSSVSYMH | DTSKLAS (SEQ ID | QQWSSNPPT |
| LC | (SEQ ID NO: 74) | NO: 75) | (SEQ ID |
| | | | NO: 76) |
| AK06 | GFTFSDYGIH | YITSVSSTIYYADT | LDWYFDV |
| HC | (SEQ ID NO: 77) | VKG (SEQ ID | (SEQ ID |
| | | NO: 78) | NO: 79) |
| AK06 | SANSSVSFMH | DTSKLAS (SEQ ID | QQWSSNPLT |
| LC | (SEQ ID NO: 80) | NO: 81) | (SEQ ID |
| | | | NO: 82) |
| AK18 | GFTFSDYGMH | YISSDSSTIYYADT | TIYYGNYFD |
| HC | (SEQ ID NO: 147) | VKG (SEQ ID | Y (SEQ ID |
| | | NO: 148) | NO: 149) |
| AK18 | SASSSVSSSSLY | SMSNLAS (SEQ ID | QQWSSNPLT |
| LC | (SEQ ID NO: 150) | NO: 151) | (SEQ ID |
| | | | NO: 152) |
| AK03 | GYTFTSYWMH | TIYPGNSDSTYNQ | EGTVVANYY |
| HC | (SEQ ID NO: 83) | KFKG (SEQ ID | GLDY (SEQ |
| | | NO: 84) | ID NO: 85) |
| AK03 | RASSSVSYIH | ATSNLAS (SEQ ID | QQWSSKPPT |
| LC | (SEQ ID NO: 86) | NO: 87) | (SEQ ID |
| | | | NO: 88) |
| AK01 | GFSLTSYSVH | VIWSGGSTDYNAV | YSYYTMDY |
| HC | (SEQ ID NO: 89) | FIS (SEQ ID NO: 90) | (SEQ ID |
| | | | NO: 91) |
| AK01 | RSSQSLVLDNGN | KVSNRFS (SEQ ID | SQSTHVPFT |
| LC | TYLH (SEQ ID | NO: 93) | (SEQ ID |
| | NO: 92) | | NO: 94) |

In some embodiments, antibody CDR/HVR sequences are defined as in IMGT (see, e.g., Lefranc, M. P. (1999) The Immunologist 7:132-136). In some embodiments, CDR/HVR sequences of a single antibody are defined as by mixing two or more definitions, e.g., Kabat, Chothia, and/or IMGT.

In some embodiments, an anti-Siglec-6 antibody of the present disclosure comprises a VH domain comprising 1, 2, or all 3 CDR or HVR sequences present in the amino acid sequence of SEQ ID NO:105 and/or a VL domain comprising 1, 2, or all 3 CDR or HVR sequences present in the amino acid sequence of SEQ ID NO:106. In some embodiments, an anti-Siglec-6 antibody of the present disclosure comprises a VH domain comprising 1, 2, or all 3 CDR or HVR sequences present in the VH domain sequence of AK15 as described herein (see, e.g., Table 5) and/or a VL domain comprising 1, 2, or all 3 CDR or HVR sequences present in the VL domain sequence of AK15 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:107 and/or a VL domain comprising 1, 2, or all 3 CDR or HVR sequences present in the amino acid sequence of SEQ ID NO:108. In some embodiments, an anti-Siglec-6 antibody of the present disclosure comprises a VH domain comprising 1, 2, or all 3 CDR or HVR sequences present in the VH domain sequence of AK14 as described herein (see, e.g., Table 5) and/or a VL domain comprising 1, 2, or all 3 CDR or HVR sequences present in the VL domain sequence of AK14 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:109 and/or a VL domain comprising 1, 2, or all 3 CDR or HVR sequences present in the amino acid sequence of SEQ ID NO:110. In some embodiments, an anti-Siglec-6 antibody of the present disclosure comprises a VH domain comprising 1, 2, or all 3 CDR or HVR sequences present in the VH domain sequence of AK13 as described herein (see, e.g., Table 5) and/or a VL domain comprising 1, 2, or all 3 CDR or HVR sequences present in the VL domain sequence of AK13 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:111 and/or a VL domain comprising 1, 2, or all 3 CDR or HVR sequences present in the amino acid sequence of SEQ ID NO:112. In some embodiments, an anti-Siglec-6 antibody of the present disclosure comprises a VH domain comprising 1, 2, or all 3 CDR or HVR sequences present in the VH domain sequence of AK12 as described herein (see, e.g., Table 5) and/or a VL domain comprising 1, 2, or all 3 CDR or HVR sequences present in the VL domain sequence of AK12 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:113 and/or a VL domain comprising 1, 2, or all 3 CDR or HVR sequences present in the amino acid sequence of SEQ ID NO:114. In some embodiments, an anti-Siglec-6 antibody of the present disclosure comprises a VH domain comprising 1, 2, or all 3 CDR or HVR sequences present in the VH domain sequence of AK11 as described herein (see, e.g., Table 5) and/or a VL domain comprising 1, 2, or all 3 CDR or HVR sequences present in the VL domain sequence of AK11 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:115 and/or a VL domain comprising 1, 2, or all 3 CDR or HVR sequences present in the amino acid sequence of SEQ ID NO:116. In some embodiments, an anti-Siglec-6 antibody of the present disclosure comprises a VH domain comprising 1, 2, or all 3 CDR or HVR sequences present in the VH domain sequence of AK10 as described herein (see, e.g., Table 5) and/or a VL domain comprising 1, 2, or all 3 CDR or HVR sequences present in the VL domain sequence of AK10 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:117 and/or a VL domain comprising 1, 2, or all 3 CDR or HVR sequences present in the amino acid sequence of SEQ ID NO:118. In some embodiments, an anti-Siglec-6 antibody of the present disclosure comprises a VH domain comprising 1, 2, or all 3 CDR or HVR sequences present in the VH domain sequence of AK09 as described herein (see, e.g., Table 5) and/or a VL domain comprising 1, 2, or all 3 CDR or HVR sequences present in the VL domain sequence of AK09 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:119 and/or a VL domain comprising 1, 2, or all 3 CDR or HVR sequences present in the amino acid sequence of SEQ ID NO:120. In some embodiments, an anti-Siglec-6 antibody of the present disclosure comprises a VH domain comprising 1, 2, or all 3 CDR or HVR sequences present in the VH domain sequence of AK08 as described herein (see, e.g., Table 5) and/or a VL domain comprising 1, 2, or all 3 CDR or HVR sequences present in the VL domain sequence of AK08 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:121 and/or a VL domain comprising 1, 2, or all 3 CDR or HVR sequences present in the amino acid sequence of SEQ ID NO:122. In some embodiments, an anti-Siglec-6 antibody of the present disclosure comprises a VH domain comprising 1, 2, or all 3 CDR or HVR sequences present in the VH domain sequence of AK07 as described herein (see, e.g., Table 5) and/or a VL domain comprising 1, 2, or all 3 CDR or HVR sequences present in the VL domain sequence of AK07 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:123 and/or a VL domain comprising 1, 2, or all 3 CDR or HVR sequences present in the amino acid sequence of SEQ ID NO:124. In some embodiments, an anti-Siglec-6 antibody of the present disclosure comprises a VH domain comprising 1, 2, or all 3 CDR or HVR sequences present in the VH domain sequence of AK06 as described herein (see, e.g., Table 5) and/or a VL domain comprising 1, 2, or all 3 CDR or HVR sequences present in the VL domain sequence of AK06 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:125 and/or a VL domain comprising 1, 2, or all 3 CDR or HVR sequences present in the amino acid sequence of SEQ ID NO:126. In some embodiments, an anti-Siglec-6 antibody of the present disclosure comprises a VH domain comprising 1, 2, or all 3 CDR or HVR sequences present in the VH domain sequence of AK05 as described herein (see, e.g., Table 5) and/or a VL domain comprising 1, 2, or all 3 CDR or HVR sequences present in the VL domain sequence of AK05 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:127 and/or a VL domain comprising 1, 2, or all 3 CDR or HVR sequences present in the amino acid sequence of SEQ ID NO:128. In some embodiments, an anti-Siglec-6 antibody of the present disclosure comprises a VH domain comprising 1, 2, or all 3 CDR or HVR sequences present in the VH domain sequence of AK04 as described herein (see, e.g., Table 5) and/or a VL domain comprising 1, 2, or all 3 CDR or HVR sequences present in the VL domain sequence of AK04 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:129 and/or a VL domain comprising 1, 2, or all 3 CDR or HVR sequences present in the amino acid sequence of SEQ ID NO:130. In some embodiments, an anti-Siglec-6 antibody of the present disclosure comprises a VH domain comprising 1, 2, or all 3 CDR or HVR sequences present in the VH domain sequence of AK03 as described herein (see, e.g., Table 5) and/or a VL domain comprising 1, 2, or all 3 CDR or HVR sequences present in the VL domain sequence of AK03 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:131 and/or a VL domain comprising 1, 2, or all 3 CDR or HVR sequences present in the amino acid sequence of SEQ ID NO:132. In some embodiments, an anti-Siglec-6 antibody of the present disclosure comprises a VH domain comprising 1, 2, or all 3 CDR or HVR sequences present in the VH domain sequence of AK02 as described herein (see, e.g., Table 5) and/or a VL domain comprising 1, 2, or all 3 CDR or HVR sequences present in the VL domain sequence of AK02 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:133 and/or a VL domain comprising 1, 2, or all 3 CDR or HVR sequences present in the amino acid sequence of SEQ ID NO:134. In some embodiments, an anti-Siglec-6 antibody of the present disclosure comprises a VH domain comprising 1, 2, or all 3 CDR or HVR sequences present in the VH domain sequence of AK01 as described herein (see, e.g., Table 5) and/or a VL domain comprising 1, 2, or all 3 CDR or HVR sequences present in the VL domain sequence of AK01 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:153 and/or a VL domain comprising 1, 2, or all 3 CDR or HVR sequences present in the amino acid sequence of SEQ ID NO:154. In some embodiments, an anti-Siglec-6 antibody of the present disclosure comprises a VH domain comprising 1, 2, or all 3 CDR or HVR sequences present in the VH domain sequence of AK16 as described herein (see, e.g., Table 5) and/or a VL domain comprising 1, 2, or all 3 CDR or HVR sequences present in the VL domain sequence of AK16 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:155 and/or a VL domain comprising 1, 2, or all 3 CDR or HVR sequences present in the amino acid sequence of SEQ ID NO:156. In some embodiments, an anti-Siglec-6 antibody of the present disclosure comprises a VH domain comprising 1, 2, or all 3 CDR or HVR sequences present in the VH domain sequence of AK17 as described herein (see, e.g., Table 5) and/or a VL domain comprising 1, 2, or all 3 CDR or HVR sequences present in the VL domain sequence of AK17 as described herein (see, e.g., Table 5). In some embodiments, an anti-Siglec-6 antibody of the present disclosure comprises a VH domain comprising 1, 2, or all 3 CDR or HVR sequences present in the amino acid sequence of SEQ ID NO:157 and/or a VL domain comprising 1, 2, or all 3 CDR or HVR sequences present in the amino acid sequence of SEQ ID NO:158. In some embodiments, an anti-Siglec-6 antibody of the present disclosure comprises a VH domain comprising 1, 2, or all 3 CDR or HVR sequences present in the VH domain sequence of AK18 as described herein (see, e.g., Table 5) and/or a VL domain comprising 1, 2, or all 3 CDR or HVR sequences present in the VL domain sequence of AK18 as described herein (see, e.g., Table 5). In some embodiments according to any of the embodiments described herein, the antibody is a humanized antibody.

In some embodiments, an anti-Siglec-6 antibody of the present disclosure comprises a VH region that comprises an HVR-H1 comprising the amino acid sequence of SEQ ID NO:168, an HVR-H2 comprising the amino acid sequence of SEQ ID NO:169, and an HVR-H3 comprising the amino acid sequence of SEQ ID NO:7; and/or a VL region that comprises an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO:10. In some embodiments, the antibody is a humanized antibody.

In some embodiments, an anti-Siglec-6 antibody of the present disclosure comprises a VH region that comprises the amino acid sequence of SEQ ID NO:159; and a VL region that comprises the amino acid sequence of SEQ ID NO:160. In some embodiments, an anti-Siglec-6 antibody of the present disclosure comprises a VH region that comprises the amino acid sequence of SEQ ID NO:161; and a VL region that comprises the amino acid sequence of SEQ ID NO:162. In some embodiments, an anti-Siglec-6 antibody of the present disclosure comprises a VH region that comprises the amino acid sequence of SEQ ID NO:163; and a VL region that comprises the amino acid sequence of SEQ ID NO:164.

In some embodiments, an anti-Siglec-6 antibody of the present disclosure binds to the extracellular domain of human Siglec-6 when expressed on a surface of a human mast cell. In some embodiments, binding of the antibody to the extracellular domain of human Siglec-6 inhibits activation of the mast cell. Assays for assessing mast cell activation are known in the art and exemplified herein. In some embodiments, binding of the antibody to the extracellular domain of human Siglec-6 inhibits CD63 expression, e.g., of a human mast cell. In some aspects, an anti-Siglec-6 antibody described herein inhibits one or more mast cell-mediated activities. In some embodiments, binding of the antibody to the extracellular domain of human Siglec-6 inhibits expression and/or release (e.g., by a human mast cell) of one or more of the following: IL-6, IL-8, IL-13, CCL2, CCL4, histamine, chymase, and tryptase (e.g., active tryptase). In some embodiments, binding of the antibody to the extracellular domain of human Siglec-6 inhibits expression and/or release (e.g., by a human mast cell) of one or more of the following inflammatory mediators: proteases (e.g., pan-tryptase, active or beta-tryptase, chymase, CPA3, heparin, etc.), leukotrienes (e.g., leukotriene C4 or B4, platelet activating factor, prostaglandin D2 or E2, etc.), amines (e.g., histamine, serotonin, dopamine, polyamines, etc.), growth factors (e.g., SCF, GM-CSFG-CSF, FGF, EGF, NGF, VEGF, PDGF, etc.), cytokines (e.g., TNF, IL-1b, IL-4, IL-5, IL-6, IL-8, IL-9, IL-10, IL-13, IL-17, IL-18, IL-31, IL-36, etc.), chemokines (e.g., CCL2, CCL3, CCL4, CCL5, CCL11, CCL12, CCL13, CCL24, CCL26, CXCL1, CXCL2, CXCL3, CXCL4, CXCL5, CXCL6, CXCL7, CXCL8, etc.), exosomes, and extracellular traps. For example, total and active tryptase as well as histamine, N-methyl histamine, and 11-beta-prostaglandin F2 can be measured in blood or urine to assess the reduction in mast cells. See, e.g., U.S. Patent Application Publication No. US 20110293631 for an exemplary mast cell activity assay.

In some embodiments, binding of the antibody to the extracellular domain of human Siglec-6 when expressed on a surface of a human mast cell leads to reduced levels of Siglec-6 on the cell surface (i.e., of the human mast cell). Binding of the antibody can lead to reduced levels of Siglec-6 on the cell membrane, e.g., through Siglec-6 internalization, endocytosis, shedding, cleaving, etc. Assays for assessing levels of Siglec-6 surface expression are known in the art and exemplified herein. In some embodiments, Siglec-6 surface expression is measured by flow cytometry, e.g., using an anti-Siglec-6 antibody with a detectable (e.g., fluorescent) tag. For example, as demonstrated herein, mast cells can be contacted with an anti-Siglec-6 antibody, and surface expression can be measured by flow cytometry with a fluorescent-tagged anti-Siglec-6 antibody that binds to a different epitope on Siglec-6 than the test antibody. Reduced fluorescence in the presence of the test antibody can indicate a reduced level of Siglec-6 surface expression. In some embodiments, binding of the antibody to the extracellular domain of human Siglec-6 when expressed on a surface of a human mast cell leads to reduced levels of Siglec-6 surface expression regardless of the presence/absence of an antibody Fc region, or regardless of the ability of the antibody Fc region to bind an Fc receptor (e.g., expressed on an effector cell). In some embodiments, binding of the antibody to the extracellular domain of human Siglec-6 when expressed on a surface of a human mast cell leads to an expression level Siglec-6 on the cell membrane that is reduced by at least about 10%, at least about 20%, at least about 30%, at least about 40%, at least about 50%, at least about 60%, at least about 70%, at least about 80%, at least about 90%, or at least about 100%, e.g., as compared to surface expression of Siglec-6 in the absence of an antibody, or in the absence of an antibody that does not bind to Siglec-6.

In other embodiments, binding of the antibody to the extracellular domain of human Siglec-6 when expressed on a surface of a human mast cell does not lead to reduced levels of Siglec-6 on the cell membrane (i.e., of the human mast cell). In some embodiments, binding of the antibody to the extracellular domain of human Siglec-6 when expressed on a surface of a human mast cell does not lead to reduced levels of Siglec-6 on the cell membrane regardless of the presence/absence of an antibody Fc region, or regardless of the ability of the antibody Fc region to bind an Fc receptor (e.g., expressed on an effector cell).

In some embodiments, binding of the antibody to the extracellular domain of human Siglec-6 when expressed on a surface of a human mast cell leads to reduced levels of Siglec-6 on the cell membrane (i.e., of the human mast cell) in the presence of a cell (e.g., an effector cell) expressing an Fc receptor. In some embodiments, the antibody comprises an Fc region (e.g., an active Fc region). In some embodiments, binding of the antibody to the extracellular domain of human Siglec-6 when expressed on a surface of a human mast cell leads to a reduced level of surface-expressed Siglec-6 dependent upon interaction between the antibody Fc region (e.g., an active Fc region) and an Fc receptor or an effector cell, e.g., that expresses an Fc receptor. In some embodiments, binding of the antibody to the extracellular domain of human Siglec-6 when expressed on a surface of a human mast cell leads to a reduced level of surface-expressed Siglec-6 only when the antibody is a full-length antibody comprising an Fc region (e.g., an active Fc region), e.g., in the presence of an Fc receptor or an effector cell (e.g., expressing an Fc receptor). In some embodiments, binding of the antibody to the extracellular domain of human Siglec-6 when expressed on a surface of a human mast cell leads to a reduced level of surface-expressed Siglec-6 when the antibody is an antibody fragment, e.g., lacking an Fc region. In some embodiments, binding of the antibody to the extracellular domain of human Siglec-6 when expressed on a surface of a human mast cell leads to a reduced level of surface-expressed Siglec-6 when the antibody is a full-length antibody comprising an Fc region that does not bind an Fc receptor (e.g., an inactive or dead Fc region lacking effector function). In some embodiments, binding of the antibody to the extracellular domain of human Siglec-6 when expressed on a surface of a human mast cell leads to a reduced level of surface-expressed Siglec-6 when the antibody is a full-length antibody comprising an Fc region (e.g., an active Fc region), e.g., in the presence of an Fc receptor or an effector cell (e.g., expressing an Fc receptor), but not when the antibody is an antibody fragment lacking an Fc region or comprising an Fc region that does not bind an Fc receptor (e.g., an inactive or dead Fc region). In some embodiments, binding of the antibody to the extracellular domain of human Siglec-6 when expressed on a surface of a human mast cell leads to an expression level Siglec-6 on the cell membrane that is reduced by at least about 10%, at least about 20%, at least about 30%, at least about 40%, at least about 50%, at least about 60%, at least about 70%, at least about 80%, at least about 90%, or at least about 100% when the antibody comprises an active Fc region, e.g., as compared to surface expression of Siglec-6 using an antibody that does not comprise an Fc region, or does not comprise an active Fc region.

In some embodiments, binding of the antibody to the extracellular domain of human Siglec-6 when expressed on a surface of a human mast cell induces dimerization and/or internalization of Siglec-6. In some embodiments, binding of the antibody to the extracellular domain of human Siglec-6 when expressed on a surface of a human mast cell induces dimerization and internalization of Siglec-6. In some embodiments, binding of the antibody to the extracellular domain of human Siglec-6 when expressed on a surface of a human mast cell induces endocytosis of Siglec-6. In some embodiments, binding of the antibody to the extracellular domain of human Siglec-6 when expressed on a surface of a human mast cell induces shedding of Siglec-6 (e.g., the Siglec-6 ECD). In some embodiments, binding of the antibody to the extracellular domain of human Siglec-6 when expressed on a surface of a human mast cell induces cleaving of Siglec-6 (e.g., the Siglec-6 ECD).

In some embodiments, an anti-Siglec-6 antibody described herein depletes mast cells expressing human Siglec-6 in vitro and/or in vivo, e.g., in the presence of effector cells. In some embodiments, binding of the antibody to the extracellular domain of human Siglec-6 when expressed on a surface of a human mast cell induces ADCC and/or ADCP activity, e.g., in the presence of effector cells in vitro and/or in vivo. In some other aspects, an anti-Siglec-6 antibody described herein kills mast cells expressing Siglec-6 by ADCC activity in vitro and/or in vivo. In some other aspects, an anti-Siglec-6 antibody described herein induces phagocytosis of mast cells expressing Siglec-6 by ADCP activity in vitro and/or in vivo. In some embodiments, a composition comprises non-fucosylated (i.e., afucosylated) anti-Siglec-6 antibodies. In some embodiments, a composition comprising non-fucosylated anti-Siglec-6 antibodies described herein enhances ADCC activity as compared to a composition comprising partially fucosylated anti-Siglec-6 antibodies.

Assays for assessing ADCC activity are well known in the art and described herein. In an exemplary assay, to measure ADCC activity, effector cells and target cells are used, e.g., at a specific ratio (e.g., 1:50 target cells:effector cells) in the presence of an antibody to be evaluated. Examples of effector cells include natural killer (NK) cells, large granular lymphocytes (LGL), lymphokine-activated killer (LAK) cells and PBMC comprising NK and LGL, or leukocytes having Fc receptors on the cell surfaces, such as neutrophils, eosinophils and macrophages. The target cell is any cell which expresses on the cell surface antigens that antibodies to be evaluated can recognize. An example of such a target cell is a mast cell which expresses Siglec-6 on the cell surface. Target cells are labeled with a reagent that enables detection of cytolysis. Examples of reagents for labeling include a radio-active substance such as sodium chromate (Na₂⁵¹CrO₄) and carboxyfluorescein succinimidyl ester. See, e.g., Immunology, 14, 181 (1968); J. Immunol. Methods, 172, 227 (1994); and J. Immunol. Methods, 184, 29 (1995). The number of remaining target cells can then be assessed (e.g., by flow cytometry) after co-incubation and normalized (e.g., to number of remaining target cells co-incubated with effector cells treated with an antibody that does not bind to Siglec-6 or treated with no antibody).

Assays for assessing ADCP activity are well known in the art and described herein. For example, target cells can be co-cultured with macrophages (e.g., human monocyte-derived macrophages), monocytes, or PBMCs at a specific ratio (e.g., 1:50 target cells:macrophages) in the presence of an antibody to be evaluated. The number of remaining target cells can then be assessed (e.g., by flow cytometry) after co-incubation and normalized (e.g., to number of remaining target cells co-incubated with macrophages treated with an antibody that does not bind to Siglec-6, or treated with no antibody).

In one aspect, an anti-Siglec-6 antibody described herein is a monoclonal antibody. In one aspect, an anti-Siglec-6 antibody described herein is an antibody fragment (including antigen-binding fragment), e.g., a Fab, Fab′-SH, Fv, scFv, or (Fab′)₂ fragment. In one aspect, an anti-Siglec-6 antibody described herein is a chimeric, humanized, or human antibody. In one aspect, any of the anti-Siglec-6 antibodies described herein are purified.

An anti-Siglec-6 antibody described herein may comprise any suitable framework variable domain sequence, provided that the antibody retains the ability to bind human Siglec-6. As used herein, heavy chain framework regions are designated “HC-FR1-FR4,” and light chain framework regions are designated “LC-FR1-FR4.”

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof.

In any of the embodiments herein, the antibody may comprise a heavy chain Fc region, e.g., a human Fc region or human IgG Fc region. In further embodiments, the human IgG Fc region comprises a human IgG1 or IgG4 Fc region. In some embodiments, the human IgG4 Fc region comprises the amino acid substitution S228P, wherein the amino acid residues are numbered according to the EU index as in Kabat. In some embodiments, the human Fc region comprises one or more mutation(s) that reduce effector function.

In some embodiments, the human IgG1 Fc region comprises one or more mutation(s) that reduce effector function. In some embodiments, the human IgG1 Fc region comprises a substitution or deletion at one or more of the following position(s), numbering based on EU index: (a) L234 and/or L235; (b) A327, A330, and/or P331; (c) E233, L234, L235, and/or G236; (d) E233, L234, and/or L235; (e) E233, L234, L235, G236, A327, A330, and/or P331; (f) E233, L234, L235, A327, A330, and/or P331; (g) N297; (h) L242, N297, and/or K334; (i) A287, N297, and/or L306; (j) R292, N297, and/or V302; (k) N297, V323, and/or I332; (l) V259, N297, and/or L306; (m) L234, L235, K322, M252, S254, and/or T256; or (n) L234, L235, and/or P329. In some embodiments, the antibody comprises a human IgG1 Fe region with one or more of the following mutation(s), numbering based on EU index: (a) L234A and/or L235A; (b) A327G, A330S, and/or P331S; (c) E233P, L234V, L235A, and/or G236del; (d) E233P, L234V, and/or L235A; (e) E233P, L234V, L235A, G236del, A327G, A330S, and/or P331S; (f) E233P, L234V, L235A, A327G, A330S, and/or P331S; (g) N297A; (h) N297G; (i) N297Q; (j) L242C, N297C, and/or K334C; (k) A287C, N297G, and/or L306C; (l) R292C, N297G, and/or V302C; (m) N297G, V323C, and/or I332C; (n) V259C, N297G, and/or L306C; (o) L234F, L235Q, K322Q, M252Y, S254T, and/or T256E; (p) L234A, L235A, and/or P329G; or (q) L234A, L235Q, and K322Q. See, e.g., Schlothauer, T. et al. (2016) Protein Eng. Des. Sel. 29:457-466; Armour, K. L. et al. (2003) Mol. Immunol. 40:585-593; Jacobsen, F. W. et al. (2017) J. Biol. Chem. 292:1865-1875; and Borrok, M. J. et al. (2017) J. Pharm. Sci. 106:1008-1017. In some embodiments, the antibody heavy chain comprises a heavy chain constant region comprising the amino acid sequence of SEQ ID NO:101 or 102.

In some embodiments, the anti-Siglec-6 antibody comprises an Fc region that has (i.e., is capable of inducing) one or more effector functions (e.g., in the presence of appropriate effector cell(s)). In some embodiments, the Fc region is an “active” Fc region such as a human IgG1 Fc region (e.g., a wild-type human IgG1 Fc region). In some embodiments, the Fc region has one or more effector functions including but not limited to C1q binding and complement dependent cytotoxicity; Fc receptor binding; antibody-dependent cell-mediated cytotoxicity (ADCC); phagocytosis (e.g., antibody-dependent cell-mediated phagocytosis or ADCP); down regulation of cell surface receptors (e.g., B cell receptors); and B cell activation. In some embodiments, the antibody comprises an Fc region capable of inducing ADCC and/or ADCP activity, e.g., in the presence of one or more effector cells (such as those expressing an appropriate Fc receptor that binds the antibody Fc region).

In some embodiments, the human IgG1 Fc region comprises one or more mutation(s) that increase or enhance effector function. In some embodiments, the human IgG1 Fc region comprises a substitution or deletion at one or more of the following position(s), numbering based on EU index: (a) F243, R292, Y300, V305, and/or P396; (b) S239 and/or I332; (c) S239, I332, and/or A330; (d) S298, E333, and/or K334; (e) G236, S239, and/or I332; (f) K326 and/or E333; (g) S267, H268, and/or S324; or (h) E345, E430, and/or S440. In some embodiments, the human IgG1 Fe region comprises one or more of the following mutation(s), numbering based on EU index: (a) F243L, R292P, Y300L, V305I, and/or P396L; (b) S239D and/or I332E; (c) S239D, I332E, and/or A330L; (d) S298A, E333A, and/or K334A; (e) G236A, S239D, and/or I332E; (f) K326W and/or E333S; (g) S267E, H268F, and/or S324T; or (h) E345R, E430G, and/or S440Y. See, e.g., Stavenhagen, J. B. et al. (2007) Cancer Res. 67:8882-8890; Lazar, G. A. et al. (2006) Proc. Natl. Acad. Sci. USA 103:4005-4010; Shields, R. L. et al. (2001) J. Biol. Chem. 276:6591-6604; Richards, J. O. et al. (2008) Mol. Cancer Ther. 7:2517-2527; Idusogie, E. E. et al. (2001) J. Immunol. 166:2571-2575; Moore, G. L. et al. (2010) MAbs 2:181-189; and Diebolder, C. A. et al. (2014) Science 343:1260-1263.

In some embodiments, the human IgG2 Fe region comprises one or more mutation(s) that reduce effector function. In some embodiments, the human IgG2 Fc region comprises a substitution or deletion at one or more of the following position(s), numbering based on EU index: (a) A330 and/or P331; (b) V234, G237, P238, H268, V309, A330, and/or P331; or (c) V234, G237, H268, V309, A330, P331, C232, C233, S267, L328, M252, S254, and/or T256. In some embodiments, the human IgG2 Fc region comprises one or more of the following mutation(s), numbering based on EU index: (a) A330S and/or P331S; (b) V234A, G237A, P238S, H268A, V309L, A330S, and/or P331S; or (c) V234A, G237A, H268Q, V309L, A330S, P331S, C232S, C233S, S267E, L328F, M252Y, S254T, and/or T256E. See, e.g., Armour, K. L. et al. (2003) Mol. Immunol. 40:585-593 and US PG Pub. Nos. 20170204193 and 20170240631.

In some embodiments, the human IgG4 Fc region comprises one or more mutation(s) that reduce effector function. In some embodiments, the human IgG4 Fc region comprises a substitution or deletion at one or more of the following position(s), numbering based on EU index: (a) E233, F234, L235, and/or G236; (b) E233, F234, and/or L235; or (c) S228 and/or L235. In some embodiments, the human IgG4 Fc region comprises one or more of the following mutation(s), numbering based on EU index: (a) E233P, F234V, L235A, and/or G236del; (b) E233P, F234V, and/or L235A; (c) S228P and/or L235E; or (d) S228P and/or L235A. See, e.g., Schlothauer, T. et al. (2016) Protein Eng. Des. Sel. 29:457-466; and Armour, K. L. et al. (2003) Mol. Immunol. 40:585-593. In some embodiments, the antibody heavy chain comprises a heavy chain constant region comprising the amino acid sequence of SEQ ID NO:103.

In some embodiments, the anti-Siglec-6 antibody of the present disclosure comprises an antibody heavy chain comprising a heavy chain constant region that comprises an amino acid sequence shown in Table 3.

TABLE 3
Antibody heavy chain constant region sequences.

| SEQ ID | Sequence | |
|---|---|---|
| NO | Description | Sequence |
| 101 | Human IgG1 | ASTKGPSVFPLAPSSKSTSGGTAALGCLVKDYFPEPVTVSWNSG |
| | constant region | ALTSGVHTFPAVLQSSGLYSLSSVVTVPSSSLGTQTYICNVNHKP |
| | (f allotype) | SNTKVDKRVEPKSCDKTHTCPPCPAPELLGGPSVFLFPPKPKDTL |
| | | MISRTPEVTCVVVDVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGNVF |
| | | SCSVMHEALHNHYTQKSLSLSPG |
| 102 | Human IgG1 | ASTKGPSVFPLAPSSKSTSGGTAALGCLVKDYFPEPVTVSWNSG |
| | constant region | ALTSGVHTFPAVLQSSGLYSLSSVVTVPSSSLGTQTYICNVNHKP |
| | (z allotype) | SNTKVDKKVEPKSCDKTHTCPPCPAPELLGGPSVFLFPPKPKDTL |
| | | MISRTPEVTCVVVDVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGNVF |
| | | SCSVMHEALHNHYTQKSLSLSPG |
| 103 | Human IgG4 | ASTKGPSVFPLAPCSRSTSESTAALGCLVKDYFPEPVTVSWNSG |
| | S228P constant | ALTSGVHTFPAVLQSSGLYSLSSVVTVPSSSLGTKTYTCNVDHK |
| | region | PSNTKVDKRVESKYGPPCPPCPAPEFLGGPSVFLFPPKPKDTLMI |
| | | SRTPEVTCVVVDVSQEDPEVQFNWYVDGVEVHNAKTKPREEQF |
| | | NSTYRVVSVLTVLHQDWLNGKEYKCKVSNKGLPSSIEKTISKAK |
| | | GQPREPQVYTLPPSQEEMTKNQVSLTCLVKGFYPSDIAVEWESN |
| | | GQPENNYKTTPPVLDSDGSFFLYSRLTVDKSRWQEGNVFSCSV |
| | | MHEALHNHYTQKSLSLSLG |

In some embodiments, an anti-Siglec-6 antibody of the present disclosure comprises an antibody light chain comprising a light chain constant (CL) domain, e.g., a human kappa or lambda CL domain. In some embodiments, the CL domain is a human kappa CL domain. In some embodiments, the CL domain comprises the amino acid sequence of

(SEQ ID NO: 104)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

In some embodiments, an anti-Siglec-6 antibody of the present disclosure comprises a heavy chain comprising the amino acid sequence of QVQLQESGPGLVKPSETLSLTCTVSGFSLTSYGVSWIRQPPGKGLEWIGVIWHDGSTSYH PSLKSRVTISRDTSKNQVSLKLSSVTAADTAVYYCASDGYSGTFAYWGQGTLVTVSSAS TKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKRVEPKSCDKTHTCPPCPAPELLGGP SVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVYTLPPSR EEMTKNQVSLTCLVKGFYPSDIAVEWESNGQPENNYKTTPPVLDSDGSFFLYSKLTVDK SRWQQGNVFSCSVMHEALHNHYTQKSLSLSPG (SEQ ID NO:165) or QVQLQESGPGLVKPSETLSLTCTVSGFSLTSYGVSWIRQPPGKGLEWIGVIWHDGSTSYH PSLKSRVTISRDTSKNQVSLKLSSVTAADTAVYYCASDGYSGTFAYWGQGTLVTVSSAS TKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKRVEPKSCDKTHTCPPCPAPELLGGP SVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVYTLPPSR EEMTKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO:166) and/or a light chain comprising the amino acid sequence of QIVVTQSPATLSLSPGERATLSCTASSSVSSSYLHWYQQKPGQAPRLLIYSTSILASGIPAR FSGSGSGTDFTLTISSLQPEDFAVYYCHQYHRSPYTFGQGTKLEIKRTVAAPSVFIFPPSD EQLKSGTASVVCLLNNFYPREAKVQWKVDNALQSGNSQESVTEQDSKDSTYSLSSTLT LSKADYEKHKVYACEVTHQGLSSPVTKSFNRGEC (SEQ ID NO:167). In some embodiments, the antibody comprises a heavy chain comprising the amino acid sequence of SEQ ID NO:165 or 166 and a light chain comprising the amino acid sequence of SEQ ID NO:167. In some embodiments, one or both of the antibody Fc regions or heavy chains do not have a C-terminal lysine. As is known in the art, the C-terminal lysine of some antibody heavy chain species may be cleaved off in some fraction of molecules. In some embodiments, an anti-Siglec-6 antibody of the present disclosure comprises a heavy chain that comprises the amino acid sequence of SEQ ID NO:165 or 166 and a light chain that comprises the amino acid sequence of SEQ ID NO:167. In some embodiments, an anti-Siglec-6 antibody of the present disclosure comprises a heavy chain that comprises the amino acid sequence of SEQ ID NO:165 and a light chain that comprises the amino acid sequence of SEQ ID NO:167. In some embodiments, an anti-Siglec-6 antibody of the present disclosure comprises a heavy chain that comprises the amino acid sequence of SEQ ID NO:166 and a light chain that comprises the amino acid sequence of SEQ ID NO:167. In some embodiments, a composition of the present disclosure comprises a mixture of anti-Siglec-6 antibody species, wherein each species comprises a heavy chain that comprises the amino acid sequence of SEQ ID NO:165 or 166 and a light chain that comprises the amino acid sequence of SEQ ID NO:167.

In one aspect, the present disclosure provides anti-Siglec-6 antibodies with reduced or eliminated fucosylation, e.g., as described infra. For example, in some embodiments, at least one or two of the heavy chains of the antibody is non-fucosylated. In some embodiments, the Fc region is non-fucosylated. In some embodiments, the antibody comprises a non-fucosylated human IgG1 Fc region. Exemplary assays for measuring antibody fucosylation, as well as methods and cell lines for producing antibodies with altered, reduced, or eliminated fucosylation, are provided herein.

In one aspect, polynucleotides encoding anti-Siglec-6 antibodies are provided. In certain embodiments, vectors comprising polynucleotides encoding anti-Siglec-6 antibodies are provided. In certain embodiments, host cells comprising such vectors are provided. In another aspect of the invention, compositions comprising anti-Siglec-6 antibodies or polynucleotides encoding anti-Siglec-6 antibodies are provided. In certain embodiments, a composition of the present disclosure is a pharmaceutical formulation for the treatment of a mast cell-mediated disorder, such as those enumerated herein.

1. Antibody Affinity

In some embodiments, the anti-Siglec-6 antibody binds to the ECD of human Siglec-6 with an equilibrium dissociation constant (K_{D}) of about 250 pM or less, about 225 pM or less, about 200 pM or less, about 175 pM or less, about 150 pM or less, about 125 pM or less, about 100 pM or less, about 90 pM or less, about 80 pM or less, about 70 pM or less, about 60 pM or less, about 50 pM or less, about 40 pM or less, about 30 pM or less, about 20 pM or less, about 10 pM or less, or about 1 pM. In some embodiments, the anti-Siglec-6 antibody binds to Domain 1 of the ECD of human Siglec-6 with an equilibrium dissociation constant (K_{D}) of about 250 pM or less, about 225 pM or less, about 200 pM or less, about 175 pM or less, about 150 pM or less, about 125 pM or less, about 100 pM or less, about 90 pM or less, about 80 pM or less, about 70 pM or less, about 60 pM or less, about 50 pM or less, about 40 pM or less, about 30 pM or less, about 20 pM or less, about 10 pM or less, or about 1 pM. In some embodiments, the anti-Siglec-6 antibody binds to Domain 2 of the ECD of human Siglec-6 with an equilibrium dissociation constant (K_{D}) of about 250 pM or less, about 225 pM or less, about 200 pM or less, about 175 pM or less, about 150 pM or less, about 125 pM or less, about 100 pM or less, about 90 pM or less, about 80 pM or less, about 70 pM or less, about 60 pM or less, about 50 pM or less, about 40 pM or less, about 30 pM or less, about 20 pM or less, about 10 pM or less, or about 1 pM. In some embodiments, the anti-Siglec-6 antibody binds to Domain 3 of the ECD of human Siglec-6 with an equilibrium dissociation constant (K_{D}) of about 250 pM or less, about 225 pM or less, about 200 pM or less, about 175 pM or less, about 150 pM or less, about 125 pM or less, about 100 pM or less, about 90 pM or less, about 80 pM or less, about 70 pM or less, about 60 pM or less, about 50 pM or less, about 40 pM or less, about 30 pM or less, about 20 pM or less, about 10 pM or less, or about 1 pM.

Exemplary assays for determining binding affinity of an antibody for human Siglec-6, its ECD, or a sub-domain thereof are known in the art and exemplified herein. In one embodiment, the binding affinity of the anti-Siglec-6 antibody can be determined by a surface plasmon resonance assay. For example, the Kd or Kd value can be measured by using a BIAcore™-2000 or a BIAcore™-3000 (BIAcore, Inc., Piscataway, N.J.) at 25° C. with immobilized antigen CM5 chips at ˜10 response units (RU). Briefly, carboxymethylated dextran biosensor chips (CM5, BIAcore® Inc.) are activated with N-ethyl-N′-(3-dimethylaminopropyl)-carbodiimide hydrochloride (EDC) and N-hydroxysuccinimide (NHS) according to the supplier's instructions. Capture antibodies (e.g., anti-human-Fc) are diluted with 10 mM sodium acetate, pH 4.8, before injection at a flow rate of 30 μl/minute and further immobilized with an anti-Siglec-6 antibody. For kinetics measurements, two-fold serial dilutions of dimeric Siglec-6 are injected in PBS with 0.05% Tween 20 (PBST) at 25° C. at a flow rate of approximately 25 μl/min. Association rates (kon) and dissociation rates (koff) are calculated using a simple one-to-one Langmuir binding model (BIAcore® Evaluation Software version 3.2) by simultaneously fitting the association and dissociation sensorgrams. The equilibrium dissociation constant (Kd) is calculated as the ratio koff/kon. See, e.g., Chen, Y., et al., (1999) J. Mol. Biol. 293:865-881.

In another embodiment, biolayer interferometry may be used to determine the affinity of anti-Siglec-6 antibodies against Siglec-6. In an exemplary assay, Siglec-6-Fc tagged protein is immobilized onto anti-human capture sensors, and incubated with increasing concentrations of mouse, chimeric, or humanized anti-Siglec-6 Fab fragments to obtain affinity measurements using an instrument such as, for example, the Octet Red 384 System (ForteBio).

The binding affinity of the anti-Siglec-6 antibody can, for example, also be determined by the Scatchard analysis described in Munson et al., Anal. Biochem., 107:220 (1980) using standard techniques well known in the relevant art. See also Scatchard, G., Ann. N.Y. Acad. Sci. 51:660 (1947).

2. Competition Assays

Competition assays can be used to determine whether two antibodies bind the same epitope by recognizing identical or sterically overlapping epitopes or one antibody competitively inhibits binding of another antibody to the antigen. These assays are known in the art. Typically, antigen or antigen expressing cells is immobilized on a multi-well plate and the ability of unlabeled antibodies to block the binding of labeled antibodies is measured. Common labels for such competition assays are radioactive labels or enzyme labels. In some embodiments, an anti-Siglec-6 antibody described herein competes with a reference antibody described herein for binding to a Siglec-6 polypeptide or an ECD or domain thereof, e.g., expressed on the cell surface of a cell (e.g., a mast cell).

### III. Antibody Preparation

The antibody described herein is prepared using techniques available in the art for generating antibodies, exemplary methods of which are described in more detail in the following sections.

1. Antibody Fragments

The present invention encompasses antibody fragments. Antibody fragments may be generated by traditional means, such as enzymatic digestion, or by recombinant techniques. In certain circumstances there are advantages of using antibody fragments, rather than whole antibodies. For a review of certain antibody fragments, see Hudson et al. (2003) Nat. Med. 9:129-134.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. Fab and F(ab′)₂ fragment with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870, for example. Such linear antibodies may be monospecific or bispecific.

2. Humanized Antibodies

The present disclosure encompasses humanized antibodies. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter (Jones et al. (1986) Nature 321:522-525; Riechmann et al. (1988) Nature 332:323-327; Verhoeyen et al. (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies can be important to reduce antigenicity. According to the so-called “best-fit” method, the sequence of the variable domain of a rodent (e.g., mouse) antibody is screened against the entire library of known human variable-domain sequences. The human sequence which is closest to that of the rodent is then accepted as the human framework for the humanized antibody (Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623.

It is further generally desirable that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those, skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

3. Human Antibodies

Human anti-Siglec-6 antibodies of the invention can be constructed by combining Fv clone variable domain sequence(s) selected from human-derived phage display libraries with known human constant domain sequences(s). Alternatively, human monoclonal anti-Siglec-6 antibodies of the invention can be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).

It is possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90: 2551 (1993); Jakobovits et al., Nature, 362: 255 (1993); Bruggermann et al., Year in Immunol., 7: 33 (1993).

Gene shuffling can also be used to derive human antibodies from non-human (e.g., rodent) antibodies, where the human antibody has similar affinities and specificities to the starting non-human antibody. According to this method, which is also called “epitope imprinting”, either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e., the epitope governs the choice of the human chain partner. When the process is repeated in order to replace the remaining non-human chain, a human antibody is obtained (see PCT WO 93/06213 published Apr. 1, 1993). Unlike traditional humanization of non-human antibodies by CDR grafting, this technique provides completely human antibodies, which have no FR or CDR residues of non-human origin.

4. Multispecific Antibodies

Multispecific antibodies are monoclonal antibodies that have binding specificities for at least two different antigens. Bispecific antibodies may refer to antibodies that have binding specificities for two different antigens, or two different epitopes on the same antigen. In certain embodiments, bispecific antibodies are human or humanized antibodies. In certain embodiments, one of the binding specificities is for Siglec-6 and the other is for any other antigen. In certain embodiments, bispecific antibodies may bind to two different epitopes of Siglec-6. Bispecific antibodies may also be used to localize cytotoxic agents to cells which express Siglec-6. Bispecific antibodies can be prepared as full length antibodies or antibody fragments (e.g. F(ab′)₂ bispecific antibodies).

Methods for making bispecific antibodies are known in the art. See Milstein and Cuello, Nature, 305: 537 (1983), WO 93/08829 published May 13, 1993, and Traunecker et al., EMBO J., 10: 3655 (1991). For further details of generating bispecific antibodies see, for example, Suresh et al., Methods in Enzymology, 121:210 (1986). Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking method. Suitable cross-linking agents are well known in the art, and are disclosed in U.S. Pat. No. 4,676,980, along with a number of cross-linking techniques.

5. Single-Domain Antibodies

In some embodiments, an antibody of the invention is a single-domain antibody. A single-domain antibody is a single polypeptide chain comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1). In one embodiment, a single-domain antibody consists of all or a portion of the heavy chain variable domain of an antibody.

6. Antibody Variants

In some embodiments, amino acid sequence modification(s) of the antibodies described herein are contemplated. For example, it may be desirable to improve the binding affinity and/or other biological properties of the antibody. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085. Here, a residue or group of target residues are identified (e.g., charged residues such as arg, asp, his, lys, and glu) and replaced by a neutral or negatively charged amino acid (e.g., alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

In certain embodiments, an antibody of the invention is altered to increase or decrease the extent to which the antibody is glycosylated. Glycosylation of polypeptides is typically either N-linked or O-linked. N-linked refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition or deletion of glycosylation sites to the antibody is conveniently accomplished by altering the amino acid sequence such that one or more of the above-described tripeptide sequences (for N-linked glycosylation sites) is created or removed. The alteration may also be made by the addition, deletion, or substitution of one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US Pat Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fe region thereof. See also US 2005/0123546 (Umana et al.) on antigen-binding molecules with modified glycosylation.

In certain embodiments, a glycosylation variant comprises an Fc region, wherein a carbohydrate structure attached to the Fc region lacks fucose or has reduced fucose. Such variants have improved ADCC function. Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)), and cells overexpressing β1,4-N-acetylglucosaminyltransferase III (GnT-III) and Golgi μ-mannosidase II (ManII).

Antibodies are contemplated herein that have reduced fucose relative to the amount of fucose on the same antibody produced in a wild-type CHO cell. For example, the antibody has a lower amount of fucose than it would otherwise have if produced by native CHO cells (e.g., a CHO cell that produce a native glycosylation pattern, such as, a CHO cell containing a native FUT8 gene). In certain embodiments, an anti-Siglec-6 antibody provided herein is one wherein less than about 50%, 40%, 30%, 20%, 10%, 5% or 1% of the N-linked glycans thereon comprise fucose. In certain embodiments, an anti-Siglec-6 antibody provided herein is one wherein none of the N-linked glycans thereon comprise fucose, i.e., wherein the antibody is completely without fucose, or has no fucose or is non-fucosylated or is afucosylated. The amount of fucose can be determined by calculating the average amount of fucose within the sugar chain at Asn297, relative to the sum of all glycostructures attached to Asn297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. In some embodiments, at least one or two of the heavy chains of the antibody is non-fucosylated.

In one embodiment, the antibody is altered to improve its serum half-life. To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule (US 2003/0190311, U.S. Pat. Nos. 6,821,505; 6,165,745; 5,624,821; 5,648,260; 6,165,745; 5,834,597).

Another type of variant is an amino acid substitution variant. These variants have at least one amino acid residue in the antibody molecule replaced by a different residue. Sites of interest for substitutional mutagenesis include the hypervariable regions, but FR alterations are also contemplated. Conservative substitutions are shown in Table 1 under the heading of “preferred substitutions.” If such substitutions result in a desirable change in biological activity, then more substantial changes, denominated “exemplary substitutions” in Table 1, or as further described below in reference to amino acid classes, may be introduced and the products screened.

TABLE 1

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; | Ile |
| | Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or c) the bulk of the side chain. Amino acids may be grouped according to similarities in the properties of their side chains (in A. L. Lehninger, in Biochemistry, second ed., pp. 73-75, Worth Publishers, New York (1975)):
(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gin (Q)
(3) acidic: Asp (D), Glu (E)
(4) basic: Lys (K), Arg (R), His (H)

Alternatively, naturally occurring residues may be divided into groups based on common side-chain properties:
(1) hydrophobic: Norleucine, Met, Ala, Val, Leu, lie;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class. Such substituted residues also may be introduced into the conservative substitution sites or, into the remaining (non-conserved) sites.

One type of substitutional variant involves substituting one or more hypervariable region residues of a parent antibody (e.g., a humanized or human antibody). Generally, the resulting variant(s) selected for further development will have modified (e.g., improved) biological properties relative to the parent antibody from which they are generated. A convenient way for generating such substitutional variants involves affinity maturation using phage display. Briefly, several hypervariable region sites (e.g., 6-7 sites) are mutated to generate all possible amino acid substitutions at each site. The antibodies thus generated are displayed from filamentous phage particles as fusions to at least part of a phage coat protein (e.g., the gene III product of M13) packaged within each particle. The phage-displayed variants are then screened for their biological activity (e.g., binding affinity). In order to identify candidate hypervariable region sites for modification, scanning mutagenesis (e.g., alanine scanning) can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to techniques known in the art, including those elaborated herein. Once such variants are generated, the panel of variants is subjected to screening using techniques known in the art, including those described herein, and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to introduce one or more amino acid modifications in an Fc region of antibodies of the present disclosure, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions including that of a hinge cysteine. In some embodiments, the Fc region variant comprises a human IgG1, IgG2, or IgG4 Fc region. Exemplary Fc region variants are provided herein.

In accordance with this description and the teachings of the art, it is contemplated that in some embodiments, an antibody of the invention may comprise one or more alterations as compared to the wild type counterpart antibody, e.g. in the Fc region. These antibodies would nonetheless retain substantially the same characteristics required for therapeutic utility as compared to their wild type counterpart. For example, it is thought that certain alterations can be made in the Fc region that would result in altered (i.e., either improved or diminished) C1q binding and/or Complement Dependent Cytotoxicity (CDC), e.g., as described in WO99/51642. See also Duncan & Winter Nature 322:738-40 (1988); U.S. Pat. Nos. 5,648,260; 5,624,821; and WO94/29351 concerning other examples of Fc region variants. WO00/42072 (Presta) and WO 2004/056312 (Lowman) describe antibody variants with improved or diminished binding to FcRs. The content of these patent publications are specifically incorporated herein by reference. See, also, Shields et al. J. Biol. Chem. 9(2): 6591-6604 (2001). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). These antibodies comprise an Fc region with one or more substitutions therein which improve binding of the Fc region to FcRn. Polypeptide variants with altered Fc region amino acid sequences and increased or decreased C1q binding capability are described in U.S. Pat. No. 6,194,551B1, WO99/51642. The contents of those patent publications are specifically incorporated herein by reference. See, also, Idusogie et al. J. Immunol. 164: 4178-4184 (2000).

7. Vectors, Host Cells, and Recombinant Methods

For recombinant production of an antibody of the invention, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, host cells are of either prokaryotic or eukaryotic (generally mammalian) origin. It will be appreciated that constant regions of any isotype can be used for this purpose, including IgG, IgM, IgA, IgD, and IgE constant regions, and that such constant regions can be obtained from any human or animal species.

Generating Antibodies Using Prokarvotic Host Cells:

a) Vector Construction

Polynucleotide sequences encoding polypeptide components of the antibody of the invention can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes-encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as λGEM™-11 may be utilized in making a recombinant vector which can be used to transform susceptible host cells such as E. coli LE392.

The expression vector of the invention may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the (3-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB-strains) provide cytoplasm conditions that are favorable for disulfide bond formation, thereby permitting proper folding and assembly of expressed protein subunits. Proba and Pluckthun Gene, 159:203 (1995).

Antibodies of the invention can also be produced by using an expression system in which the quantitative ratio of expressed polypeptide components can be modulated in order to maximize the yield of secreted and properly assembled antibodies of the invention. Such modulation is accomplished at least in part by simultaneously modulating translational strengths for the polypeptide components.

One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence. In certain embodiments, changes in the nucleotide sequence are silent. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A Companion to Methods in Enzymol. 4:151-158.

In one embodiment, a set of vectors is generated with a range of TIR strengths for each cistron therein. This limited set provides a comparison of expression levels of each chain as well as the yield of the desired antibody products under various TIR strength combinations. TIR strengths can be determined by quantifying the expression level of a reporter gene as described in detail in Simmons et al. U.S. Pat. No. 5,840,523. Based on the translational strength comparison, the desired individual TIRs are selected to be combined in the expression vector constructs of the invention.

Prokaryotic host cells suitable for expressing antibodies of the invention include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, gram-negative cells are used. In one embodiment, E. coli cells are used as hosts for the invention. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 ΔfhuA (ΔtonA) ptr3 lac Iq lacL8 ΔompTΔ(nmpc-fepE) degP41 kanR (U.S. Pat. No. 5,639,635). Other strains and derivatives thereof, such as E. coli 294 (ATCC 31,446), E. coli B, E. coli Δ 1776 (ATCC 31,537) and E. coli RV308(ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

b) Antibody Production

Host cells are transformed with the above-described expression vectors and cultured in conventional nutrient media modified as appropriate for inducing promoters, selecting transformants, or amplifying the genes encoding the desired sequences.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the invention are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. In certain embodiments, for E. coli growth, growth temperatures range from about 20° C. to about 39° C.; from about 25° C. to about 37° C.; or about 30° C. The pH of the medium may be any pH ranging from about 5 to about 9, depending mainly on the host organism. In certain embodiments, for E. coli, the pH is from about 6.8 to about 7.4, or about 7.0.

If an inducible promoter is used in the expression vector of the invention, protein expression is induced under conditions suitable for the activation of the promoter. In one aspect of the invention, PhoA promoters are used for controlling transcription of the polypeptides. Accordingly, the transformed host cells are cultured in a phosphate-limiting medium for induction. In certain embodiments, the phosphate-limiting medium is the C.R.A.P. medium (see, e.g., Simmons et al., J. Immunol. Methods (2002), 263:133-147). A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In one aspect of the invention, antibody production is conducted in large quantity by a fermentation process. Various large-scale fed-batch fermentation procedures are available for production of recombinant proteins. Large-scale fermentations have at least 1000 liters of capacity, and in certain embodiments, about 1,000 to 100,000 liters of capacity. These fermentors use agitator impellers to distribute oxygen and nutrients, especially glucose. Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J. Biol. Chem. 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the invention.

c) Antibody Purification

In one embodiment, the antibody protein produced herein is further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In one aspect, Protein A immobilized on a solid phase is used for immunoaffinity purification of the antibody products of the invention. Protein A is a 41 kD cell wall protein from Staphylococcus aureas which binds with a high affinity to the Fc region of antibodies. Lindmark et al (1983) J. Immunol. Meth. 62:1-13. The solid phase to which Protein A is immobilized can be a column comprising a glass or silica surface, or a controlled pore glass column or a silicic acid column. In some applications, the column is coated with a reagent, such as glycerol, to possibly prevent nonspecific adherence of contaminants.

As the first step of purification, a preparation derived from the cell culture as described above can be applied onto a Protein A immobilized solid phase to allow specific binding of the antibody of interest to Protein A. The solid phase would then be washed to remove contaminants non-specifically bound to the solid phase. Finally the antibody of interest is recovered from the solid phase by elution.

Generating Antibodies Using Eukaryotic Host Cells:

A vector for use in a eukaryotic host cell generally includes one or more of the following non-limiting components: a signal sequence, an origin of replication, one or more marker genes, an enhancer element, a promoter, and a transcription termination sequence.

a) Signal Sequence Component

A vector for use in a eukaryotic host cell may also contain a signal sequence or other polypeptide having a specific cleavage site at the N-terminus of the mature protein or polypeptide of interest. The heterologous signal sequence selected may be one that is recognized and processed (i.e., cleaved by a signal peptidase) by the host cell. In mammalian cell expression, mammalian signal sequences as well as viral secretory leaders, for example, the herpes simplex gD signal, are available. The DNA for such a precursor region is ligated in reading frame to DNA encoding the antibody.

b) Origin of Replication

Generally, an origin of replication component is not needed for mammalian expression vectors. For example, the SV40 origin may typically be used only because it contains the early promoter.

c) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, primate metallothionein genes, adenosine deaminase, ornithine decarboxylase, etc.

For example, in some embodiments, cells transformed with the DHFR selection gene are first identified by culturing all of the transformants in a culture medium that contains methotrexate (Mtx), a competitive antagonist of DHFR. In some embodiments, an appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199. Host cells may include NSO, CHOK1, CHOK1SV or derivatives, including cell lines deficient in glutamine synthetase (GS). Methods for the use of GS as a selectable marker for mammalian cells are described in U.S. Pat. Nos. 5,122,464 and 5,891,693.

d) Promoter Component

Expression and cloning vectors usually contain a promoter that is recognized by the host organism and is operably linked to nucleic acid encoding a polypeptide of interest (e.g., an antibody). Promoter sequences are known for eukaryotes. For example, virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. In certain embodiments, any or all of these sequences may be suitably inserted into eukaryotic expression vectors.

Transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982), describing expression of human β-interferon cDNA in mouse cells under the control of a thymidine kinase promoter from herpes simplex virus. Alternatively, the Rous Sarcoma Virus long terminal repeat can be used as the promoter.

e) Enhancer Element Component

Transcription of DNA encoding an antibody of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the human cytomegalovirus early promoter enhancer, the mouse cytomegalovirus early promoter enhancer, the polyoma enhancer on the late side of the replication origin, and adenovirus enhancers. See also Yaniv, Nature 297:17-18 (1982) describing enhancer elements for activation of eukaryotic promoters. The enhancer may be spliced into the vector at a position 5′ or 3′ to the antibody polypeptide-encoding sequence, but is generally located at a site 5′ from the promoter.

f) Transcription Termination Component

Expression vectors used in eukaryotic host cells may also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antibody. One useful transcription termination component is the bovine growth hormone polyadenylation region. See WO94/11026 and the expression vector disclosed therein.

g) Selection and Transformation of Host Cells

Suitable host cells for cloning or expressing the DNA in the vectors herein include higher eukaryote cells described herein, including insect or vertebrate host cells. Propagation of insect or vertebrate cells in culture (tissue culture) has become a routine procedure. Examples of useful insect cell lines are Sf-9 and Sf-21 of Spodoptera frugiperda, DS2 cells of Drosophila melanogaster, or High Five cells (BTI-TN-5B1-4) of Trichopulsia ni. See, e.g., Frenzel, A. et al. (2013) Front. Immunol. 4:217. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; CHOK1 cells, CHOK1SV cells or derivatives and a human hepatoma line (Hep G2).

Host cells are transformed with the above-described-expression or cloning vectors for antibody production and cultured in conventional nutrient media modified as appropriate for inducing promoters, selecting transformants, or amplifying the genes encoding the desired sequences.

h) Culturing the Host Cells

The host cells used to produce an antibody of this invention may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other supplements may also be included at appropriate concentrations that would be known to those skilled in the art. The culture conditions, such as temperature, pH, and the like, are those previously used with the host cell selected for expression, and will be apparent to the ordinarily skilled artisan.

i) Purification of Antibody

When using recombinant techniques, the antibody can be produced intracellularly, or directly secreted into the medium. If the antibody is produced intracellularly, as a first step, the particulate debris, either host cells or lysed fragments, may be removed, for example, by centrifugation or ultrafiltration. Where the antibody is secreted into the medium, supernatants from such expression systems may be first concentrated using a commercially available protein concentration filter, for example, an Amicon or Millipore Pellicon ultrafiltration unit. A protease inhibitor such as PMSF may be included in any of the foregoing steps to inhibit proteolysis, and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a convenient technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Methods 62:1-13 (1983)). Protein G is recommended for all mouse isotypes and for human γ3 (Guss et al., EMBO J. 5:15671575 (1986)). The matrix to which the affinity ligand is attached may be agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to further purification, for example, by low pH hydrophobic interaction chromatography using an elution buffer at a pH between about 2.5-4.5, performed at low salt concentrations (e.g., from about 0-0.25M salt).

In general, various methodologies for preparing antibodies for use in research, testing, and clinical use are well-established in the art, consistent with the above-described methodologies and/or as deemed appropriate by one skilled in the art for a particular antibody of interest.

Production of Non-Fucosylated Antibodies

Provided herein are methods for preparing antibodies with a reduced degree of fucosylation. For example, methods contemplated herein include, but are not limited to, use of cell lines deficient in protein fucosylation (e.g., Lec13 CHO cells, alpha-1,6-fucosyltransferase gene knockout CHO cells, cells overexpressing β1,4-N-acetylglucosaminyltransferase III and further overexpressing Golgi μ-mannosidase II, etc.), and addition of a fucose analog(s) in a cell culture medium used for the production of the antibodies. See Ripka et al. Arch. Biochem. Biophys. 249:533-545 (1986); US Pat Appl No US 2003/0157108 A1, Presta, L; WO 2004/056312 A1; Yamane-Ohnuki et al. Biotech. Bioeng. 87: 614 (2004); and U.S. Pat. No. 8,574,907. Additional techniques for reducing the fucose content of antibodies include Glymaxx technology described in U.S. Patent Application Publication No. 2012/0214975. Additional techniques for reducing the fucose content of antibodies also include the addition of one or more glycosidase inhibitors in a cell culture medium used for the production of the antibodies. Glycosidase inhibitors include α-glucosidase I, α-glucosidase IL and α-mannosidase I. In some embodiments, the glycosidase inhibitor is an inhibitor of α-mannosidase I (e.g., kifunensine).

As used herein, “core fucosylation” refers to addition of fucose (“fucosylation”) to N-acetylglucosamine (“GlcNAc”) at the reducing terminal of an N-linked glycan. Also provided are antibodies produced by such methods and compositions thereof.

In some embodiments, fucosylation of complex N-glycoside-linked sugar chains bound to the Fc region (or domain) is reduced. As used herein, a “complex N-glycoside-linked sugar chain” is typically bound to asparagine 297 (according to the number of Kabat), although a complex N-glycoside linked sugar chain can also be linked to other asparagine residues. A “complex N-glycoside-linked sugar chain” excludes a high mannose type of sugar chain, in which only mannose is incorporated at the non-reducing terminal of the core structure, but includes 1) a complex type, in which the non-reducing terminal side of the core structure has one or more branches of galactose-N-acetylglucosamine (also referred to as “gal-GlcNAc”) and the non-reducing terminal side of Gal-GlcNAc optionally has a sialic acid, bisecting N-acetylglucosamine or the like; or 2) a hybrid type, in which the non-reducing terminal side of the core structure has both branches of the high mannose N-glycoside-linked sugar chain and complex N-glycoside-linked sugar chain.

In some embodiments, the “complex N-glycoside-linked sugar chain” includes a complex type in which the non-reducing terminal side of the core structure has zero, one or more branches of galactose-N-acetylglucosamine (also referred to as “gal-GlcNAc”) and the non-reducing terminal side of Gal-GlcNAc optionally further has a structure such as a sialic acid, bisecting N-acetylglucosamine or the like.

According to the present methods, typically only a minor amount of fucose is incorporated into the complex N-glycoside-linked sugar chain(s). For example, in various embodiments, less than about 60%, less than about 50%, less than about 40%, less than about 30%, less than about 20%, less than about 15%, less than about 10%, less than about 5%, or less than about 1% of the antibody has core fucosylation by fucose in a composition. In some embodiments, substantially none (i.e., less than about 0.5%) of the antibody has core fucosylation by fucose in a composition. In some embodiments, more than about 40%, more than about 50%, more than about 60%, more than about 70%, more than about 80%, more than about 90%, more than about 91%, more than about 92%, more than about 93%, more than about 94%, more than about 95%, more than about 96%, more than about 97%, more than about 98%, or more than about 99% of the antibody is nonfucosylated in a composition.

In some embodiments, provided herein is an antibody wherein substantially none (i.e., less than about 0.5%) of the N-glycoside-linked carbohydrate chains contain a fucose residue. In some embodiments, provided herein is an antibody wherein at least one or two of the heavy chains of the antibody is non-fucosylated.

As described above, a variety of mammalian host-expression vector systems can be utilized to express an antibody. In some embodiments, the culture media is not supplemented with fucose. In some embodiments, an effective amount of a fucose analog is added to the culture media. In this context, an “effective amount” refers to an amount of the analog that is sufficient to decrease fucose incorporation into a complex N-glycoside-linked sugar chain of an antibody by at least about 10%, at least about 20%, at least about 30%, at least about 40% or at least about 50%. In some embodiments, antibodies produced by the instant methods comprise at least about 10%, at least about 20%, at least about 30%, at least about 40% or at least about 50% non-core fucosylated protein (e.g., lacking core fucosylation), as compared with antibodies produced from the host cells cultured in the absence of a fucose analog.

The content (e.g., the ratio) of sugar chains in which fucose is not bound to N-acetylglucosamine in the reducing end of the sugar chain versus sugar chains in which fucose is bound to N-acetylglucosamine in the reducing end of the sugar chain can be determined, for example, as described in the Examples. Other methods include hydrazinolysis or enzyme digestion (see, e.g., Biochemical Experimentation Methods 23: Method for Studying Glycoprotein Sugar Chain (Japan Scientific Societies Press), edited by Reiko Takahashi (1989)), fluorescence labeling or radioisotope labeling of the released sugar chain and then separating the labeled sugar chain by chromatography. Also, the compositions of the released sugar chains can be determined by analyzing the chains by the HPAEC-PAD method (see, e.g., J. Liq Chromatogr. 6:1557 (1983)). (See generally U.S. Patent Application Publication No. 2004/0110282.).

### IV. Compositions

In some aspects, also provided herein are compositions (e.g., pharmaceutical composition) comprising any of the anti-Siglec-6 antibodies described herein. In some aspects, provided herein is a composition comprising an anti-Siglec-6 antibody described herein, wherein the antibody comprises a Fc region and N-glycoside-linked carbohydrate chains linked to the Fc region, wherein less than about 50% of the N-glycoside-linked carbohydrate chains contain a fucose residue. In some aspects, provided herein is a composition comprising an anti-Siglec-6 antibody described herein, wherein the antibody comprises a Fc region and N-glycoside-linked carbohydrate chains linked to the Fc region, wherein substantially none of the N-glycoside-linked carbohydrate chains contain a fucose residue.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000). Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between about 0.1% to about 25% by weight or between about 1 to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

### V. Methods of Treatment

Provided herein are methods for treating a disease or condition characterized by increased activity and/or number of mast cells (e.g., mast cells expressing Siglec-6) in a subject. In some embodiments, the methods comprise administering to a subject an effective amount of an anti-Siglec-6 antibody or composition thereof described herein. Exemplary diseases or conditions characterized by increased activity and/or number of mast cells (e.g., mast cells expressing Siglec-6), i.e., mast cell-mediated disorders or conditions, are described infra.

Further provided herein are methods for inhibiting activation of mast cells (e.g., mast cells expressing Siglec-6) in a subject in need thereof. In some embodiments, the methods comprise administering to a subject an effective amount of an anti-Siglec-6 antibody or composition thereof described herein. In some embodiments, the anti-Siglec-6 antibody inhibits activation of mast cells by at least about 20%, about 30%, about 40%, about 50%, about 60%, about 70%, about 80%, about 90% or about 100%, e.g., as compared to activation of mast cells expressing Siglec-6 in a sample obtained from the subject at a baseline level before treatment.

Further provided herein are methods for depleting mast cells (e.g., mast cells expressing Siglec-6) in a subject in need thereof. In some embodiments, the methods comprise administering to a subject an effective amount of an anti-Siglec-6 antibody or composition thereof described herein. In some embodiments, the anti-Siglec-6 antibody depletes at least about 20%, about 30%, about 40%, about 50%, about 60%, about 70%, about 80%, about 90% or about 100% of the mast cells expressing Siglec-6 in a sample obtained from the subject as compared to a baseline level before treatment.

In some embodiments, the depletion or reduction of mast cells is measured by comparing the mast cell population number in a sample (e.g., a tissue sample or a biological fluid sample) from a subject after treatment with the antibody to the mast cell population number in a sample from a subject before treatment with the antibody. In some embodiments, the depletion or reduction of mast cells is measured by comparing the mast cell population number in a sample (e.g., a tissue sample or a biological fluid sample) from a subject after treatment with the antibody to a reference number or sample, e.g., the mast cell population number in a sample from another subject without the antibody treatment, or average mast cell population number in samples from subjects without the antibody treatment. In some embodiments, depletion of reduction of mast cells is due to ADCC and/or ADCP.

In some embodiments, depletion or reduction of mast cells and/or inhibition of mast cell activation is measured by a reduction or prevention of one or more mast cell products, e.g., preformed or newly formed inflammatory mediators produced from mast cells. Exemplary inflammatory mediators include, but are not limited to, proteases (e.g., pan-tryptase, active or beta-tryptase, chymase, CPA3, heparin, etc.), leukotrienes (e.g., leukotriene C4 or B4, platelet activating factor, prostaglandin D2 or E2, etc.), amines (e.g., histamine, serotonin, dopamine, polyamines, etc.), growth factors (e.g., SCF, GM-CSFG-CSF, FGF, EGF, NGF, VEGF, PDGF, etc.), cytokines (e.g., TNF, IL-1b, IL-4, IL-5, IL-6, IL-8, IL-9, IL-10, IL-13, IL-17, IL-18, IL-31, IL-36, etc.), chemokines (e.g., CCL2, CCL3, CCL4, CCL5, CCL11, CCL12, CCL13, CCL24, CCL26, CXCL1, CXCL2, CXCL3, CXCL4, CXCL5, CXCL6, CXCL7, CXCL8, etc.), exosomes, and extracellular traps.

In some embodiments, administration of the antibody or composition results in a decreased level of an inflammatory mediator or mast cell product in a sample obtained from the subject, e.g., as compared to a level of the inflammatory mediator or mast cell product in a reference number or sample. For example, level of an inflammatory mediator or mast cell product in a sample from the individual obtained after treatment with the antibody or composition can be compared to: a level of the inflammatory mediator or mast cell product in a sample obtained from the subject prior to treatment with the antibody or composition, a level of the inflammatory mediator or mast cell product in a sample obtained from a subject not treated with the antibody or composition, average level of the inflammatory mediator or mast cell product in samples obtained from subject(s) not treated with the antibody or composition, or a reference or normal lab value for level of the inflammatory mediator or mast cell product in a corresponding type of sample. Exemplary inflammatory mediators include, but are not limited to, proteases (e.g., pan-tryptase, active or beta-tryptase, chymase, CPA3, heparin, etc.), leukotrienes (e.g., leukotriene C4 or B4, platelet activating factor, prostaglandin D2 or E2, etc.), amines (e.g., histamine, serotonin, dopamine, polyamines, etc.), growth factors (e.g., SCF, GM-CSFG-CSF, FGF, EGF, NGF, VEGF, PDGF, etc.), cytokines (e.g., TNF, IL-1b, IL-4, IL-5, IL-6, IL-8, IL-9, IL-10, IL-13, IL-17, IL-18, IL-31, IL-36, etc.), chemokines (e.g., CCL2, CCL3, CCL4, CCL5, CCL11, CCL12, CCL13, CCL24, CCL26, CXCL1, CXCL2, CXCL3, CXCL4, CXCL5, CXCL6, CXCL7, CXCL8, etc.), exosomes, and extracellular traps. In some embodiments, the inflammatory mediator or mast cell product is one or more of: active tryptase, CCL2, IL-13, IL-8, CCL4, IL-6, histamine, chymase, CPA3, a prostaglandin (e.g., prostaglandin D2 or E2), a leukotriene (e.g., leukotriene C4 or B4), or a mast cell marker (e.g., CD63, CD107a, CD203c, IgE, or MRGPRX2). In some embodiments, the inflammatory mediator or mast cell product is a substance secreted by mast cells, and the sample is a solid sample (e.g., a biopsy sample), or a liquid sample (e.g., a serum, plasma, or urine sample). In some embodiments, the inflammatory mediator or mast cell product is a substance or marker expressed but not secreted by mast cells, and the sample is a solid sample (e.g., a biopsy sample).

For example, in some embodiments, administration of the antibody or composition results in a decreased level of a mast cell marker in a sample (e.g., a biopsy sample, such as a biopsy sample comprising one or more mast cells) obtained from the subject, e.g., as compared to a level of the mast cell marker in a reference number or sample. In some embodiments, level of a mast cell marker in a sample from the individual obtained after treatment with the antibody or composition can be compared to: a level of the mast cell marker in a sample obtained from the subject prior to treatment with the antibody or composition, a level of the mast cell marker in a sample obtained from a subject not treated with the antibody or composition, average level of the mast cell marker in samples obtained from subject(s) not treated with the antibody or composition, or a reference or normal lab value for level of the mast cell marker in a corresponding type of sample. Exemplary mast cell markers are known in the art and include without limitation CD63, CD107a, CD203c, IgE, and MRGPRX2.

In some embodiments, the sample is a tissue sample (e.g., a biopsy sample, a skin sample, a lung sample, a bone marrow sample, a nasal polyposis sample, etc.). In some embodiments, the sample is a biological fluid sample (e.g., a blood sample, a serum sample, a plasma sample, a urine sample, a bronchoalveolar lavage sample, a nasal lavage sample, etc.).

In some embodiments, the subject has a mast cell-mediated disorder or condition. In some embodiments, the subject is at risk of developing the mast cell-mediated disorder or condition. Exemplary mast cell-mediated disorders and conditions are known in the art and described herein. For example, mast cell-mediated disorders or conditions can include, but are not limited to, mastocytosis (e.g., indolent systemic mastocytosis, ISM; or aggressive systemic mastocytosis, ASM), mast cell leukemia, mast cell activation syndrome, gastroparesis, osteoporosis, osteopenia, renal osteodystrophy, bone fracture, Alzheimer's disease, chronic neuropathic pain, hyperalgesia, nonalcoholic fatty liver disease (NAFLD), nonalcoholic steatohepatitis (NASH), graft vs. host disease (GVH), colitis (e.g., microscopic colitis or ulcerative colitis), hereditary alpha tryptasemia, neurofibroma, Kounis syndrome, urticaria (e.g., chronic spontaneous urticaria or an inducible urticaria), atopic dermatitis, contact dermatitis, angioedema, pruigo nodularis, cholangitis, psoriasis, irritable bowel syndrome (IBS), functional dyspepsia, asthma (e.g., eosinophilic or non-eosinophilic asthma), allergy (e.g., food allergy or pseudo allergy), keloid, chronic rhinosinusitis (e.g., with or without nasal polyps), aspirin exacerbated respiratory disease (AERD), chronic obstructive pulmonary disease (COPD), bullous pemphigoid, idiopathic pulmonary fibrosis, systemic sclerosis, interstitital cystitis, hidradenitis suppurativa, alopecia areata, vitiligo, mast cell gastrointestinal disease, Crohn's disease, rheumatoid arthritis, gastroesophageal reflux disease, viral infection, achalasia, postural tachycardia syndrome, amyotrophic lateral sclerosis (ALS), multiple sclerosis (MS), complex regional pain syndrome, or Ehlers-Danlos syndrome. In some embodiments, the subject has or has been diagnosed with mastocytosis (e.g., indolent systemic mastocytosis, ISM; or aggressive systemic mastocytosis, ASM), mast cell leukemia, mast cell activation syndrome, gastroparesis, osteoporosis, osteopenia, renal osteodystrophy, bone fracture, Alzheimer's disease, chronic neuropathic pain, hyperalgesia, nonalcoholic fatty liver disease (NAFLD), nonalcoholic steatohepatitis (NASH), graft vs. host disease (GVH), colitis (e.g., microscopic colitis or ulcerative colitis), hereditary alpha tryptasemia, neurofibroma, Kounis syndrome, urticaria (e.g., chronic spontaneous urticaria or an inducible urticaria), atopic dermatitis, contact dermatitis, angioedema, pruigo nodularis, cholangitis, psoriasis, irritable bowel syndrome (IBS), functional dyspepsia, asthma (e.g., eosinophilic or non-eosinophilic asthma), allergy (e.g., food allergy or pseudo allergy), keloid, chronic rhinosinusitis (e.g., with or without nasal polyps), aspirin exacerbated respiratory disease (AERD), chronic obstructive pulmonary disease (COPD), bullous pemphigoid, idiopathic pulmonary fibrosis, systemic sclerosis, interstitital cystitis, hidradenitis suppurativa, alopecia areata, vitiligo, mast cell gastrointestinal disease, Crohn's disease, rheumatoid arthritis, gastroesophageal reflux disease, viral infection, achalasia, postural tachycardia syndrome, amyotrophic lateral sclerosis (ALS), multiple sclerosis (MS), complex regional pain syndrome, or Ehlers-Danlos syndrome.

In some embodiments, the methods of the present disclosure (e.g., comprising administration of an effective amount of an anti-Siglec-6 antibody or composition thereof described herein) result in a decrease in one or more symptoms in the individual. For example, a level, amount, or presence of one or more symptoms in the individual after treatment can be compared to a level, amount, or presence of one or more symptoms in the individual at a baseline, e.g., prior to treatment. In some embodiments, the one or more symptoms can include without limitation nausea, cramping, constipation, abdominal pain, bloating, vomiting, diarrhea, fatigue, eye pain, light sensitivity, redness, discharge, runny nose, headache, dizziness, brain fog, itching, flushing, sweating, hives, hypotension, shortness of breath, bone pain, joint pain, weight loss, osteoporosis, angioedema, chest pain, anxiety, depression, rapid heartbeat, bronchoconstriction, and general pain.

For the prevention or treatment of disease, the appropriate dosage of an active agent, will depend on the type of disease to be treated, as defined above, the severity and course of the disease, whether the agent is administered for preventive or therapeutic purposes, previous therapy, the subject's clinical history and response to the agent, and the discretion of the attending physician. The agent is suitably administered to the subject at one time or over a series of treatments. In some embodiments of the methods described herein, an interval between administrations of an anti-Siglec-6 antibody described is about one month or longer. In some embodiments, the interval between administrations is about two months, about three months, about four months, about five months, about six months or longer. As used herein, an interval between administrations refers to the time period between one administration of the antibody and the next administration of the antibody. As used herein, an interval of about one month includes four weeks. Accordingly, in some embodiments, the interval between administrations is about four weeks, about eight weeks, about twelve weeks, about sixteen weeks, about twenty weeks, about twenty four weeks, or longer. In some embodiments, the treatment includes multiple administrations of the antibody, wherein the interval between administrations may vary. For example, the interval between the first administration and the second administration is about one month, and the intervals between the subsequent administrations are about three months. In some embodiments, the interval between the first administration and the second administration is about one month, the interval between the second administration and the third administration is about two months, and the intervals between the subsequent administrations are about three months. In some embodiments, an anti-Siglec-6 antibody described herein is administered at a flat dose. In some embodiments, an anti-Siglec-6 antibody described herein is administered to a subject at a dosage from about 150 to about 450 mg per dose. In some embodiments, the anti-Siglec-6 antibody is administered to a subject at a dosage of about any of 150 mg, 200 mg, 250 mg, 300 mg, 350 mg, 400 mg, and 450 mg per dose. In some embodiments, an anti-Siglec-6 antibody described herein is administered at a weight-based dose. In some embodiments, an anti-Siglec-6 antibody described herein is administered to a subject at a dosage from about 0.1 mg/kg to about 10 mg/kg or about 1.0 mg/kg to about 10 mg/kg. In some embodiments, an anti-Siglec-6 antibody described herein is administered to a subject at a dosage of about any of 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, or 10.0 mg/kg. Any of the dosing frequency described above may be used.

### VI. Articles of Manufacture or Kits

In another aspect, an article of manufacture or kit is provided which comprises an anti-Siglec-6 antibody described herein. The article of manufacture or kit may further comprise instructions for use of the antibody in the methods of the present disclosure. Thus, in certain embodiments, the article of manufacture or kit comprises instructions for the use of an anti-Siglec-6 antibody in methods for treating a disease or condition characterized by increased activity and/or number of mast cells (e.g., mast cells expressing Siglec-6), inhibiting activation of mast cells (e.g., mast cells expressing Siglec-6), and/or depleting mast cells (e.g., mast cells expressing Siglec-6), e.g., in a subject in need thereof. In certain embodiments, the individual is a human. In some embodiments, the individual has or is at risk for developing a mast cell-mediated disorder or condition. In some embodiments, the subject has or has been diagnosed with mastocytosis (e.g., indolent systemic mastocytosis, ISM; or aggressive systemic mastocytosis, ASM), mast cell leukemia, mast cell activation syndrome, gastroparesis, osteoporosis, osteopenia, renal osteodystrophy, bone fracture, Alzheimer's disease, chronic neuropathic pain, hyperalgesia, nonalcoholic fatty liver disease (NAFLD), nonalcoholic steatohepatitis (NASH), graft vs. host disease (GVH), colitis (e.g., microscopic colitis or ulcerative colitis), hereditary alpha tryptasemia, neurofibroma, Kounis syndrome, urticaria (e.g., chronic spontaneous urticaria or an inducible urticaria), atopic dermatitis, contact dermatitis, angioedema, pruigo nodularis, cholangitis, psoriasis, irritable bowel syndrome (IBS), functional dyspepsia, asthma (e.g., eosinophilic or non-eosinophilic asthma), allergy (e.g., food allergy or pseudo allergy), keloid, chronic rhinosinusitis (e.g., with or without nasal polyps), aspirin exacerbated respiratory disease (AERD), chronic obstructive pulmonary disease (COPD), bullous pemphigoid, idiopathic pulmonary fibrosis, systemic sclerosis, interstitital cystitis, hidradenitis suppurativa, alopecia areata, vitiligo, mast cell gastrointestinal disease, Crohn's disease, rheumatoid arthritis, gastroesophageal reflux disease, viral infection, achalasia, postural tachycardia syndrome, amyotrophic lateral sclerosis (ALS), multiple sclerosis (MS), complex regional pain syndrome, or Ehlers-Danlos syndrome.

The article of manufacture or kit may further comprise a container. Suitable containers include, for example, bottles, vials (e.g., dual chamber vials), syringes (such as single or dual chamber syringes) and test tubes. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous, or other modes of administration for treating or preventing a mast cell-mediated disorder in an individual. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

In a specific embodiment, the present invention provides kits for a single dose-administration unit. Such kits comprise a container of an aqueous formulation of therapeutic antibody, including both single or multi-chambered pre-filled syringes. Exemplary pre-filled syringes are available from Vetter GmbH, Ravensburg, Germany.

The article of manufacture or kit herein optionally further comprises a container comprising a second medicament, wherein the anti-Siglec-6 antibody is a first medicament, and which article or kit further comprises instructions on the label or package insert for treating the subject with the second medicament, in an effective amount.

In another embodiment, provided herein is an article of manufacture or kit comprising the formulations described herein for administration in an auto-injector device. An auto-injector can be described as an injection device that upon activation, will deliver its contents without additional necessary action from the patient or administrator. They are particularly suited for self-medication of therapeutic formulations when the delivery rate must be constant and the time of delivery is greater than a few moments.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Generation and Characterization of Anti-Siglec-6 Antibodies

Siglec-6 (also known as CD327) is an inhibitory receptor that is selectively expressed on mast cells. Mast cells are considered pathogenic drivers of numerous autoimmune and inflammatory diseases, including but not limited to food allergy, mast cell activation syndrome, mastocytosis, IPF, COPD, and others. See, e.g., Yu, Y. et al. (2018) Front. Immunol. 9:2138; Yokoi, H. et al. (2006) Allergy 61:769-76; and US PG Pub. Nos. US20060269556A1 and US20080267973A1.

This Example describes the generation and characterization of antibodies that bind various epitopes of human Siglec-6 with high affinity and show potent mast cell inhibitory activity in vitro and in vivo.

### Materials and Methods

Generation of Antibodies

Mice were immunized with Siglec-6 ECD-Fc and boosted with Siglec-6 ECD (see, e.g., SEQ ID NO:1). Following immunization, mice with high titer tail bleeds were selected and spleens and lymph nodes were harvested and fused with myeloma cells to generate hybridomas. Supernatants from hydridoma clones were screened against Siglec-6 ECD using ELISA and high affinity clones were selected for variable region sequencing. Variable regions were then cloned into a mouse IgG1 plasmid, recombinantly expressed, and purified for biochemical and functional characterization.

Antibody Characterization

Bivalent binding affinities of IgG for Siglec-6 mAbs were measured by biolayer interferometry using a FortéBio Octet Red 384 instrument using immobilized Siglec-6 ECD Fc protein.

Domain mapping was assessed by ELISA using recombinantly expressed Siglec-6 extracellular domains fused to a human Fc containing the full ECD, domains 1 and 2, or domain 1. Siglec-6 mAbs were tested for binding to each of these domain fusion proteins and assigned domains based on their specific binding properties. Siglec-6 mAbs that bound to all three fusion proteins were assigned domain 1 binders, mAbs that bound to full ECD and domains 1 and 2 were assigned domain 2 binders, and mAbs that bound to only the full ECD protein were assigned domain 3 binders.

Epitope binning was assessed by biolayer interferometry using a FortéBio Octet Red 384 instrument using immobilized Siglec-6 ECD Fc protein. A panel of 16 Siglec-6 mAbs were tested in pairwise fashion by saturating Siglec-6 ECD with one Siglec-6 mAb followed by binding evaluation of a second Siglec-6 mAb. Siglec-6 mAbs were assigned the same bin if binding was reduced or blocked. mAbs were assigned different bins if the no blocking occurred.

Epitope mapping of Siglec-6 mAbs was performed by cross-linking mass spectrometry (XL-MS). XL-MS allows for the direct analysis of non-covalent interactions using high-mass MALDI analysis. Briefly, Siglec-6 mAbs were crosslinked to Siglec-6 ECD using a specially developed cross-linking mixture (Bich, C et al. Anal. Chem., 2010, 82(1), pp 172-179). Following cross-linking, the sample was analyzed using mass spectrometry which characterized the mAb-antigen interacting residues.

Expression of Siglec-6

Fresh human skin, lung, and gastrointestinal tissues were procured from normal donors and digested into single cells using enzymatic and mechanical methods. Following tissue digestion, major immune cells were identified by flow cytometry and stained with an anti-Siglec-6 mAb (R&D systems, MAB2859) to evaluate expression.

Mast Cell Activation Assays

Human mast cells were derived from peripheral blood from healthy donors. Mast cells were stimulated with an anti-FcεRI (250 ng/mL) in the presence of anti-Siglec-6 mAbs or an isotype control (5 μg/mL) for 20 min at 37 C.

Activation marker CD63 was assessed by flow cytometry. Cytokines and chemokines were measured in supernatant using Meso Scale Discovery. Active tryptase was measured by ELISA in cell-free supernatants.

For IL-13 assays, human mast cells were derived from peripheral blood from healthy donors. Mast cells were cultured in the presence of anti-Siglec-6 mAbs or an isotype control (5 μg/mL) overnight at 37 C followed by stimulation with an anti-FcεRI (250 ng/ml). IL-13 was measured by MSD in cell-free supernatants.

Siglec-6 Internalization

Human peripheral blood derived mast cells were cultured with varying concentrations of the Siglec-6 mAbs MAB2859 (see Table 4 below for HVR sequences; R&D Systems Clone #767329), AK04 (see SEQ ID Nos:5-10 for HVR sequences), and AK02 (see SEQ ID Nos:17-22 for HVR sequences) or an isotype control for 18 h at 37 C. Internalization was measured by FACS using a fluorescent-tagged Siglec-6 mAb that binds to a different epitope on Siglec-6.

TABLE 4
MAB2859 HVR sequences.

| SEQ ID | Antibody | Sequence | |
|---|---|---|---|
| NO | Name | Description | Sequence |
| 95 | R&D | HVR-H1 | GYAFTSYWMH |
| 96 | MAB2859 | HVR-H2 | EIYPTNGGTTYNEKFKR |
| 97 | | HVR-H3 | EDFYAMDY |
| 98 | | HVR-L1 | KSSQSLLDSDGKTCLN |
| 99 | | HVR-L2 | LVSKLDS |
| 100 | | HVR-L3 | WQGTHFPYT |

Siglec-6 Mouse Assays

Systemic anaphylaxis was induced in humanized mice containing human mast cells (NSG-SGM3) using the agonist anti-FcεRI mAb (CRA-1). See, e.g., Bryce P J et al. J Allergy Clin Immunol. 2016 September; 138(3):769-779. Humanized mice were dosed with either an isotype control mAb or anti-Siglec-6 mAb (AK04) 24 hours before receiving CRA-1. Systemic anaphylaxis was measured as change in rectal body temperature beginning 10 minutes after CRA-1 administration and evaluating serum levels of mast cell active tryptase, CCL2, IL-6, histamine, and chymase.

### Results

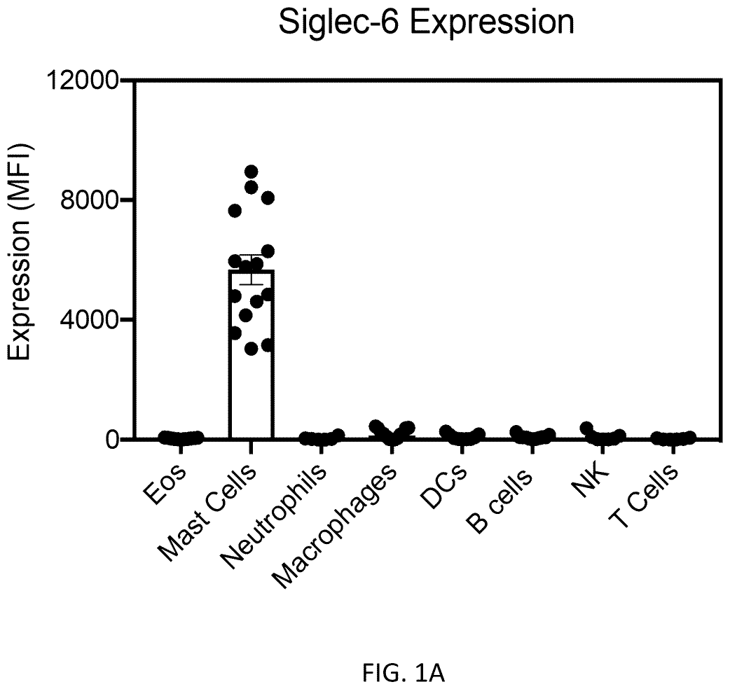

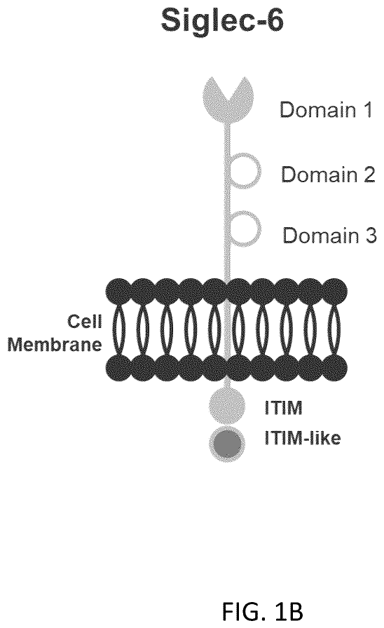

Flow cytometry was used to assess Siglec-6 expression from normal human tissues. Siglec-6 was confirmed to be a marker that is highly and selectively expressed on mast cells (e.g., human primary tissue mast cells), and not other major immune cell types such as eosinophils, neutrophils, macrophages, dendritic cells, B cells, NK cells, or T cells (FIG. 1A).

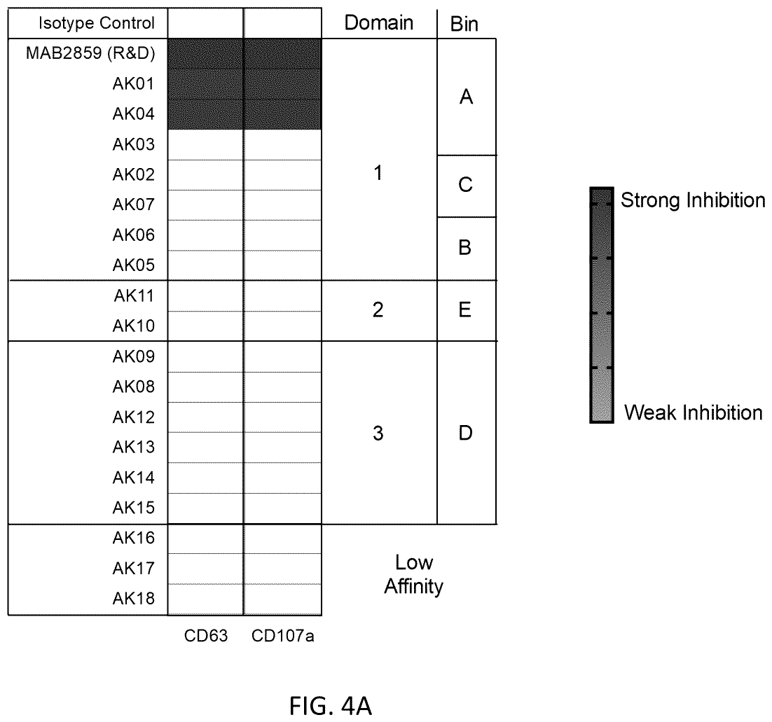

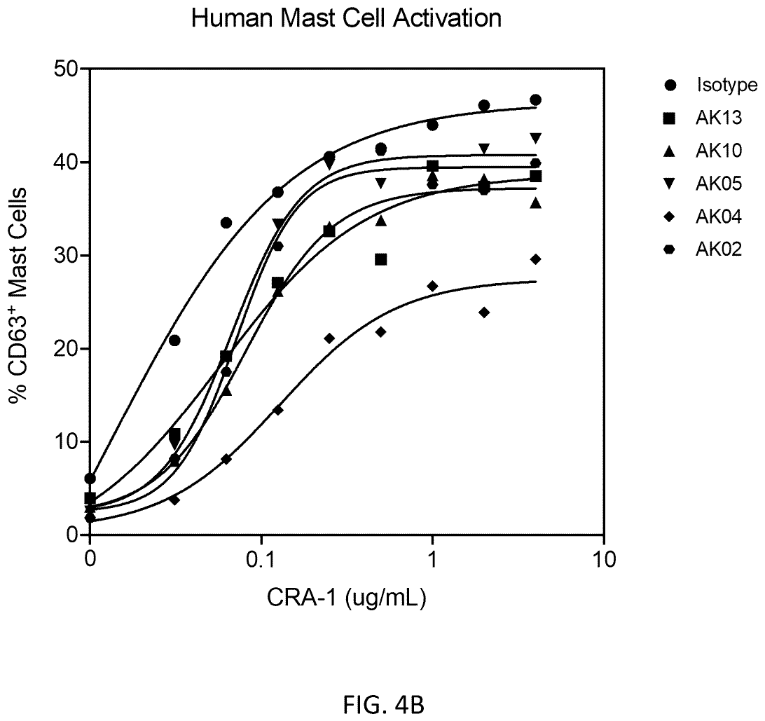

Antibodies that bind to various epitopes across the extracellular domain of human Siglec-6 (FIG. 1B; see also FIG. 4) were generated. Newly generated antibodies were characterized, along with previous antibody MAB2859 from R&D Systems (Clone #767329). The epitope of each new anti-Siglec-6 antibody was mapped to Domain 1, 2, or 3 of the human Siglec-6 ECD and classified into bins (Table A). Melting temperature and affinity of binding to human Siglec-6 were also determined for each antibody, as shown in Table A.

TABLE A
Biochemical properties of anti-Siglec-6 mAbs.

| | | | | Melting |
|---|---|---|---|---|
| | Affinity | Domain | Epitope | Temperature |
| mAb Clone | (pM) | Mapping | Bin | (° C.) |
| AK15 | 1042 | 3 | — | 73.9 |
| AK14 | 72 | 3 | D | 74.35 |
| AK13 | 63 | 3 | D | 73.25 |
| AK12 | 87 | 3 | D | 74.5 |
| AK11 | 2 | 2 | E | 72.3 |
| AK10 | 84 | 2 | E | 73.15 |
| AK09 | 110 | 3 | D | 73.35 |
| AK08 | 261 | 3 | D | 74.6 |
| AK07 | 69 | 1 | C | 72.65 |
| AK06 | 24 | 1 | B | 72.75 |
| AK05 | 55 | 1 | B | 72.35 |
| AK04 | 1 | 1 | A | 72.5 |
| AK03 | 1 | 1 | A | 73.5 |
| AK02 | 7 | 1 | C | 74.45 |
| AK01 | 1 | 1 | A | 73.35 |
| R&D MAB2859 | 9 | 1 | A | 70.9 |

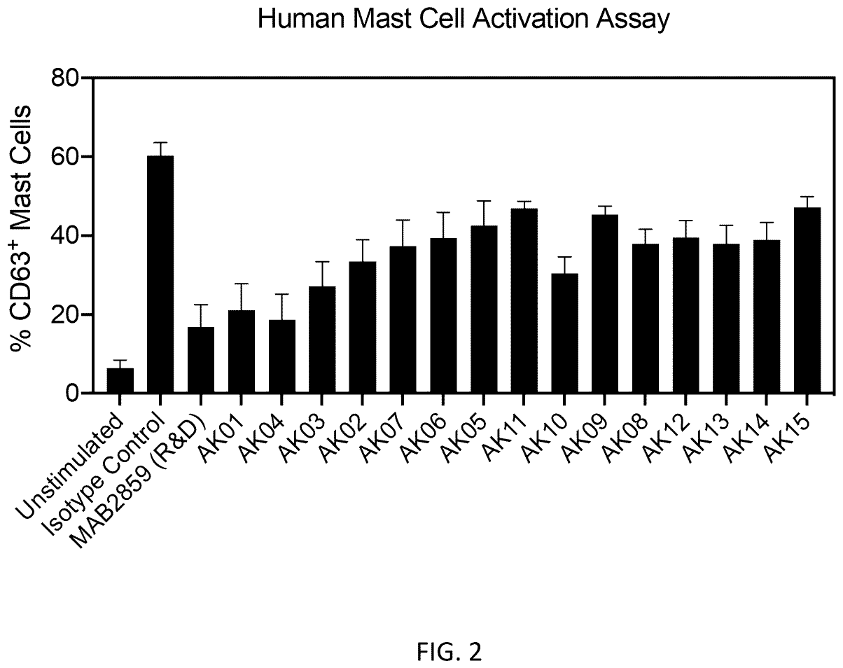

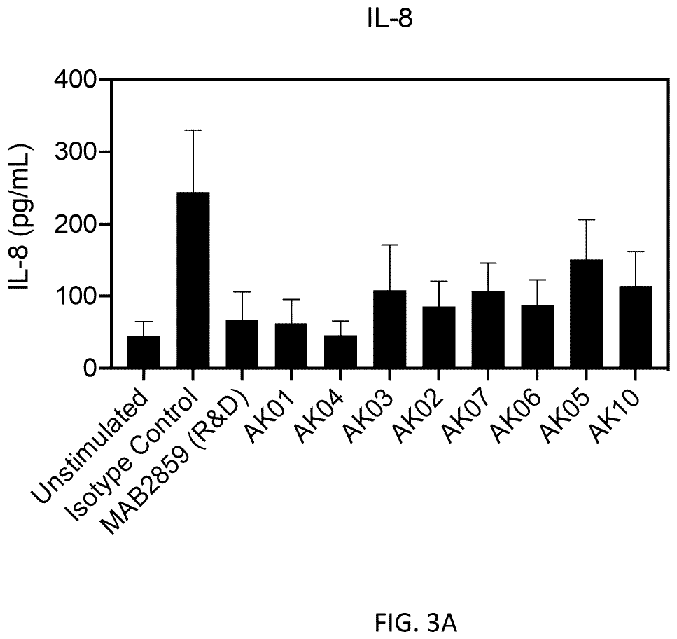

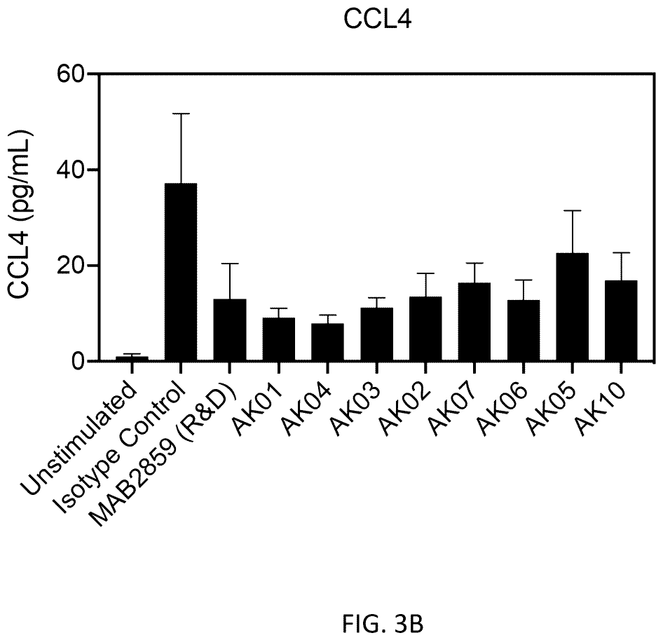

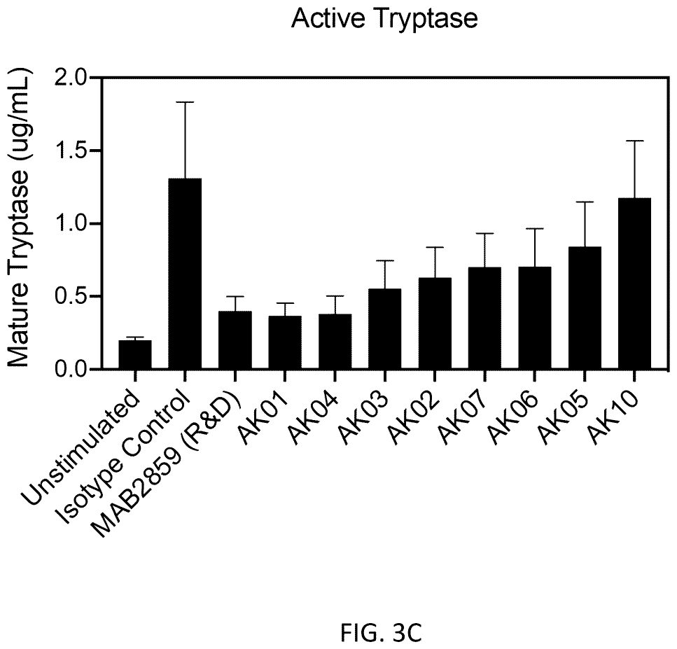

Next, the effect of antibody binding on mast cell activation was examined. Mast cell activation was assessed by flow cytometry using the activation marker CD63. All Siglec-6 mAbs tested were found to inhibit mast cell activation, compared to isotype control (FIG. 2). In addition, anti-Siglec-6 mAbs were found to inhibit production of cytokines, chemokines, and active proteases from human mast cells. All Siglec-6 mAbs tested reduced cytokines (e.g., IL-8 as shown in FIG. 3A) and chemokines (e.g., CCL4 as shown in FIG. 3B) from activated mast cells. All Siglec-6 mAbs tested also reduced tryptase release from activated mast cells (FIG. 3C).

Siglec-6 inhibition was found to be dependent on epitope location. For example, anti-Siglec-6 mAbs that bound to Domain 1, Bin A induced the strongest inhibition of mast cells, whereas mAbs that bound to Domain 3, Bin D, showed the weakest inhibition, as assessed by expression of CD63 and CD107a (FIG. 4A). Siglec-6 inhibition against FcRI-mediated mast cell inhibition was tested using representative Siglec-6 mAbs from each epitope bin. Human mast cells were derived from peripheral blood from healthy donors. Mast cells were stimulated with titrating concentrations of anti-FcεRI in the presence of anti-Siglec-6 mAbs or an isotype control (5 μg/mL) for 20 min at 37° C. Mast cell activation was assessed by flow cytometry using the surface marker CD63. Siglec-6 mAb inhibition was dependent on epitope bin, with Domain 1, bin A binders showing the most robust mast cell inhibition (FIG. 4B).

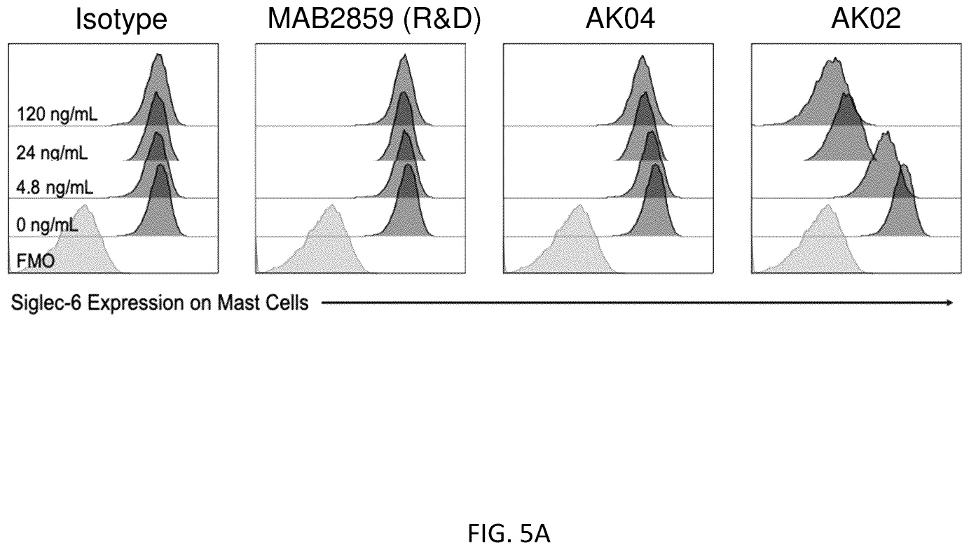

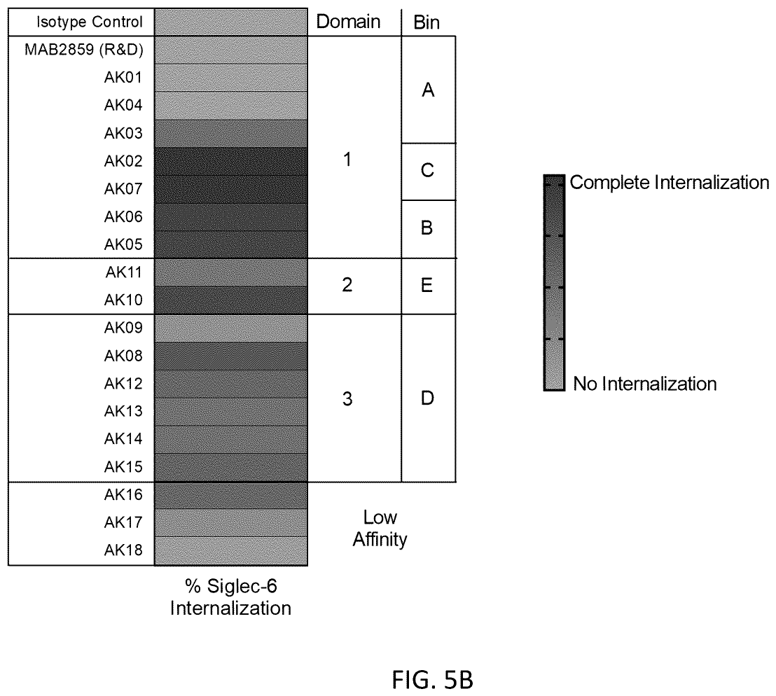

The effect of antibody binding on Siglec-6 internalization was also tested. Anti-Siglec-6 mAbs AK02 was found to induce dose-dependent Siglec-6 internalization on human mast cells, whereas AK04 and MAB2859 did not (FIG. 5A). Treatment with different anti-Siglec-6 antibodies led to a range of outcomes, from complete internalization to no internalization of Siglec-6 (FIG. 5B).

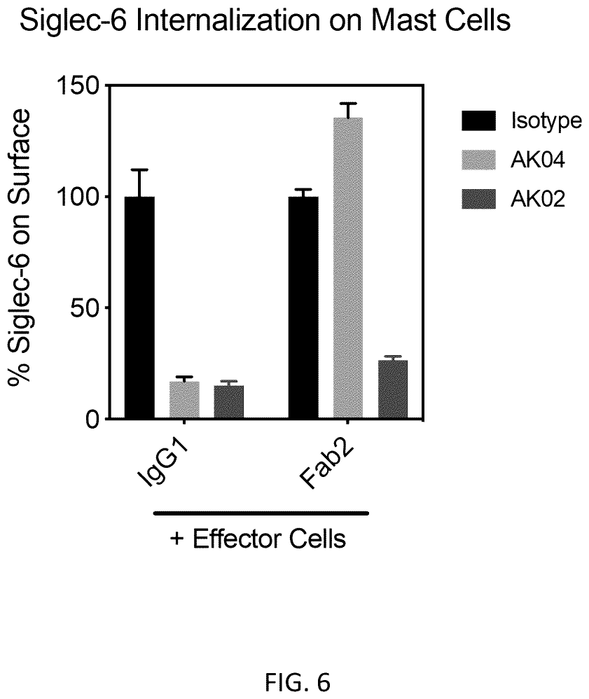

To examine whether Fc interactions were required for antibody-induced Siglec-6 internalization, human tissue mast cells were co-cultured with effector cells (human monocyte-derived macrophages) in the presence of 5 ug/mL IgG or Fab2 fragments of the Siglec-6 mAbs AK04 and AK02 or an isotype control for 18 h at 37° C. Internalization was measured by flow cytometry using a fluorescent-tagged Siglec-6 mAb that binds to a different epitope. AK04 IgG1, but not Fab2, induced internalization of Siglec-6 on human mast cells, demonstrating Fc-interaction is needed for receptor internalization induced by AK04 (FIG. 6). In contrast, both IgG and Fab2 formats of AK02 induced internalization of Siglec-6 receptor on human mast cells, indicating Fc-interactions are not required for internalization induced by AK02.

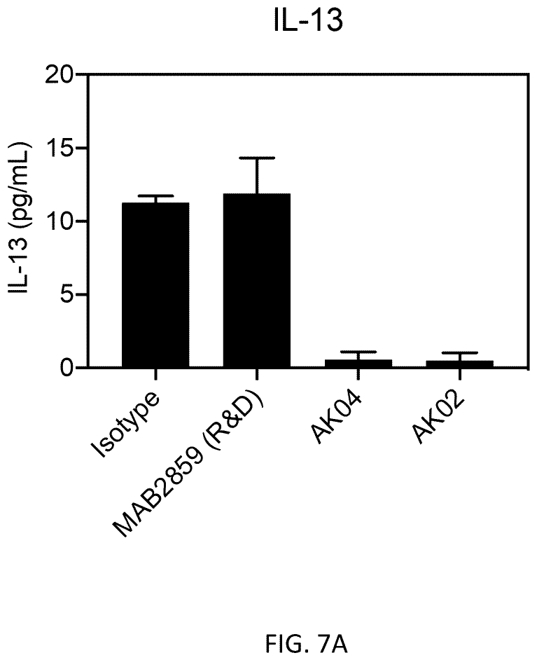

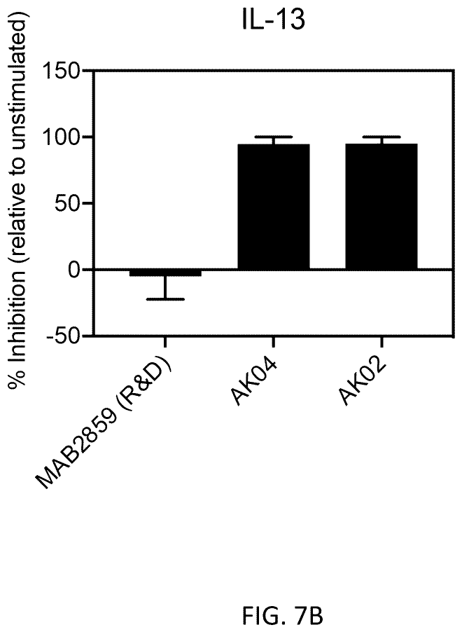

The effect of antibody binding on IL-13 production from activated human mast cells was also examined. IL-13 was measured by MSD in cell-free supernatants and was shown as absolute concentration (FIG. 7A) or percent inhibition (FIG. 7B). The Siglec-6 mAbs AK04 and AK02, but not MAB2859, inhibited production of IL-13 from activated mast cells.

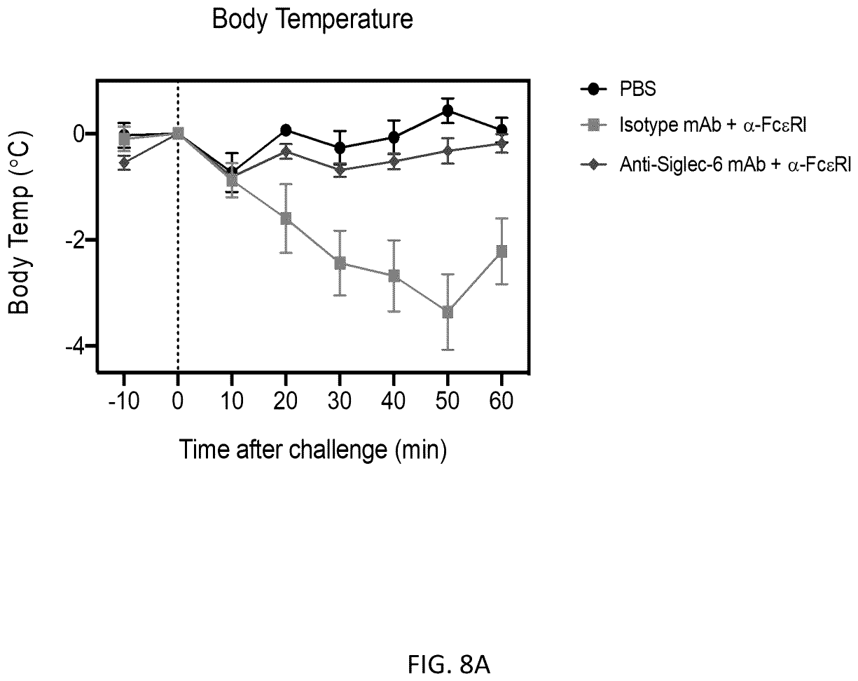

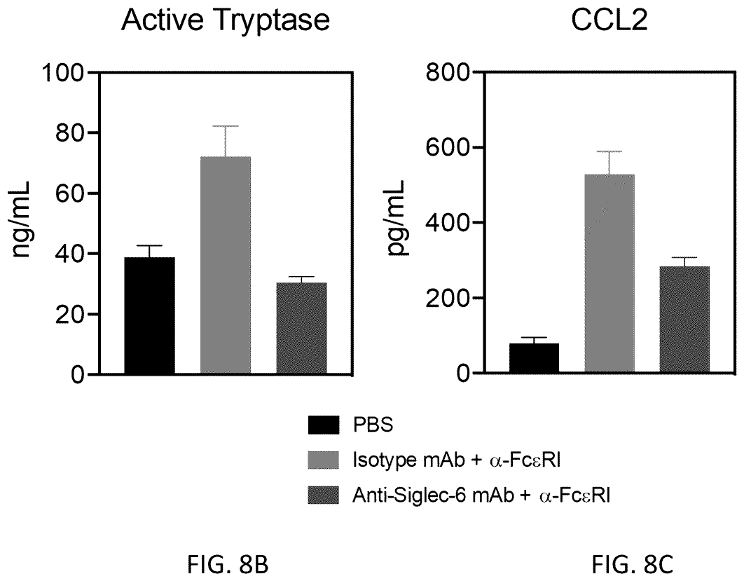

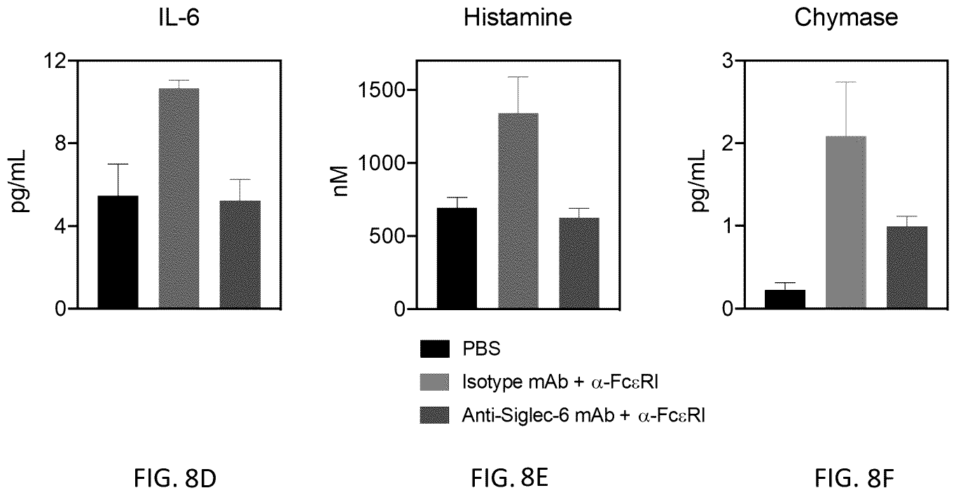

Activity of anti-Siglec-6 mAbs was also tested in the humanized mouse model described above. In FIG. 8A, mice receiving CRA-1 (squares) underwent severe anaphylaxis compared to PBS control mice (circles). Anti-Siglec-6 mAb (diamonds) completely inhibited IgE-mediated systemic anaphylaxis compared to isotype control-treated mice as measured by body temperature. Mice administered CRA-1 also displayed increased levels of active tryptase (FIG. 8B), CCL2 (FIG. 8C), IL-6 (FIG. 8D), histamine (FIG. 8E), and chymase (FIG. 8F), which are indicative of mast cell activation. Anti-Siglec-6 mAb treatment reduced systemic levels of these mediators, strongly suggestive of mast cell inhibition.

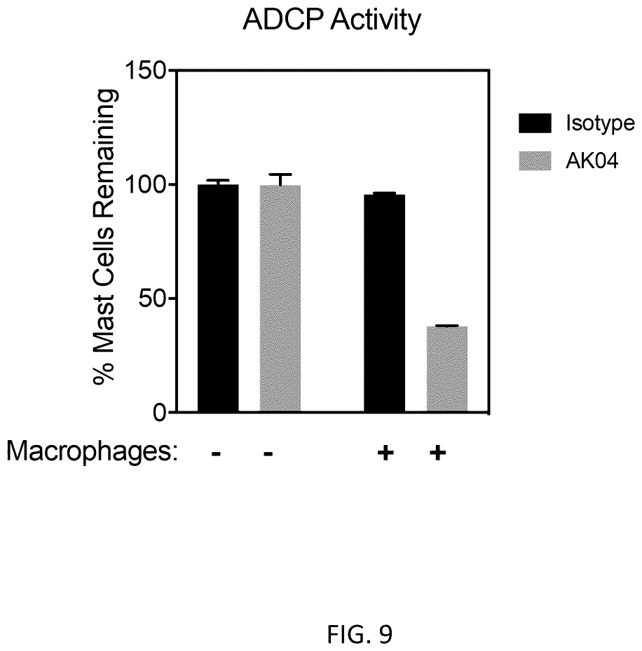

The anti-Siglec-6 mAb AK04 was also tested for antibody-dependent cellular phagocytosis (ADCP) activity against human mast cells. Human tissue mast cells were co-cultured with human monocyte-derived macrophages (1:50 ratio, respectively) in the presence of 5 ug/mL AK04 or an isotype control for 18 h at 37° C. Following overnight incubation, mast cell numbers were determined using flow cytometry and normalized to the number of mast cells in the isotype control-treated cells to obtain the percentage of mast cells remaining. AK04 reduced tissue mast cells only in the presence of macrophages, consistent with ADCP activity (FIG. 9).

Epitopes of selected anti-Siglec-6 antibodies were also mapped as described above. As shown in FIG. 10A, AK05 was determined to interact with the following amino acids on Siglec-6: 29, 30, 34, 38, 63, 64, 68, 74, 76, 99, 100, 103, 104, 106, and 114 (numbering according to the Siglec-6 ECD as shown in SEQ ID NO:1). As shown in FIG. 10B, AK04 was determined to interact with the following amino acids on Siglec-6: 26, 29, 30, 52, 64, 74, 75, 79, 98, 100, 104, 106, and 107 (numbering according to the Siglec-6 ECD as shown in SEQ ID NO:1). As shown in FIG. 10C, MAB2859 was determined to interact with the following amino acids on Siglec-6: 100, 103, 104, 106, 107, 109, 110, and 112 (numbering according to the Siglec-6 ECD as shown in SEQ ID NO:1).

Taken together, these results demonstrate the generation of new anti-Siglec-6 antibodies with high-affinity binding, covering a range of unique epitopes on Siglec-6 and differential properties with regard to modulating Siglec-6 surface expression and mast cell activation/inhibition.

### Example 2: Characterization of Anti-Siglec-6 Antibodies in a Siglec-6 Transgenic Mouse Model

To examine mast cell activation in vivo, Siglec-6 transgenic mice were injected intraperitoneally with PBS or 500 ng of recombinant mouse stem cell factor (SCF). Three hours later cells within the peritoneal cavity were collected by lavage with RPMI. A humanized anti-Siglec-6 mAb (comprising a VH domain comprising the sequence of SEQ ID NO:161 on a mIgG1 Fc and a VL domain comprising the sequence of SEQ ID NO:162) or isotype-matched control mAb were administered intraperitoneally at 5 mg/kg 1 h before SCF administration.

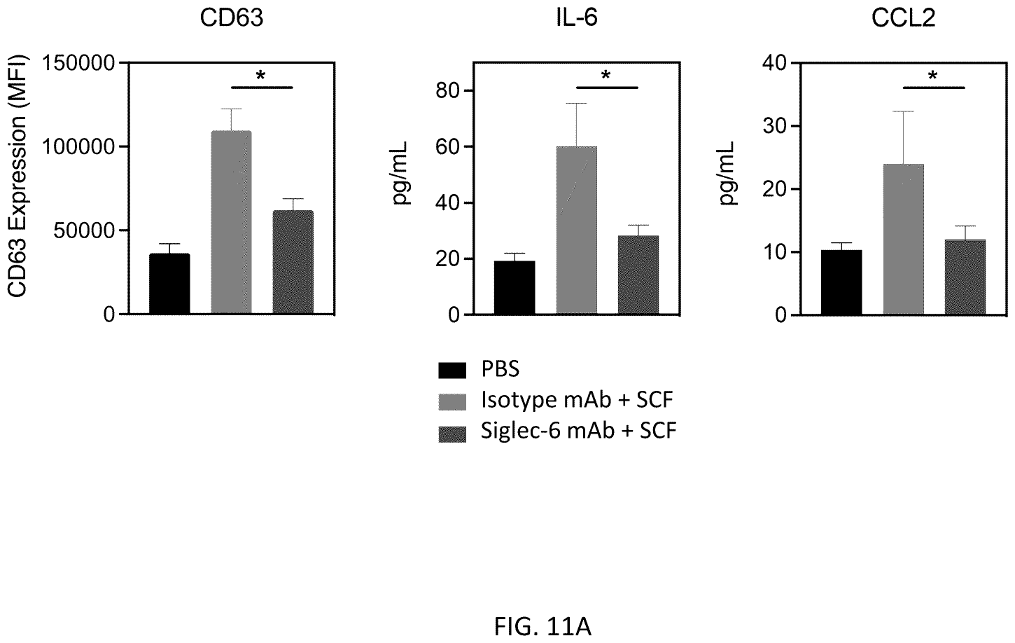

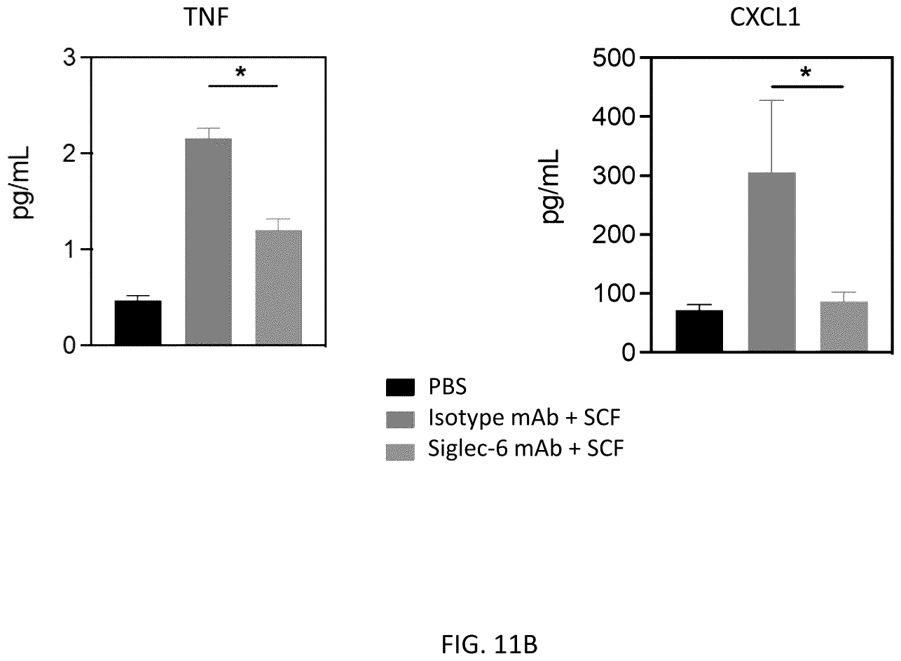

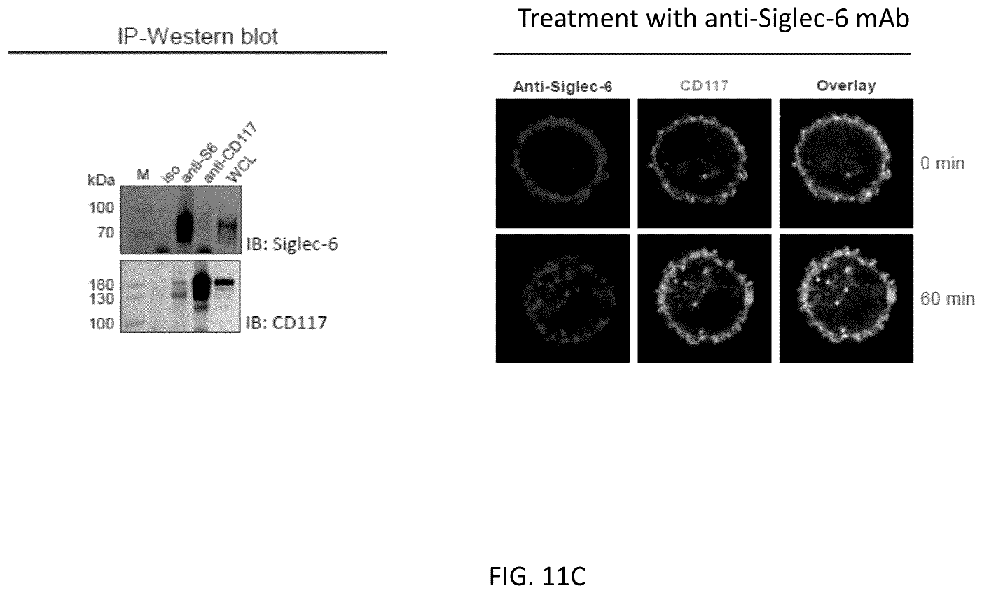

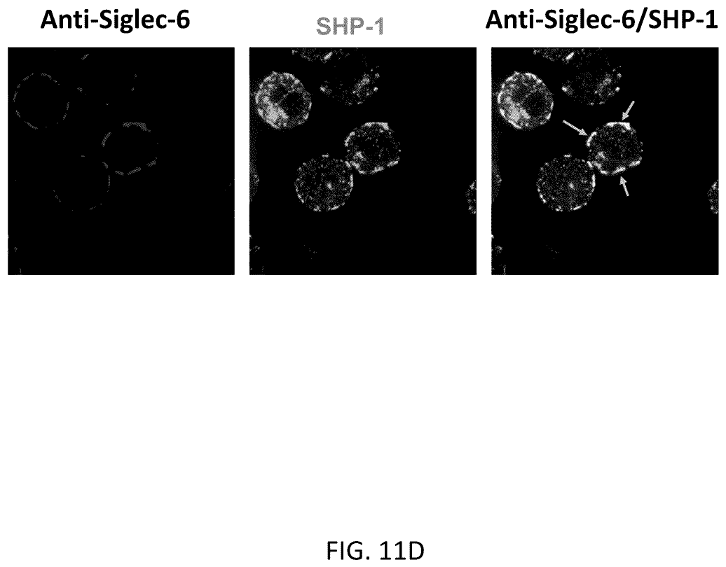

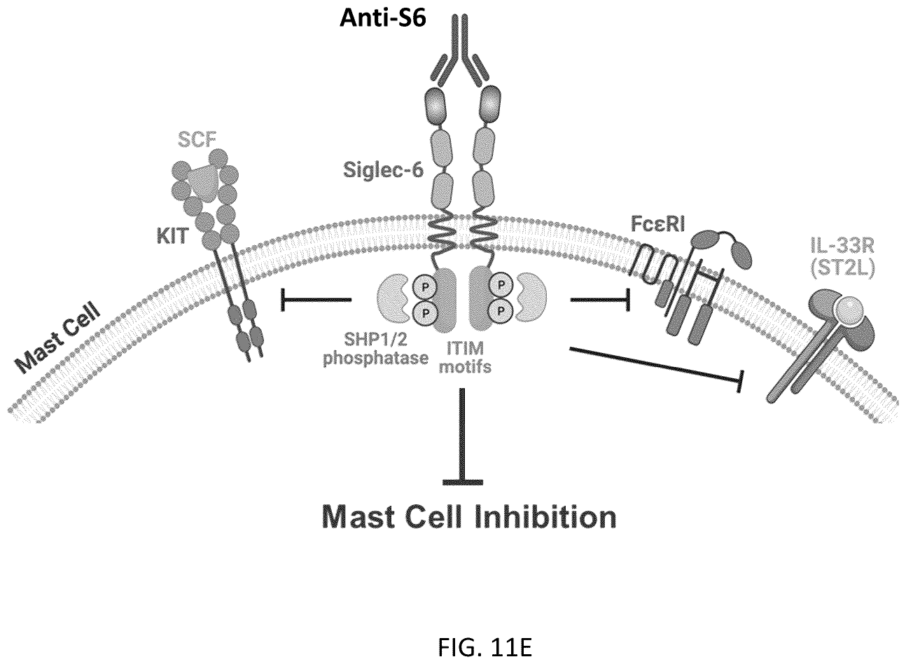

Anti-Siglec-6 mAb treatment was found to inhibit KIT (CD117)-mediated mast cell activation in this mouse model. Siglec-6 antibody treatment led to significantly lower CD63 expression than isotype control (FIG. 11A, left), as well as significantly lower cytokine and chemokine levels (e.g., IL-6, CCL2, TNF, and CXCL1; FIG. 11A, center and right and FIG. 11B). These data demonstrate that anti-Siglec-6 antibody treatment inhibited KIT- and IgE-mediated mast cell activation in Siglec-6 transgenic mice.

Further experiments were undertaken to interrogate protein interactions and signaling pathways effecting Siglec-6 inhibition in primary MCs and evaluate the breadth of inhibition of an agonistic Siglec-6 monoclonal antibody (mAb). Interactions of Siglec-6 with activating receptors and signaling molecules were evaluated biochemically and through confocal microscopy using primary MCs. The activity of an agonistic anti-Siglec-6 mAb was evaluated in vivo in the acute model of SCF-mediated inflammation in transgenic mice that express Siglec-6 on mouse MCs described above.

Siglec-6 was found to directly interact and colocalize with KIT/CD117 in MCs (FIG. 11C). In addition, the non-receptor inhibitory phosphatases Shp-1 and Shp-2 associated with Siglec-6 ITIMs upon phosphorylation (FIG. 11D, right). Mutation of both the proximal and distal ITIMs reduced phosphorylation and recruitment of Shp phosphatases. Importantly, engagement of Siglec-6 with an agonistic mAb induced microclusters containing inhibitory phosphatases and KIT/CD117, indicative of immunoregulatory synapses. Treatment with a Siglec-6 mAb inhibited SCF-mediated inflammation and the release of MC-specific proteases in Siglec-6 transgenic mice. These findings expand the understanding of MC inhibition through Siglec-6 and highlight the receptor as an attractive therapeutic target to broadly inhibit MCs in inflammatory diseases (FIG. 11E).

The effect of Siglec-6 antibody treatment was also examined in a mouse model of hapten-induced contact dermatitis. Siglec-6 transgenic mice were sensitized with 0.5% DNFB for 6 days, followed by 1.0% DNFB topical challenge. Twenty-four hours before DNFB challenge, mice were dosed with a humanized anti-Siglec-6 mAb (comprising a VH domain comprising the sequence of SEQ ID NO:161 on mouse IgG1 Fc and a VL domain comprising the sequence of SEQ ID NO:162) or isotype-matched control mAb. Skin inflammation was assessed 24 hours after DNFB topical challenge.

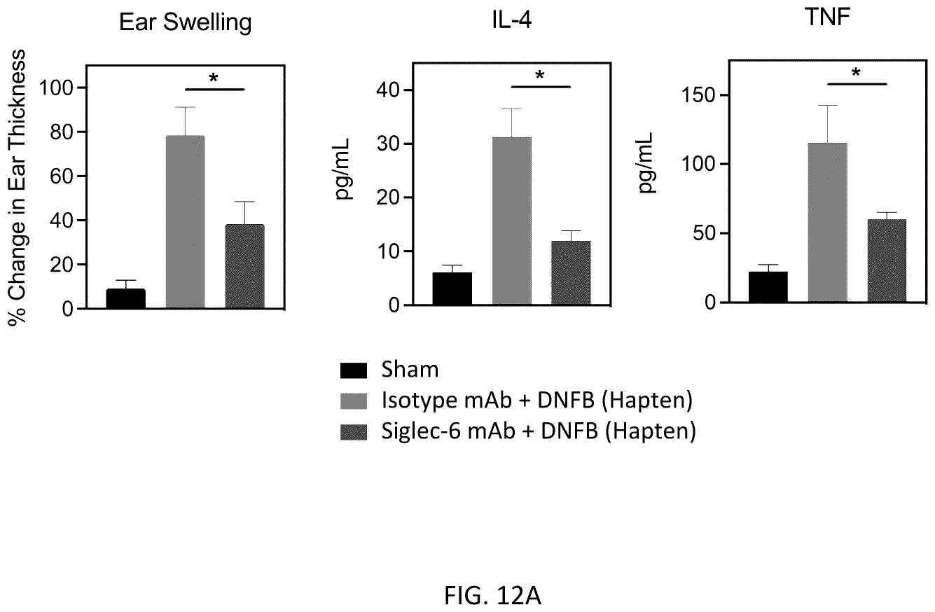

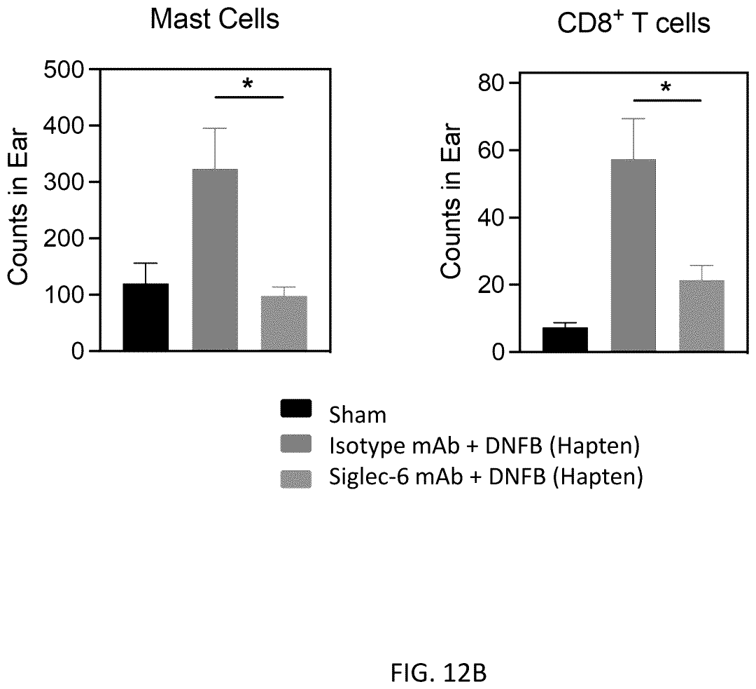

Anti-Siglec-6 mAb treatment was found to inhibit allergic contact dermatitis in this mouse model. Siglec-6 antibody treatment led to significantly lower skin inflammation than isotype control, as measured by ear swelling (FIG. 12A, left), counts of mast cells in ear tissue (FIG. 12B, left), and counts of CD8+ T cells in ear tissue (FIG. 12B, right). Cytokine levels were also assessed in ear tissue cultured ex vivo. Levels of IL-4 (FIG. 12A, center) and TNF (FIG. 12A, right) were also significantly lower in the anti-Siglec-6 treatment group, as compared to isotype control. These results demonstrate that anti-Siglec-6 antibody treatment reduced skin inflammation in this contact dermatitis model via mast cell inhibition.

### Example 3: Characterization of Anti-Siglec-6 Antibodies in Human Lung Tissue and Humanized Mice

The effect of Siglec-6 antibody on activation of mast cells from human lung tissue was tested. Human lung tissues (n=3) were enzymatically and mechanistically digested into single cells. Single cell suspensions were incubated unstimulated or with an agonistic anti-FcεRI antibody (CRA-1 clone, 10 μg/mL) in the presence of increasing concentrations of a humanized anti-Siglec-6 mAb (comprising a VH domain comprising the sequence of SEQ ID NO:161 and a VL domain comprising the sequence of SEQ ID NO:162) for 30 minutes at 37° C. Following incubation, mast cell activation was evaluated by CD63 expression using flow cytometry.

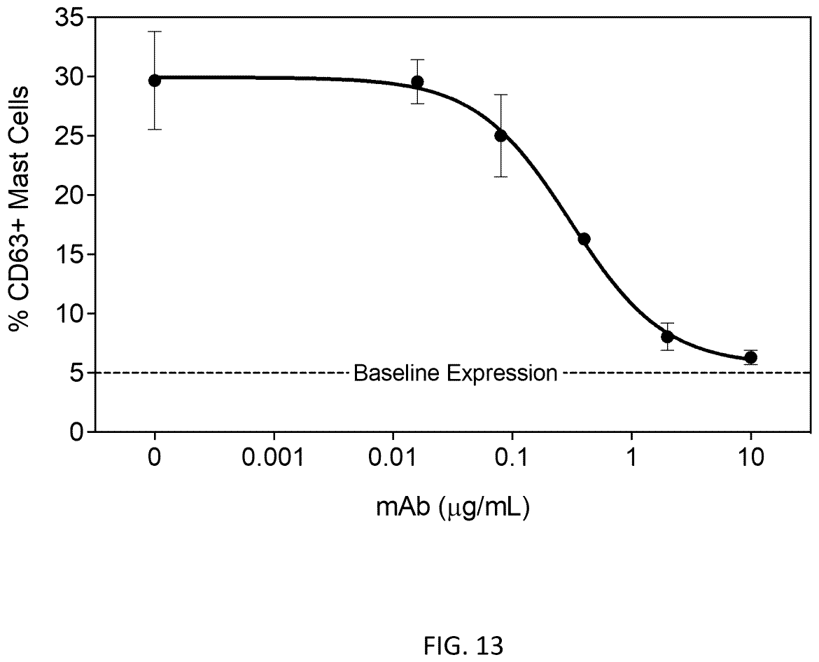

As shown in FIG. 13, Siglec-6 mAb potently inhibited IgE-mediated activation of human tissue mast cells.

Next, the importance of antibody Fc region on reduction of human tissue mast cells was examined in vivo using humanized mice. Humanized mice (SGM3-BLT) were dosed every 3 days with an Isotype control mAb, humanized anti-Siglec-6 mAb (comprising a VH domain comprising the sequence of SEQ ID NO:161 and a VL domain comprising the sequence of SEQ ID NO:162), or equimolar equivalent of Siglec-6 F(ab′)2 (same humanized mAb) for 14 days (n=5-6 mice/group). On day 14, mice were taken down and mast cells in the peritoneal cavity and lung were analyzed using flow cytometry.

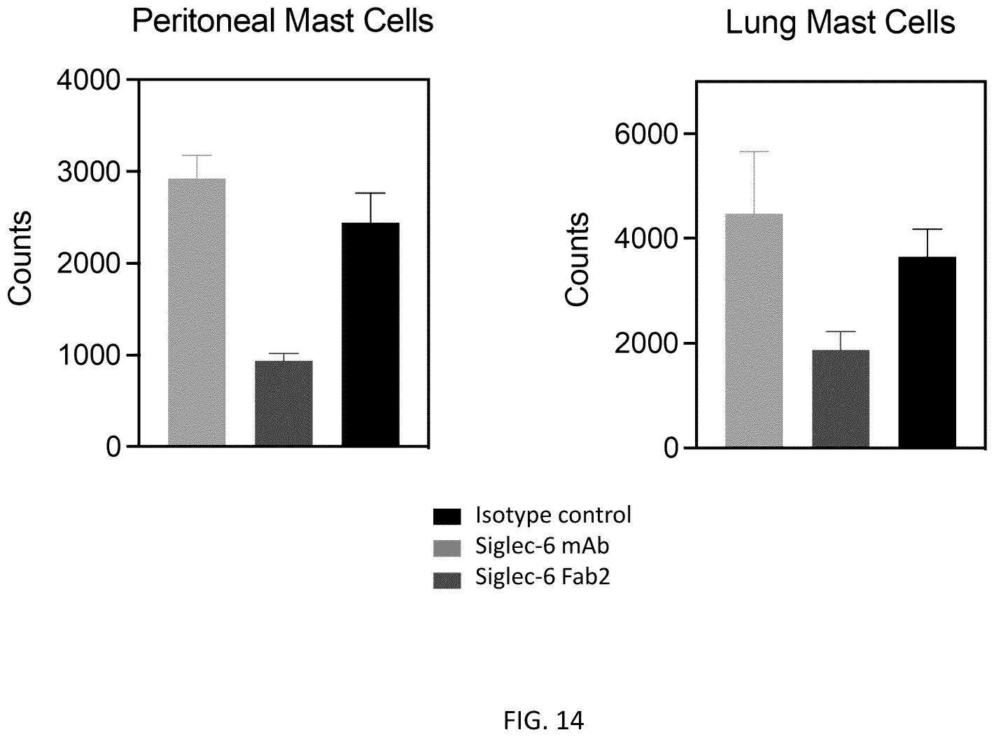

FIG. 14 shows that mice treated with a Siglec-6 mAb showed reduced mast cell numbers compared to isotype control-treated mice. Siglec-6 F(ab′)2-treated mice showed normal levels of mast cells compared to Siglec-6 mAb treated mice, suggesting the Fc region of the Siglec-6 mAb is needed for mast cell reduction activity.

### Example 4: Characterization of Anti-Siglec-6 Antibody in a Murine Model of IL-33-Driven Acute Skin Inflammation

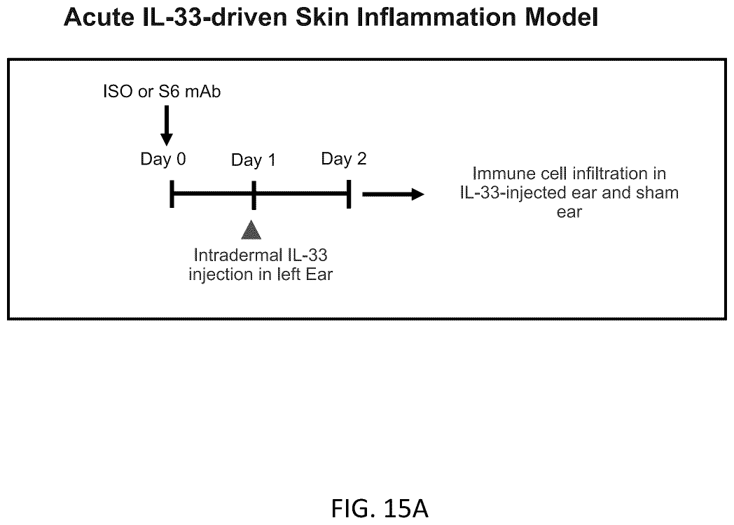

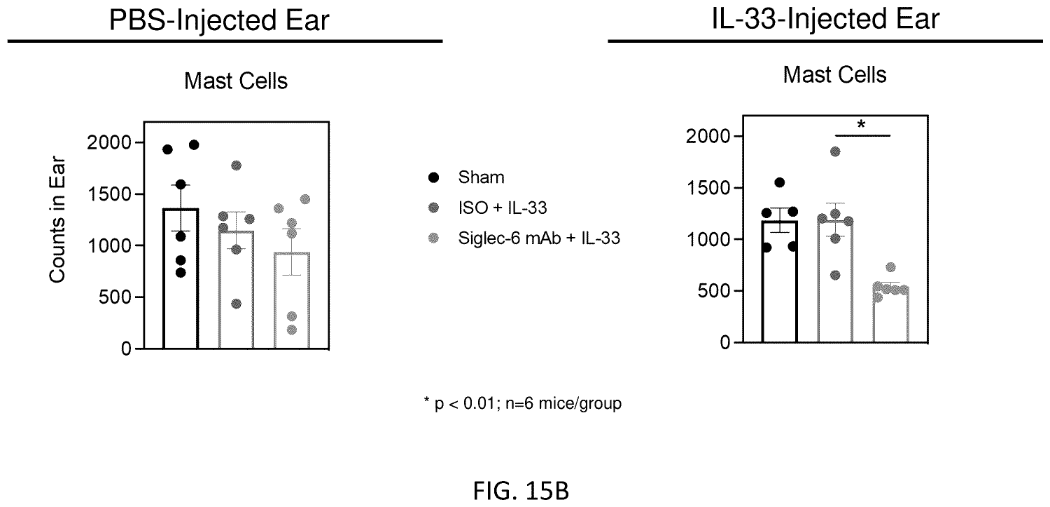

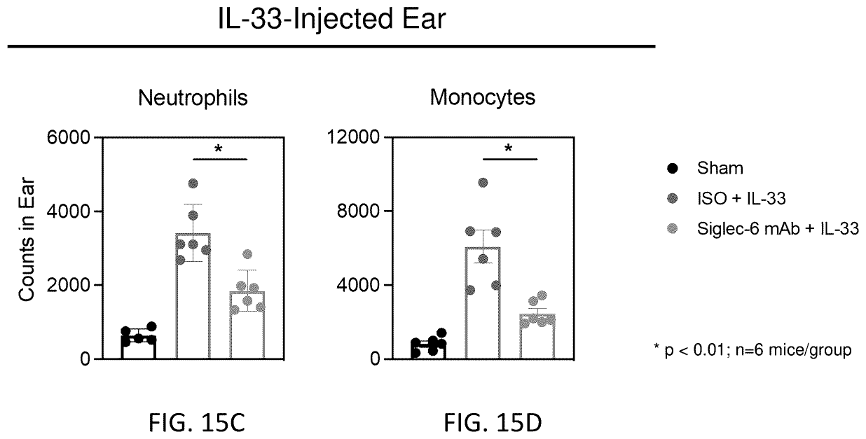

The effect of Siglec-6 antibody on acute skin inflammation was tested. Acute skin inflammation was induced in mice in an IL-33-driven model (FIG. 15A). Mice were administered 10 mg/kg humanized anti-Siglec-6 mAb (comprising a VH domain comprising the sequence of SEQ ID NO:161 on mIgG2a Fc and a VL domain comprising the sequence of SEQ ID NO:162) or isotype control via intravenous injection. The next day, mice received 250 ng recombinant mouse IL-33 in the left ear and PBS in the right ear (sham) via intradermal injection. Local inflammation in the ear was measured by quantifying immune cell infiltration in the ear 24 hours later by flow cytometry.

Anti-Siglec-6 mAb treatment inhibited IL-33-mediated inflammation and reduced mast cell (MC) numbers (FIG. 15B). Mice dosed with anti-Siglec-6 mAb also showed significantly reduced neutrophils (FIG. 15C) and monocytes (FIG. 15D). Anti-Siglec-6 mAb treatment reduced MC numbers specifically in ears that received IL-33 but not in PBS-injected sham control ears, demonstrating that anti-Siglec-6 mAb treatment reduced MC numbers only in sites of local inflammation.

### Example 5: Characterization of Anti-Siglec-6 Antibody in a Murine Model of Allergic Gastrointestinal Disease in Siglec-6 Transgenic Mice

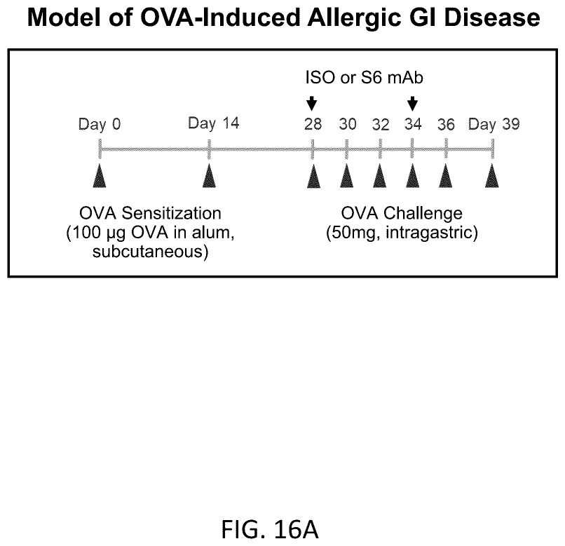

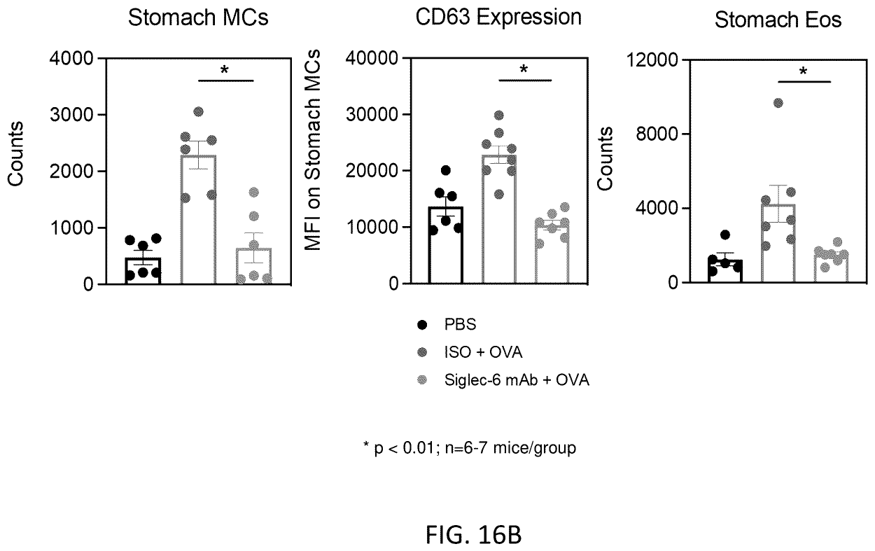

The effect of Siglec-6 antibody on allergic gastrointestinal disease was tested. Allergic gastrointestinal disease was induced in a mouse model via ovalbumin (OVA) peptide administration (FIG. 16A). Mice were sensitized twice, 2 weeks apart, with 100 μg of OVA in the presence of 1 mg of aluminum potassium sulfate adjuvant by subcutaneous injection. On Day 28, mice were held in the supine position three times a week (every other day) and orally administered 250 μL of sterile saline that contained up to 50 mg of OVA. Before each intragastric challenge, mice were deprived of food for 3-4 hours with the aim of limiting antigen degradation in the stomach. Mice received intraperitoneal (IP) injection of 10 mg/kg of either an anti-Siglec-6 mAb (humanized mAb comprising a VH domain comprising the sequence of SEQ ID NO:161 on mIgG1 Fc and a VL domain comprising the sequence of SEQ ID NO:162) or isotype-matched control (mIgG1) mAb on days 28 and 34. Mice were taken down on day 39. Immune cell numbers and mast cell activation were determined by flow cytometry and mediators were quantified in serum using Meso Scale Discovery (MSD).

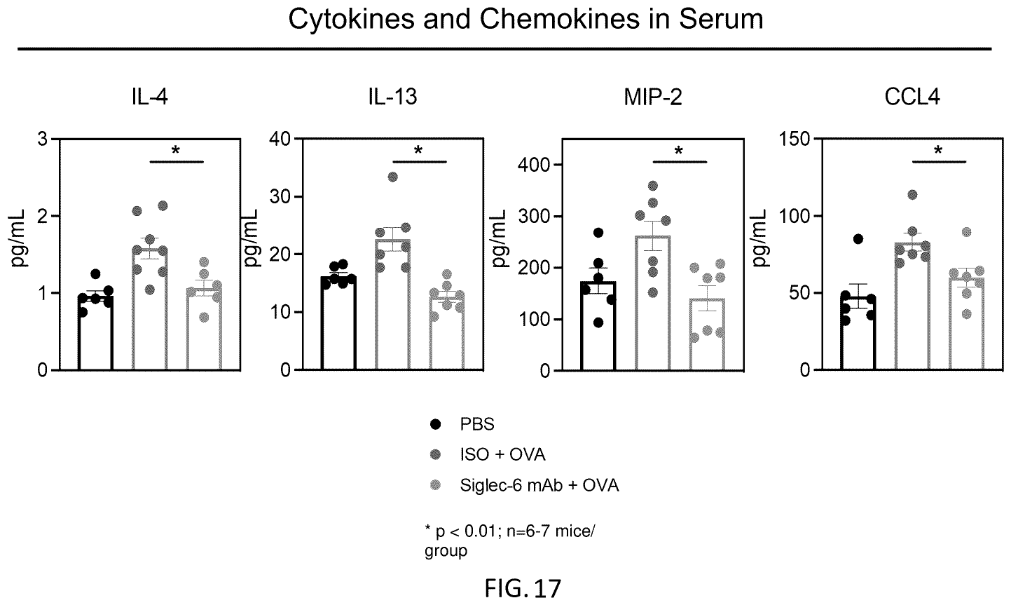

OVA-sensitized and challenged mice showed increased mast cell (MC) infiltration and degranulation in the stomach, as well as increased numbers of infiltrating eosinophils in the stomach. However, mice that were treated with humanized anti-Siglec-6 mAb with the mIgG1 Fc demonstrated a significant reduction in these metrics of OVA-induced inflammation (FIG. 16B). Anti-Siglec-6 mAb treatment also led to a reduction in the production of inflammatory mediators measured in the serum (FIG. 17), including the cytokines IL-4 and IL-13 and the chemokines MIP-2 and CCL4. Taken together, these results indicate that anti-Siglec-6 mAb can reduce effectively measures of gastrointestinal inflammation that are caused by chronic antigen exposure.

### Example 6: Anti-Siglec-6 Antibody Treatment Reduces Skin Inflammation Mediated by MRGPRX2 Agonist LL37

The effect of Siglec-6 antibody (humanized anti-Siglec-6 mAb comprising a VH domain comprising the sequence of SEQ ID NO:161 on mIgG2a Fc and a VL domain comprising the sequence of SEQ ID NO:162) on skin inflammation in an MRGPRX2 agonist LL37 model was tested. MRGPRX2 encodes a MAS related GPR family member X2 which is involved in mast cell degranulation, and LL37 is a multifunctional antimicrobial peptide agonist of MRGPRX2.

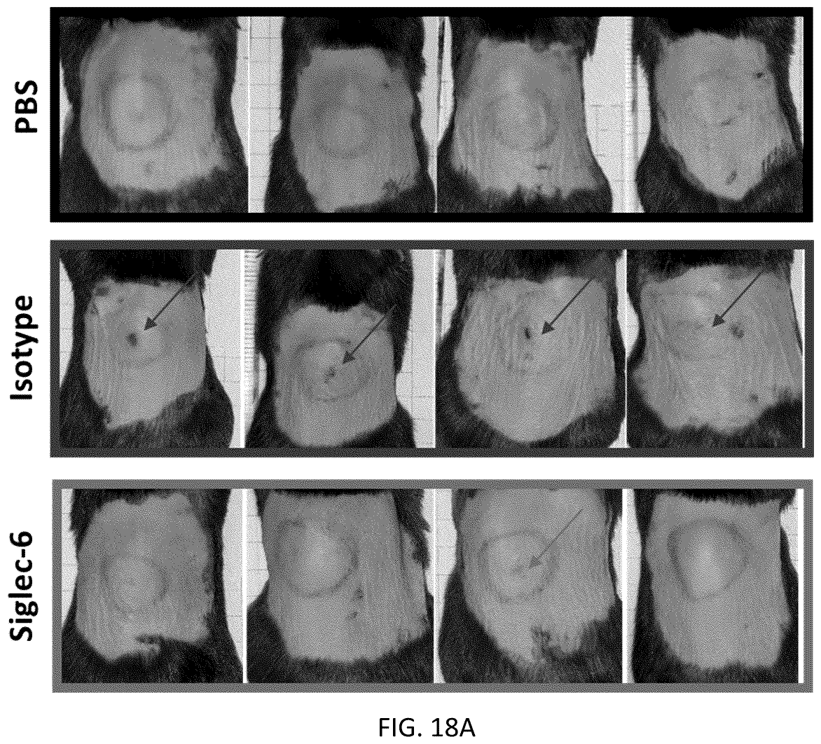

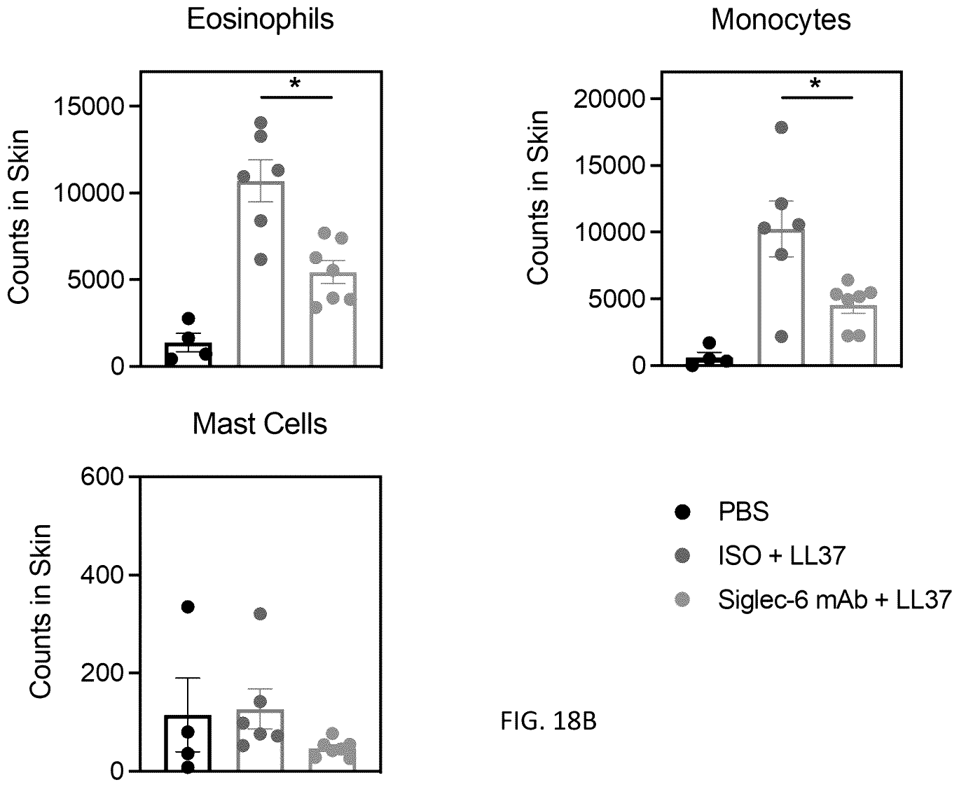

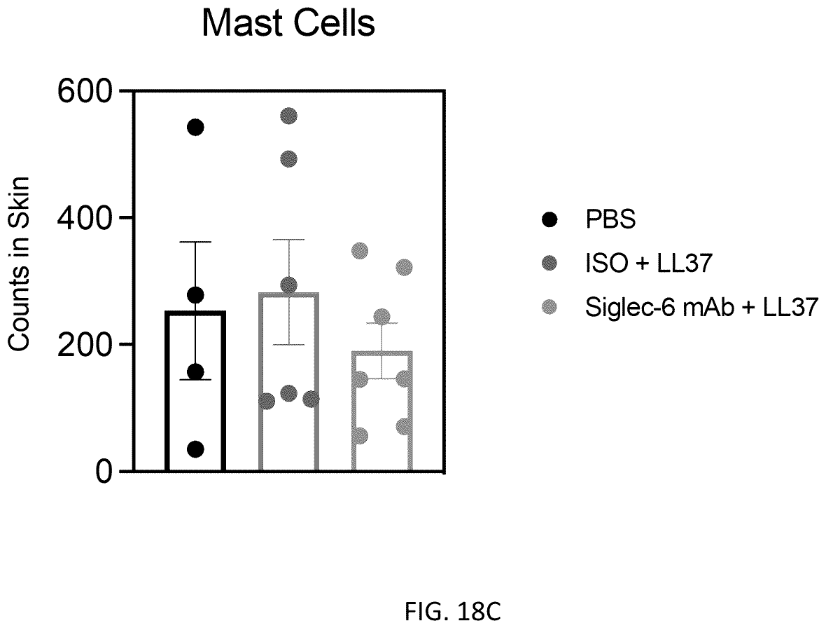

LL37 treatment combined with isotype control antibody led to skin inflammation and appearance of lesions (FIG. 18A, “Isotype”), while treatment with PBS only did not (FIG. 18A, “PBS”). Cell counts revealed increases in eosinophils and monocytes in lesional skin (FIG. 18B), but not non-lesional skin (FIG. 18C). However, LL37-induced skin lesions were reduced in appearance in Siglec-6 mAb-treated mice (FIG. 18A, “Siglec-6”) with concomitant decreases in MC, eosinophil, and monocyte numbers (FIG. 18B).

### Example 7: Effect of Fc Function on Anti-Siglec-6-Mediated Mast Cell Inhibition

This Example demonstrates the influence of Fc function on the ability of anti-Siglec-6 mAbs to inhibit mast cell activation.

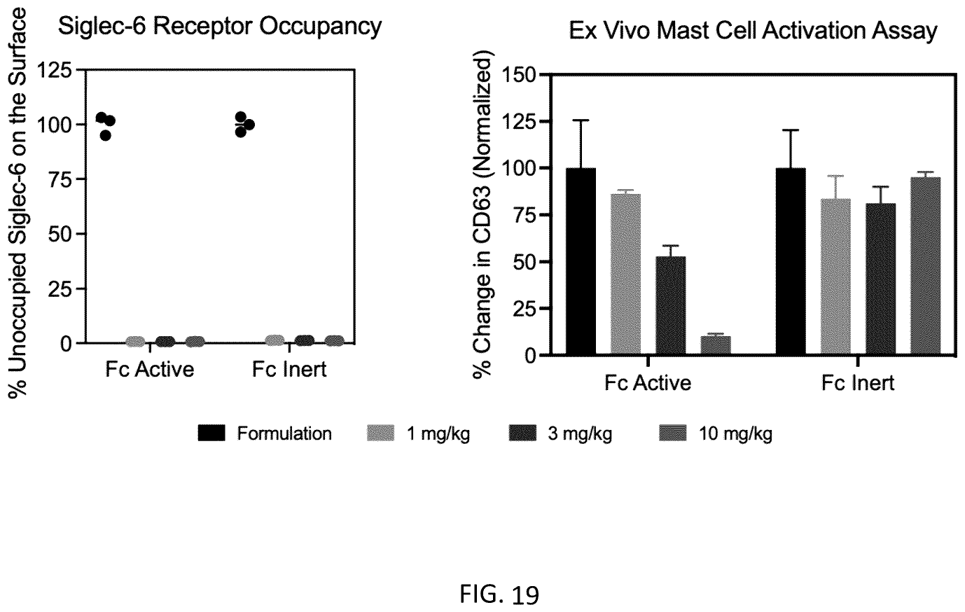

Mice were intravenously administered a single dose of the same humanized anti-Siglec-6 mAb (comprising a VH domain comprising the sequence of SEQ ID NO:161 and a VL domain comprising the sequence of SEQ ID NO:162) having either an active mIgG2a Fc or inert mIgG2a Fc (LALA-PG mutations; L234A/L235A/P329G) at 1 mg/kg, 3 mg/kg, 10 mg/kg, or formulation buffer for 7 days. On day 7, mice were sacrificed, and peritoneal mast cells were isolated followed by assessment of Siglec-6 receptor occupancy and mast cell activation. Mice administered Fc active or Fc inert anti-Siglec-6 mAb mIgG2a at all doses showed full receptor occupancy (FIG. 19; left).

To assess mast cell inhibition, peritoneal mast cells were activated through the FcεRI pathway. Mast cell activation was assessed by quantifying the degranulation marker CD63 by flow cytometry. Mice treated with Fc active humanized anti-Siglec-6 mIgG2a at 3 and 10 mg/kg showed inhibition of mast cell activation, compared to mice administered formulation buffer (FIG. 19; right). In contrast, mice administered Fc inert humanized anti-Siglec-6 mIgG2a showed minimal mast cell inhibition at all doses evaluated. These findings demonstrate an active Fc is required to induce mast cell inhibition with humanized anti-Siglec-6 mAb.

### Example 8: Humanization of Anti-Siglec-6 mAbs

Non-human monoclonal antibodies have been shown to induce human immune responses, which result in neutralization of administered antibody and limits the application of such antibodies in treatment of human diseases. To overcome this problem, antibody humanization has been developed to eliminate or reduce the immunogenicity of antibodies. The humanization process identifies and retains the amino acids from the reference antibody that are required for high affinity antigen-binding and attempts to change all other residues to human germline. The process generates antibodies with variable (V)-region sequences that are almost identical to human germline sequence while retaining the epitope specificity and affinity of a reference antibody.

To improve the tolerability of AK04 in humans, a humanization strategy was developed to replace the mouse germline residues with human germline residues and retain the high affinity and biological activity of the reference antibody. The heavy chain complementarity-determining region (HCDR) and light chain complementarity-determining region (LCDR) were incorporated into the human germline sequences Vh4-59 and VkI-39 for the heavy and light chains, respectively. Diverse sequence libraries containing all possible combinations for the mutated framework positions were constructed in a single-chain variable fragment (scFv) phage display library and selected for high affinity binding to the antigen. Through this process, several framework sequence combinations were identified that retained high affinity binding to Siglec-6 and showed high identity to human germline. One set of humanized V-region sequences was selected (AK19) based off comparable binding affinity to Siglec-6 as the parental mouse antibody. The AK19 heavy chain was further humanized to generate AK20 and AK21 by diversifying several positions within the HCDR1 and HCDR2 to improve affinity and increase the percent identity to human germline. Fabs were generated and monovalent affinities were determined by biolayer interferometry for AK19, AK20, and AK21 and compared to the reference AK04. The three humanized Fabs showed comparable affinity as the reference Fab to Siglec-6, indicating the framework and HCDR2 changes were not critical for antigen binding (Table B). Additionally, the percent identify to human was also determined with AK20 and AK21 showing the highest homology to the human germline amino acid sequence (Table B).

TABLE B
Antigen binding properties and percentage of
human germline for humanized AK04 variants.

| | | | | % Germline vs | % Germline vs |
|---|---|---|---|---|---|
| Sequence | KD (M) | Kon (1/Ms) | Koff (1/s) | Vh4-59 | VkIII L25 |
| AK04 Reference | 7.5E−11 | 8.6E+05 | 6.5E−05 | 66.7 | 68.6 |
| AK19 | 3.2E−11 | 8.3E+05 | 2.6E−05 | 84.4 | 89.9 |
| AK20 | 1.2E−10 | 7.5E+05 | 9.1E−05 | 87.1 | 89.9 |
| AK21 | 4.0E−11 | 9.2E+05 | 3.7E−05 | 89.9 | 89.9 |

AK20 and AK21 were further evaluated in a forced degradation stability study. AK20 and AK21 (both using a wild-type human IgG1 Fc region with normal fucosylation) were incubated for 2 weeks at elevated temperatures and low pH followed by assessment of activity using a Siglec-6 binding ELISA. AK21 showed reduced activity at stressed conditions compared to AK20, suggesting AK20 is more stable.

TABLE C
Anti-Siglec-6 potency during 2-week forced degradation study.

| | EC50 at | EC50 at | Potency¹ at | Potency¹ at |
|---|---|---|---|---|
| Sequence # | 1 Week | 2 Weeks | 1 Week (%) | 2 Weeks (%) |
| AK20 | 6.5 ng/mL | 6.4 ng/mL | 103.4 | 104.8 |
| AK21 | 9.1 ng/mL | 9.3 ng/mL | 78.6 | 76.5 |

¹Potency was determined at 1 and 2 weeks using the EC50 of the reference material at Day 0.

Variable domain sequences for the three humanized anti-Siglec-6 variants are provided below. HVR sequences according to Chothia definition are underlined.

AK19 Vh
(SEQ ID NO: 159)
QVQLQESGPGLVKPSETLSLTCTVSGFSLTSYGVSWIRQPPGKGLEWIG
VIWHDGSTSYHSTLISRVTISRDTSKNQVSLKLSSVTAADTAVYYCASD
GYSGTFAYWGQGTLVTVSS
AK19Vk
(SEQ ID NO: 160)
QIVVTQSPATLSLSPGERATLSCTASSSVSSSYLHWYQQKPGQAPRLLI
YSTSILASGIPARFSGSGSGTDFTLTISSLQPEDFAVYYCHQYHRSPYT
FGQGTKLEIK
AK20 Vh
(SEQ ID NO: 161)
QVQLQESGPGLVKPSETLSLTCTVSGFSLTSYGVSWIRQPPGKGLEWIG
VIWHDGSTSYHPSLKSRVTISRDTSKNQVSLKLSSVTAADTAVYYCASD
GYSGTFAYWGQGTLVTVSS
AK20 Vk
(SEQ ID NO: 162)
QIVVTQSPATLSLSPGERATLSCTASSSVSSSYLHWYQQKPGQAPRLLI
YSTSILASGIPARFSGSGSGTDFTLTISSLQPEDFAVYYCHQYHRSPYT
FGQGTKLEIK
AK21 Vh
(SEQ ID NO: 163)
QVQLQESGPGLVKPSETLSLTCTVSGFSLSSYGVSWIRQPPGKGLEWIG
VIWHDGSTNYNPSLKSRVTISRDTSKNQVSLKLSSVTAADTAVYYCASD
GYSGTFAYWGQGTLVTVSS
AK21 Vk
(SEQ ID NO: 164)
QIVVTQSPATLSLSPGERATLSCTASSSVSSSYLHWYQQKPGQAPRLLI
YSTSILASGIPARFSGSGSGTDFTLTISSLQPEDFAVYYCHQYHRSPYT
FGQGTKLEIK

1. A humanized antibody that binds to human Siglec-6, wherein the antibody binds to Domain 1 of an extracellular domain of human Siglec-6.

2. (canceled)

3. An antibody that binds to human Siglec-6, wherein the antibody binds to Domain 2 of an extracellular domain of human Siglec-6.

4. (canceled)

5. An antibody that binds to human Siglec-6, wherein the antibody binds to Domain 3 of an extracellular domain of human Siglec-6.

6-27. (canceled)

28. The antibody of claim 1, wherein the antibody comprises a heavy chain variable (VH) region and a light chain variable (VL) region; wherein:
(a) the VH region comprises an HVR-H1 comprising the amino acid sequence of SEQ ID NO:89, an HVR-H2 comprising the amino acid sequence of SEQ ID NO:90, and an HVR-H3 comprising the amino acid sequence of SEQ ID NO:91, and the VL region comprises an HVR-L1 comprising the amino acid sequence of SEQ ID NO:92, an HVR-L2 comprising the amino acid sequence of SEQ ID NO:93, and an HVR-L3 comprising the amino acid sequence of SEQ ID NO:94;
(b) the VH region comprises an HVR-H1 comprising the amino acid sequence of SEQ ID NO:17, an HVR-H2 comprising the amino acid sequence of SEQ ID NO:18, and an HVR-H3 comprising the amino acid sequence of SEQ ID NO:19, and the VL region comprises an HVR-L1 comprising the amino acid sequence of SEQ ID NO:20, an HVR-L2 comprising the amino acid sequence of SEQ ID NO:21, and an HVR-L3 comprising the amino acid sequence of SEQ ID NO:22;
(c) the VH region comprises an HVR-H1 comprising the amino acid sequence of SEQ ID NO:83, an HVR-H2 comprising the amino acid sequence of SEQ ID NO:84, and an HVR-H3 comprising the amino acid sequence of SEQ ID NO:85, and the VL region comprises an HVR-L1 comprising the amino acid sequence of SEQ ID NO:86, an HVR-L2 comprising the amino acid sequence of SEQ ID NO:87, and an HVR-L3 comprising the amino acid sequence of SEQ ID NO:88;
(d) the VH region comprises an HVR-H1 comprising the amino acid sequence of SEQ ID NO:5, an HVR-H2 comprising the amino acid sequence of SEQ ID NO:6, and an HVR-H3 comprising the amino acid sequence of SEQ ID NO:7, and the VL region comprises an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO: 10;
(e) the VH region comprises an HVR-H1 comprising the amino acid sequence of SEQ ID NO:11, an HVR-H2 comprising the amino acid sequence of SEQ ID NO:12, and an HVR-H3 comprising the amino acid sequence of SEQ ID NO:13, and the VL region comprises an HVR-L1 comprising the amino acid sequence of SEQ ID NO: 14, an HVR-L2 comprising the amino acid sequence of SEQ ID NO:15, and an HVR-L3 comprising the amino acid sequence of SEQ ID NO: 16;
(f) the VH region comprises an HVR-H1 comprising the amino acid sequence of SEQ ID NO:77, an HVR-H2 comprising the amino acid sequence of SEQ ID NO:78, and an HVR-H3 comprising the amino acid sequence of SEQ ID NO:79, and the VL region comprises an HVR-L1 comprising the amino acid sequence of SEQ ID NO:80, an HVR-L2 comprising the amino acid sequence of SEQ ID NO:81, and an HVR-L3 comprising the amino acid sequence of SEQ ID NO: 82;
(g) the VH region comprises an HVR-H1 comprising the amino acid sequence of SEQ ID NO:71, an HVR-H2 comprising the amino acid sequence of SEQ ID NO:72, and an HVR-H3 comprising the amino acid sequence of SEQ ID NO:73, and the VL region comprises an HVR-L1 comprising the amino acid sequence of SEQ ID NO:74, an HVR-L2 comprising the amino acid sequence of SEQ ID NO:75, and an HVR-L3 comprising the amino acid sequence of SEQ ID NO:76; or
(h) the VH region comprises an HVR-H1 comprising the amino acid sequence of SEQ ID NO:168, an HVR-H2 comprising the amino acid sequence of SEQ ID NO:169, and an HVR-H3 comprising the amino acid sequence of SEQ ID NO:7, and the VL region comprises an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO:10.

29-35. (canceled)

36. The antibody of claim 3, wherein the antibody comprises a heavy chain variable (VH) region and a light chain variable (VL) region; wherein:
a) the VH region comprises an HVR-H1 comprising the amino acid sequence of SEQ ID NO:53, an HVR-H2 comprising the amino acid sequence of SEQ ID NO:54, and an HVR-H3 comprising the amino acid sequence of SEQ ID NO:55, and the VL region comprises an HVR-L1 comprising the amino acid sequence of SEQ ID NO:56, an HVR-L2 comprising the amino acid sequence of SEQ ID NO:57, and an HVR-L3 comprising the amino acid sequence of SEQ ID NO:58, or
(b) the VH region comprises an HVR-H1 comprising the amino acid sequence of SEQ ID NO:29, an HVR-H2 comprising the amino acid sequence of SEQ ID NO:30, and an HVR-H3 comprising the amino acid sequence of SEQ ID NO:31, and the VL region comprises an HVR-L1 comprising the amino acid sequence of SEQ ID NO:32, an HVR-L2 comprising the amino acid sequence of SEQ ID NO:33, and an HVR-L3 comprising the amino acid sequence of SEQ ID NO:34.

37. (canceled)

38. The antibody of claim 5, wherein the antibody comprises a heavy chain variable (VH) region and a light chain variable (VL) region; wherein:
a) the VH region comprises an HVR-H1 comprising the amino acid sequence of SEQ ID NO:23, an HVR-H2 comprising the amino acid sequence of SEQ ID NO:24, and an HVR-H3 comprising the amino acid sequence of SEQ ID NO:25, and the VL region comprises an HVR-L1 comprising the amino acid sequence of SEQ ID NO:26, an HVR-L2 comprising the amino acid sequence of SEQ ID NO:27, and an HVR-L3 comprising the amino acid sequence of SEQ ID NO:28;
(b) the VH region comprises an HVR-H1 comprising the amino acid sequence of SEQ ID NO:35, an HVR-H2 comprising the amino acid sequence of SEQ ID NO:36, and an HVR-H3 comprising the amino acid sequence of SEQ ID NO:37, and the VL region comprises an HVR-L1 comprising the amino acid sequence of SEQ ID NO:38, an HVR-L2 comprising the amino acid sequence of SEQ ID NO:39, and an HVR-L3 comprising the amino acid sequence of SEQ ID NO:40;
(c) the VH region comprises an HVR-H1 comprising the amino acid sequence of SEQ ID NO:41, an HVR-H2 comprising the amino acid sequence of SEQ ID NO:42, and an HVR-H3 comprising the amino acid sequence of SEQ ID NO:43, and the VL region comprises an HVR-L1 comprising the amino acid sequence of SEQ ID NO:44, an HVR-L2 comprising the amino acid sequence of SEQ ID NO:45, and an HVR-L3 comprising the amino acid sequence of SEQ ID NO:46;
(d) the VH region comprises an HVR-H1 comprising the amino acid sequence of SEQ ID NO:47, an HVR-H2 comprising the amino acid sequence of SEQ ID NO:48, and an HVR-H3 comprising the amino acid sequence of SEQ ID NO:49, and the VL region comprises an HVR-L1 comprising the amino acid sequence of SEQ ID NO:50, an HVR-L2 comprising the amino acid sequence of SEQ ID NO:51, and an HVR-L3 comprising the amino acid sequence of SEQ ID NO:52;
(e) the VH region comprises an HVR-H1 comprising the amino acid sequence of SEQ ID NO:59, an HVR-H2 comprising the amino acid sequence of SEQ ID NO:60, and an HVR-H3 comprising the amino acid sequence of SEQ ID NO:61, and the VL region comprises an HVR-L1 comprising the amino acid sequence of SEQ ID NO:62, an HVR-L2 comprising the amino acid sequence of SEQ ID NO:63, and an HVR-L3 comprising the amino acid sequence of SEQ ID NO:64; or
(f) the VH region comprises an HVR-H1 comprising the amino acid sequence of SEQ ID NO:65, an HVR-H2 comprising the amino acid sequence of SEQ ID NO:66, and an HVR-H3 comprising the amino acid sequence of SEQ ID NO:67, and the VL region comprises an HVR-L1 comprising the amino acid sequence of SEQ ID NO:68, an HVR-L2 comprising the amino acid sequence of SEQ ID NO:69, and an HVR-L3 comprising the amino acid sequence of SEQ ID NO:70.

39-51. (canceled)

52. The antibody of claim 1, wherein the antibody comprises a heavy chain variable (VH) region and a light chain variable (VL) region; wherein:
(a) the VH region comprises the amino acid sequence of SEQ ID NO:159, and the VL region comprises the amino acid sequence of SEQ ID NO:160;
(b) the VH region comprises the amino acid sequence of SEQ ID NO:161, and the VL region comprises the amino acid sequence of SEQ ID NO:162; or
(c) the VH region comprises the amino acid sequence of SEQ ID NO:163, and the VL region comprises the amino acid sequence of SEQ ID NO:164.

53-54. (canceled)

55. The antibody of claim 1, wherein the antibody comprises an Fc region.

56. (canceled)

57. The antibody of claim 55, wherein the Fc region is a human IgG1 or human IgG4 Fc region.

58. The antibody of claim 57, wherein:
(a) the Fc region is a human IgG4 Fc region comprising the amino acid substitution S228P, numbering according to EU index;
(b) the antibody comprises a heavy chain constant region comprising the amino acid sequence of SEQ ID NO:103; or
(c) the antibody comprises a heavy chain constant region comprising the amino acid sequence of SEQ ID NO:101 or 102.

59-60. (canceled)

61. The antibody of claim 55, wherein the Fc region comprises one or more mutation(s) that reduce effector function.

62. The antibody of claim 61, wherein the antibody comprises a human IgG1 Fc region with one or more of the following mutation(s), numbering based on EU index:
(a) L234A and/or L235A;
(b) A327G, A330S, and/or P331S;
(c) E233P, L234V, L235A, and/or G236del;
(d) E233P, L234V, and/or L235A;
(e) E233P, L234V, L235A, G236del, A327G, A330S, and/or P331S;
(f) E233P, L234V, L235A, A327G, A330S, and/or P331S;
(g) N297A;
(h) N297G;
(i) N297Q;
(j) L242C, N297C, and/or K334C;
(k) A287C, N297G, and/or L306C;
(l) R292C, N297G, and/or V302C;
(m) N297G, V323C, and/or I332C;
(n) V259C, N297G, and/or L306C;
(o) L234F, L235Q, K322Q, M252Y, S254T, and/or T256E;
(p) L234A, L235A, and/or P329G; or
(q) L234A, L235Q, and K322Q.

63. The antibody of claim 61, wherein the antibody comprises a human IgG2 Fc region with one or more of the following mutation(s), numbering based on EU index:
(a) A330S and/or P331S;
(b) V234A, G237A, P238S, H268A, V309L, A330S, and/or P331S; or
(c) V234A, G237A, H268Q, V309L, A330S, P331S, C232S, C233S, S267E, L328F, M252Y, S254T, and/or T256E.

64. The antibody of claim 61, wherein the antibody comprises a human IgG4 Fc region with one or more of the following mutation(s), numbering based on EU index:
(a) E233P, F234V, L235A, and/or G236del;
(b) E233P, F234V, and/or L235A;
(c) S228P and/or L235E; or
(d) S228P and/or L235A.

65. The antibody of claim 55, wherein the Fc region comprises one or more mutation(s) that enhance effector function.

66. The antibody of claim 65, wherein the antibody comprises a human IgG1 Fc region with one or more of the following mutation(s), numbering based on EU index:
(a) F243L, R292P, Y300L, V305I, and/or P396L;
(b) S239D and/or I332E;
(c) S239D, I332E, and/or A330L;
(d) S298A, E333A, and/or K334A;
(e) G236A, S239D, and/or I332E;
(f) K326W and/or E333S;
(g) S267E, H268F, and/or S324T; or
(h) E345R, E430G, and/or S440Y.

67. The antibody of claim 55, wherein at least one or two of the heavy chains of the antibody is non-fucosylated.

68-71. (canceled)

72. The antibody of claim 1, wherein the antibody comprises a light chain constant (CL) domain.

73. (canceled)

74. The antibody of claim 72, wherein the light chain comprises the amino acid sequence of SEQ ID NO:104.

75. The antibody of claim 1, wherein the antibody comprises a heavy chain comprising the amino acid sequence of SEQ ID NO:165 or 166 and a light chain comprising the amino acid sequence of SEQ ID NO:167.

76. (canceled)

77. The antibody of claim 75, wherein at least one or two of the heavy chains of the antibody is non-fucosylated.

78-80. (canceled)

81. The antibody of claim 1, wherein the antibody is a monoclonal antibody.

82. The antibody of claim 1, wherein the antibody is a multispecific antibody.

83. The antibody of claim 1, wherein the antibody is conjugated to an agent.

84. The antibody of claim 83, wherein the agent is a cytotoxic agent or label.

85. A composition comprising the antibody of claim 1.

86. The composition of claim 85, wherein the antibody comprises a Fc region and N-glycoside-linked carbohydrate chains linked to the Fc region, wherein less than 50% of the N-glycoside-linked carbohydrate chains contain a fucose residue.

87. The composition of claim 86, wherein substantially none of the N-glycoside-linked carbohydrate chains contain a fucose residue.

88. A polynucleotide encoding the antibody of claim 1.

89. A vector comprising the polynucleotide of claim 88.

90. A host cell comprising the polynucleotide of claim 88.

91-95. (canceled)

96. A method of producing an antibody, comprising culturing the host cell of claim 90 under a condition that produces the antibody.

97. (canceled)

98. An anti-Siglec-6 antibody produced by the method of claim 96.

99. A pharmaceutical composition comprising the antibody of claim 1 and a pharmaceutically acceptable carrier.

100. A method of treating a disease or condition characterized by increased activity and/or number of mast cells expressing Siglec-6 in a subject, inhibiting activation of mast cells expressing Siglec-6 in a subject in need thereof, or depleting mast cells expressing Siglec-6 in a subject in need thereof, comprising administering to the subject an effective amount of the composition of claim 99.

101-118. (canceled)

119. The method of claim 100, wherein the individual has or has been diagnosed with mastocytosis, mast cell leukemia, mast cell activation syndrome, gastroparesis, osteoporosis, osteopenia, renal osteodystrophy, bone fracture, Alzheimer's disease, chronic neuropathic pain, hyperalgesia, nonalcoholic fatty liver disease (NAFLD), nonalcoholic steatohepatitis (NASH), graft vs. host disease (GVH), colitis, hereditary alpha tryptasemia, neurofibroma, Kounis syndrome, urticaria, atopic dermatitis, contact dermatitis, angioedema, pruigo nodularis, cholangitis, psoriasis, irritable bowel syndrome (IBS), functional dyspepsia, asthma, allergy, keloid, chronic rhinosinusitis, aspirin exacerbated respiratory disease (AERD), chronic obstructive pulmonary disease (COPD), bullous pemphigoid, idiopathic pulmonary fibrosis, systemic sclerosis, interstitital cystitis, hidradenitis suppurativa, alopecia areata, vitiligo, mast cell gastrointestinal disease, Crohn's disease, rheumatoid arthritis, gastroesophageal reflux disease, viral infection, achalasia, postural tachycardia syndrome, amyotrophic lateral sclerosis (ALS), multiple sclerosis (MS), complex regional pain syndrome, or Ehlers-Danlos syndrome.
